(19) United States
(12) Patent Application Publication
(10) Pub. No.: US 2006/0281769 A1
(43) Pub. Date: Dec. 14, 2006

(54) SYNERGISTIC MODULATION OF FLT3 KINASE USING THIENOPYRIMIDINE AND THIENOPYRIDINE KINASE MODULATORS

(76) Inventors: Christian Andrew Baumann, Exton, PA (US); Michael David Gaul, Yardley, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/422,400

(22) Filed: Jun. 6, 2006

Related U.S. Application Data
(60) Provisional application No. 60/689,409, filed on Jun. 10, 2005.

(51) Int. Cl. A61K 48/00; A61K 31/519; A61K 31/4743

(52) U.S. Cl. 514/260.1; 514/301; 514/44

(57) ABSTRACT

The invention is directed to a method of inhibiting FLT3 tyrosine kinase activity or expression or reducing FLT3 kinase activity or expression in a cell or a subject comprising the administration of a farnesyl transferase inhibitor and a FLT3 kinase inhibitor selected from thienopyrimidine and thienopyridine compounds Formula I′ and Formula II′: 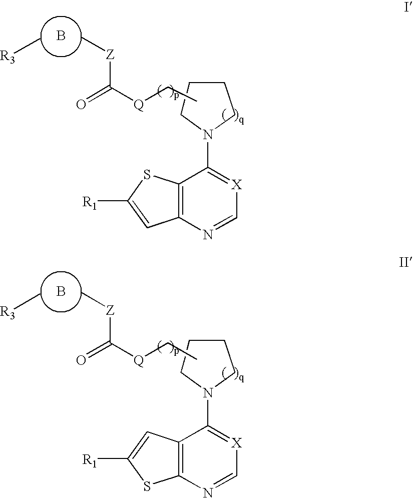
where R₁, R₃, B, Z, Q, p, q and X are as defined herein. Included within the present invention is both prophylactic and therapeutic methods for treating a subject at risk of (or susceptible to) developing a cell proliferative disorder or a disorder related to FLT3.

### CROSS REFERENCE TO RELATED APPLICATIONS

This application claims priority to U.S. Provisional Application for Patent No. 60/689,409, filed Jun. 10, 2005, the entire disclosure of which is hereby incorporated in its entirely.

### FIELD OF THE INVENTION

The present invention relates to the treatment of a cell proliferative disorder or disorders related to FLT3 using a farnesyl transferase inhibitor in combination with an inhibitor of FLT3 tyrosine kinase.

### BACKGROUND OF THE INVENTION

The fms-like tyrosine kinase 3 (FLT3) ligand (FLT3L) is one of the cytokines that affects the development of multiple hematopoietic lineages. These effects occur through the binding of FLT3L to the FLT3 receptor, also referred to as fetal liver kinase-2 (flk-2) and STK-1, a receptor tyrosine kinase (RTK) expressed on hematopoietic stem and progenitor cells. The FLT3 gene encodes a membrane-spanning class III RTK that plays an important role in proliferation, differentiation and apoptosis of cells during normal hematopoiesis. The FLT3 gene is mainly expressed by early myeloid and lymphoid progenitor cells. See McKenna, Hilary J. et al. Mice lacking flt3 ligand have deficient hematopoiesis affecting hematopoietic progenitor cells, dendritic cells, and natural killer cells. Blood. June 2000; 95: 3489-3497; Drexler, H. G. and H. Quentmeier (2004). “FLT3: receptor and ligand.” Growth Factors 22(2): 71-3.

The ligand for FLT3 is expressed by the marrow stromal cells and other cells and synergizes with other growth factors to stimulate proliferation of stem cells, progenitor cells, dendritic cells, and natural killer cells.

Hematopoietic disorders are pre-malignant disorders of these systems and include, for instance, the myeloproliferative disorders, such as thrombocythemia, essential thrombocytosis (ET), angiogenic myeloid metaplasia, myelofibrosis (MF), myelofibrosis with myeloid metaplasia (MMM), chronic idiopathic myelofibrosis (IMF), polycythemia vera (PV), the cytopenias, and pre-malignant myelodysplastic syndromes. See Stirewalt, D. L. and J. P. Radich (2003). “The role of FLT3 in haematopoietic malignancies.” Nat Rev Cancer 3(9): 650-65; Scheijen, B. and J. D. Griffin (2002). “Tyrosine kinase oncogenes in normal hematopoiesis and hematological disease.” Oncogene 21(21): 3314-33.

Hematological malignancies are cancers of the body's blood forming and immune systems, the bone marrow and lymphatic tissues. Whereas in normal bone marrow, FLT3 expression is restricted to early progenitor cells, in hematological malignancies, FLT3 is expressed at high levels or FLT3 mutations cause an uncontrolled induction of the FLT3 receptor and downstream molecular pathway, possibly Ras activation. Hematological malignancies include leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), multiple myeloma, (MM) and myeloid sarcoma. See Kottaridis, P. D., R. E. Gale, et al. (2003). “Flt3 mutations and leukaemia.” Br J Haematol 122(4): 523-38. Myeloid sarcoma is also associated with FLT3 mutations. See Ansari-Lari, Ali et al. FLT3 mutations in myeloid sarcoma. British Journal of Haematology. 2004 September 126(6):785-91.

Acute Myelogenous Leukemia (AML) is the most prevalent form of adult leukemia and represents 15-20% of childhood leukemias. In 2002, in the United States, approximately 11,000 new cases of AML were diagnosed and an estimated 8,000 patients died from AML. See National Cancer Institute SEER database—http://seer.cancer.gov/. Although diagnosis for AML is traditionally based on histological techniques and blood leukocyte count, recent advances in cytogenetic and genetic analysis have revealed that AML is a mixture of distinct diseases that differ in their genetic abnormalities, clinical features and response to therapy. Recent efforts have begun to tailor chemotherapy to the different sub-types of AML (subtypes are based on cytogenetic analysis and immunohistochemical analysis for disease associated protein expression) with some success. Treatment of AML typically occurs in two phases: induction and post-induction therapy. Induction therapy typically consists of three doses of an anthracycline such as daunorubicin followed by i.v. bolus infusion of the cytotoxic cytarabine for 7-10 days. This regime is effective at inducing remission in 70-80% of patient <60 years of age and ˜50% of patients >60. See Burnett, A. K. (2002). “Acute myeloid leukemia: treatment of adults under 60 years.” Rev Clin Exp Hematol 6(1): 26-45; Buchner T., W. Hiddemann, et al. (2002). “Acute myeloid leukemia: treatment over 60.” Rev Clin Exp Hematol. 6(1):46-59. After remission induction there are several post-induction options including an additional cycle of chemotherapy or bone marrow transplantation. Post-induction treatment choice and success depends on the patient's age and AML sub-type. Despite the advances in diagnosis and treatment of AML over the last decade, the 5 year disease free survival for patients under 65 is only 40% and the 5 year disease free survival of patients over 65 is less than 10% percent. Thus, there remains a significant unmet clinical need for AML particularly in patients over 65. With the increased knowledge of the mechanisms of the different sub-types of AML new tailored treatments for the disease are beginning to immerge with some promising results.

One recent success in relapse and refractory AML treatment is the development and use of farnesyl transferase inhibitors (FTI) for post-induction treatment. Farnesyl transferase inhibitors are a potent and selective class of inhibitors of intracellular farnesyl protein transferase (FPT). FPT catalyses the lipid modification of a host of intracellular proteins, including the small GTPases of the Ras and Rho family and lamin proteins, to direct their localization to the plasma membrane or membrane compartments within the cell.

FTIs were originally developed to prevent post-translational farnesylation and activation of Ras oncoproteins (Prendergast G. C. and Rane, N. (2001) “Farnesyl Transferase Inhibtors: Mechanism and Applications” Expert Opin Investig Drugs. 10(12):2105-16). Recent studies also demonstrate FTI induced inhibition of Nf-κB activation leading to increased sensitivity to induction of apoptosis and downregulation of inflammatory gene expression through suppression of Ras-dependent Nf-κB activation. See Takada, Y., et al. (2004). “Protein farnesyltransferase inhibitor (SCH 66336) abolishes NF-kappaB activation induced by various carcinogens and inflammatory stimuli leading to suppression of NF-kappaB-regulated gene expression and up-regulation of apoptosis.” J Biol Chem 279, 26287-99.

Of particular interest for oncology, FTI inhibition of the oncogenes of the Ras and Rho family leads to growth arrest and apoptosis of tumor cells both in vitro and in vivo. See Haluska P., G. K. Dy, A. A. Adjei. (2002) “Farnesyl transferase inhibitors as anticancer agents.” Eur J Cancer. 38(13):1685-700. From a clinical perspective, myeloid malignancies, particularly AML, represent a significant opportunity for FTI therapy.

As discussed earlier, AML is a disease with very low long-term survival and an elevated rate of chemotherapy-induced toxicity and resistance (particularly in patients >60 years of age). Additionally, the mechanism of proliferation of AML cells relies on the small GTPases of the Ras and Rho family. With the plethora of pre-clinical data supporting the efficacy of FTIs in AML treatment, several clinical trials were initiated with an FTI including; Tipifarnib (Zarnestra™, Johnson and Johnson), BMS-214662, CP-60974 (Pfizer) and Sch-6636 (lonafarnib, Schering-Plough).

ZARNESTRA® (also known as R115777 or Tipifarnib) is the most advanced and promising of the FTI class of compounds. In clinical studies of patients with relapsed and refractory AML, Tipifarnib treatment resulted in a ˜30% response rate with 2 patients achieving complete remission. See Lancet J. E., J. D. Rosenblatt, J. E. Karp. (2003) “Farnesyltransferase inhibitors and myeloid malignancies: phase I evidence of Zarnestra activity in high-risk leukemias.” Semin Hematol. 39(3 Suppl 2):31-5. These responses occurred independently of the patients Ras mutational status, as none of the patients in the trial had the Ras mutations that are sometimes seen in AML patients. However, there was a direct correlation of patient responses to their level of MAPkinase activation (a downstream target of both Ras and Rho protein activity) at the onset of treatment, suggesting that the activity of the Ras/MAPkinase pathway, activated by other mechanisms may be a good predictor of patient responses. See Lancet J. E., J. D. Rosenblatt, J. E. Karp. (2003) “Farnesyltransferase inhibitors and myeloid malignancies: phase I evidence of Zarnestra activity in high-risk leukemias.” Semin Hematol. 39(3 Suppl 2): 31-5. Additionally, a recent multicenter Phase II trial in patients with relapsed AML demonstrated complete responses (bone marrow blasts <5%) in 17 of 50 patients and a>50% reduction in bone marrow blasts in 31 of 50 patients. Reviewed in Gotlib, J (2005) “Farnesyltransferase inhibitor therapy in acute myelogenous leukemia.” Curr. Hematol. Rep.; 4(1):77-84. Preliminary analysis of genes regulated by the FTI treatment in responders in that trial also demonstrated an effect on proteins in the MAPKinase pathway. This promising result has experts in the field anticipating the use of Tipifarnib in the clinic in the near future.

Recently, another target for the treatment of AML, and a subset of patients with MDS and ALL, has emerged. The receptor tyrosine kinase, FLT3 and mutations of FLT3, have been identified as key player in the progression of AML. A summary of the many studies linking FLT3 activity to disease have been extensively reviewed by Gilliland, D. G. and J. D. Griffin (2002). “The roles of FLT3 in hematopoiesis and leukemia.” Blood 100(5): 1532-42, and Stirewalt, D. L. and J. P. Radich (2003). “The role of FLT3 in haematopoietic malignancies.” Nat Rev Cancer 3(9): 650-65. Greater than 90% of patients with AML have FLT3 expression in blast cells. It is now known that roughly 30-40% of patients with AML have an activating mutation of FLT3, making FLT3 mutations the most common mutation in patients with AML. There are two known types of activating mutations of FLT3. One is a duplication of 4-40 amino acids in the juxtamembrane region (ITD mutation) of the receptor (25-30% of patients) and the other is a point mutation in the kinase domain (5-7% of patients). These receptor mutations cause constituitive activation of multiple signal transduction pathways including Ras/MAPkinase, PI3kinase/AKT, and the STAT pathways. Additionally, the FLT31TD mutation also has been shown to decrease the differentiation of early myeloid cells. More significantly, patients with the ITD mutation have decreased rates of remission induction, decreased remission times, and poorer overall prognosis. FLT31TD mutations have also been found in ALL with the MLL gene rearrangement and in a sub-population of MDS patients. The presence of the FLT31TD mutation in MDS and ALL is also correlated with accelerated disease progression and poorer prognosis in these patients. See Shih L. Y. et al., (2004) “Internal tandem duplication of fins-like tyrosine kinase 3 is associated with poor outcome in patients with myelodysplastic syndrome.” Cancer, 101; 989-98; and Armstrong, S. A. et al., (2004) “FLT3 mutations in childhood acute lymphoblastic leukemia.” Blood. 103: 3544-6. To date, there is no strong evidence that suggests either the kinase domain point mutations or the over expressed wild-type receptor is causative of disease, however, FLT3 expression may contribute to the progression of the disease. This building pre-clinical and clinical evidence has led to the development of a number of FLT3 inhibitors which are currently being evaluated in the pre-clinical and clinical setting.

An emerging strategy for the treatment of AML is the combination of target directed therapeutic agents together or with conventional cytotoxic agents during induction and/or post-induction therapy. Recent proof of concept data has been published that demonstrate the combination of the cytotoxic agents (such as cytarabine or daunorubicin) and FLT3 inhibitors inhibit the growth of AML cells expressing FLT31TD. See Levis, M., R. Pham, et al. (2004). “In vitro studies of a FLT3 inhibitor combined with chemotherapy: sequence of administration is important to achieve synergistic cytotoxic effects.” Blood 104(4): 1145-50, and Yee K W, Schittenhelm M, O'Farrell A M, Town A R, McGreevey L, Bainbridge T, Cherrington J M, Heinrich M C. (2004) “Synergistic effect of SU11248 with cytarabine or daunorubicin on FLT31TD-positive leukemic cells.” Blood. 104(13):4202-9.

Accordingly, the present invention provides a synergistic method of treatment comprising co-administration (simultaneous or sequential) of a novel FLT3 kinase inhibitor described herein and a farnesyl transferase inhibitor for the treatment of FLT3 expressing cell proliferative disorders.

A variety of FTase inhibitors are currently known. FTIs appropriate for use in the present invention are the following: WO-97/21701 and U.S. Pat. No. 6,037,350, which are incorporated herein in their entirety, describe the preparation, formulation and pharmaceutical properties of certain farnesyl transferase inhibiting (imidazoly-5-yl)methyl-2-quinolinone derivatives of formulas (I), (II) and (III), as well as intermediates of formula (II) and (III) that are metabolized in vivo to the compounds of formula (I). The compounds of formulas (I), (II) and (III) are represented by 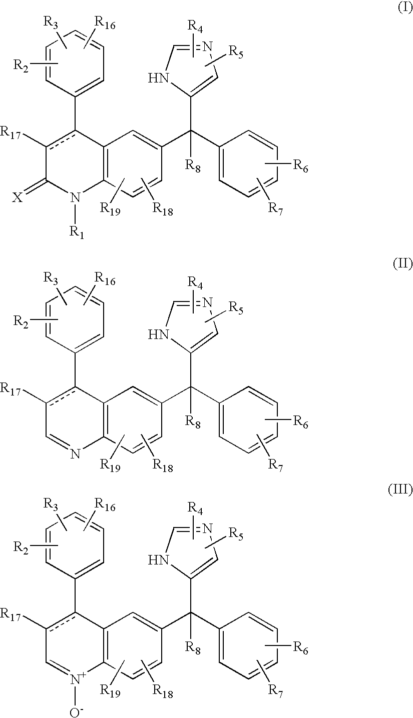
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
   wherein
      Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula
   —O—CH₂—O— (a-1),
   —O—CH₂—CH₂—O— (a-2),
   —O—CH═CH— (a-3),
   —O—CH₂—CH₂— (a-4),
   —O—CH₂—CH₂—CH₂— (a-5), or
   —CH═CH—CH═CH— (a-6);
R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or
when on adjacent positions R⁶ and R⁷ taken together may form a bivalent radical of formula
   —O—CH₂—O— (c-1), or
   —CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula
   —O—R¹⁰ (b-1),
   —S—R¹⁰ (b-2),
   —N—R¹¹R¹² (b-3),
   wherein
      R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical of formula -Alk²-OR¹³ or -Alk²-NR¹⁴R¹⁵;
      R¹¹ is hydrogen, C₁₋₁₂alkyl, Ar¹ or Ar²C₁₋₆alkyl;
      R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical of formula -Alk²-OR¹³ or -Alk²-NR¹⁴R¹⁵;
   wherein
      Alk² is C₁₋₆alkanediyl;
         R¹³ is hydrogen, C₁₋₆alkyl, C₁₋₆alkylcarbonyl, hydroxyC₁₋₆alkyl, Ar¹ or Ar²C₁₋₆alkyl;
         R¹⁴ is hydrogen, C₁₋₆alkyl, Ar¹ or Ar²C₁₋₆alkyl;
         R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

WO-97/16443 and U.S. Pat. No. 5,968,952, which are incorporated herein in their entirety, describe the preparation, formulation and pharmaceutical properties of farnesyltransferase inhibiting compounds of formula (IV), as well as intermediates of formula (V) and (VI) that are metabolized in vivo to the compounds of formula (IV). The compounds of formulas (IV), (V) and (VI) are represented by 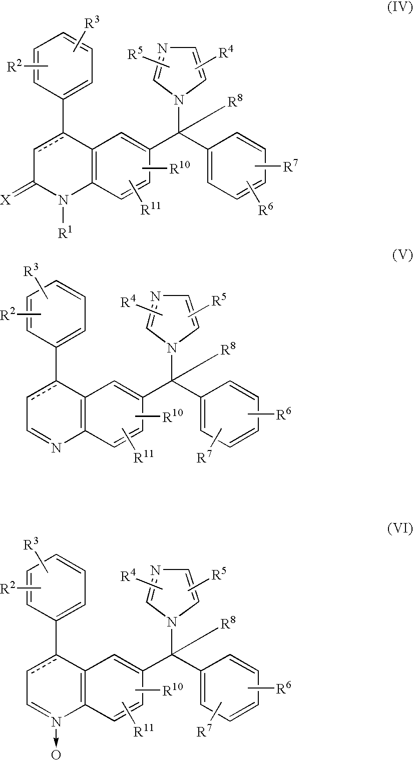
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
   wherein
      Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or
when on adjacent positions R² and R³ taken together may form a bivalent radical of formula
   —O—CH₂—O— (a-1),
   —O—CH₂—CH₂—O— (a-2),
   —O—CH═CH— (a-3),
   —O—CH₂—CH₂— (a-4),
   —O—CH₂—CH₂—CH₂— (a-5), or
   —CH═CH—CH═CH— (a-6);
R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy or Ar²oxy;
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, hydroxycarbonylC₁₋₆alkyl, hydroxyC₁₋₆alkyl, aminoC₁₋₆alkyl, mono- or di(C₁₋₆alkyl)aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;
R¹⁰ is hydrogen, C₁₋₆alkyl, C₁₋₆alkyloxy or halo;
R¹¹ is hydrogen or C₁₋₆alkyl;
Ar¹ is phenyl or phenyl substituted with C₁₋₆alkyl, hydroxy, amino, C₁₋₆alkyloxy or halo;
Ar² is phenyl or phenyl substituted with C₁₋₆alkyl, hydroxy, amino, C₁₋₆alkyloxy or halo.

WO-98/40383 and U.S. Pat. No. 6,187,786, which are incorporated herein in their entirety, disclose the preparation, formulation and pharmaceutical properties of farnesyltransferase inhibiting compounds of formula (VII) 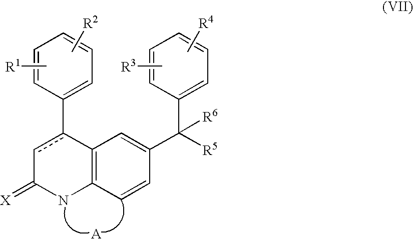
the pharmaceutically acceptable acid addition salts and the stereochemically isomeric forms thereof, wherein
the dotted line represents an optional bond;
X is oxygen or sulfur;
-A- is a bivalent radical of formula
   —CH═CH— (a-1),
   —CH₂—CH₂— (a-2),
   —CH₂—CH₂—CH₂— (a-3),
   —CH₂—O— (a-4),
   —CH₂—CH₂—O— (a-5),
   —CH₂—S— (a-6),
   —CH₂—CH₂—S— (a-7),
   —CH═N— (a-8),
   —N═N— (a-9),
   or
   —CO—NH— (a-10);
wherein optionally one hydrogen atom may be replaced by C₁₋₄alkyl or Ar¹;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²—C₁₋₆alkyl, Ar²-oxy, Ar²—C₁₋₆alkyloxy; or when on adjacent positions R¹ and R² taken together may form a bivalent radical of formula
   —O—CH₂—O— (b-1),
   —O—CH₂—CH₂—O— (b-2),
   —O—CH═CH— (b-3),
   —O—CH₂—CH₂— (b-4),
   —O—CH₂—CH₂—CH₂— (b-5), or
   —CH═CH—CH═CH— (b-6);
R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula
   —O—CH₂—O— (c-1),
   —O—CH₂—CH₂—O— (c-2), or
   —CH═CH—CH═CH— (c-3);
R⁵ is a radical of formula 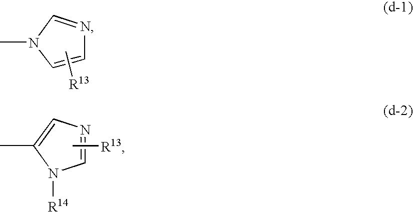
   wherein
      R¹³ is hydrogen, halo, Ar⁴, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylS(O)C₁₋₆alkyl or C₁₋₆alkylS(O)₂C₁₋₆alkyl;
      R¹⁴ is hydrogen, C₁₋₆alkyl or di(C₁₋₄alkyl)aminosulfonyl;
R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵—C₁₋₆alkyloxyC₁₋₆alkyl; or a radical of formula
   —O—R⁷ (e-1),
   —S—R⁷ (e-2),
   —N—R⁸R⁹ (e-3),
   wherein
      R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;
      R⁸ is hydrogen, C₁₋₆alkyl, Ar⁷ or Ar⁷—C₁₋₆alkyl;
      R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical of formula -Alk-OR¹⁰ or -Alk-NR¹¹R¹²;
   wherein
      Alk is C₁₋₆alkanediyl;
      R¹⁰ is hydrogen, C₁₋₆alkyl, C₁₋₆alkylcarbonyl, hydroxyC₁₋₆alkyl, Ar⁹ or Ar⁹—C₁₋₆alkyl;
      R¹¹ is hydrogen, C₁₋₆alkyl, C₁₋₆alkylcarbonyl, Ar¹⁰ or Ar¹⁰—C₁₋₆alkyl;
      R¹² is hydrogen, C₁₋₆alkyl, Ar¹¹ or Ar¹¹—C₁₋₆alkyl; and
   Ar¹ to Ar¹¹ are each independently selected from phenyl; or phenyl substituted with halo, C₁₋₆alkyl, C₁₋₆alkyloxy or trifluoromethyl.

WO-98/49157 and U.S. Pat. No. 6,117,432, which are incorporated herein in their entirety, concern the preparation, formulation and pharmaceutical properties of farnesyltransferase inhibiting compounds of formula (VIII) 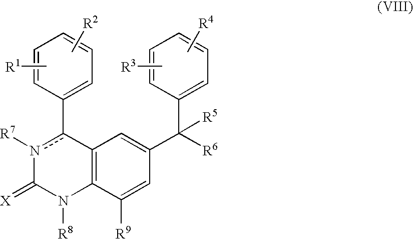
the pharmaceutically acceptable acid addition salts and the stereochemically isomeric forms thereof, wherein
the dotted line represents an optional bond;
X is oxygen or sulfur;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;
R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;
R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula
   —O—R¹⁰ (a-1),
   —S—R¹⁰ (a-2),
   —N—R¹¹R¹² (a-3),
   wherein
      R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;
      R¹¹ is hydrogen, C₁₋₆alkyl, Ar¹ or Ar¹C₁₋₆alkyl;
      R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical of formula -Alk-OR¹³ or -Alk-NR¹⁴R¹⁵;
         wherein
            Alk is C₁₋₆alkanediyl;
            R¹³ is hydrogen, C₁₋₆alkyl, C₁₋₆alkylcarbonyl, hydroxyC₁₋₆alkyl, Ar¹ or Ar¹ C₁₋₆alkyl;
            R¹⁴ is hydrogen, C₁₋₆alkyl, Ar¹ or Ar¹C₁₋₆alkyl;
            R¹⁵ is hydrogen, C₁₋₆alkyl, C₁₋₆alkylcarbonyl, Ar¹ or Ar¹C₁₋₆alkyl;
R⁶ is a radical of formula 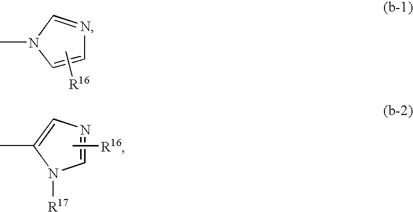
   wherein
      R¹⁶ is hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
      R¹⁷ is hydrogen, C₁₋₆alkyl or di(C₁₋₄alkyl)aminosulfonyl;
R⁷ is hydrogen or C₁₋₆alkyl provided that the dotted line does not represent a bond;
R⁸ is hydrogen, C₁₋₆alkyl or Ar²CH₂ or Het¹CH₂;
R⁹ is hydrogen, C₁₋₆alkyl, C₁₋₆alkyloxy or halo; or
R⁸ and R⁹ taken together to form a bivalent radical of formula
   —CH═CH— (c-1),
   —CH₂—CH₂— (c-2),
   —CH₂—CH₂—CH₂— (c-3),
   —CH₂—O— (c-4), or
   —CH₂—CH₂—O— (c-5);
Ar¹ is phenyl; or phenyl substituted with 1 or 2 substituents each independently selected from halo, C₁₋₆alkyl, C₁₋₆alkyloxy or trifluoromethyl;
Ar² is phenyl; or phenyl substituted with 1 or 2 substituents each independently selected from halo, C₁₋₆alkyl, C₁₋₆alkyloxy or trifluoromethyl; and
Het¹ is pyridinyl; pyridinyl substituted with 1 or 2 substituents each independently selected from halo, C₁₋₆alkyl, C₁₋₆alkyloxy or trifluoromethyl.

WO-00/39082 and U.S. Pat. No. 6,458,800, which are incorporated herein in their entirety, describe the preparation, formulation and pharmaceutical properties of farnesyltransferase inhibiting compounds of formula (IX) 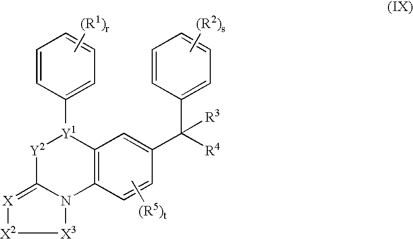
or the pharmaceutically acceptable acid addition salts and the stereochemically isomeric forms thereof, wherein
═X¹—X²—X³— is a trivalent radical of formula
   ═N—CR⁶═CR⁷— (x-1),
   ═N—N═CR⁶— (x-2),
   ═N—NH—C(═O)— (x-3),
   ═N—N═N— (x-4),
   ═N—CR⁶═N— (x-5),
   ═CR⁶—CR⁷═CR⁸— (x-6),
   ═CR⁶—N═CR⁷— (x-7),
   ═CR⁶—NH—C(═O)— (x-8),
   or
   ═CR⁶—N═N— (x-9);
wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;
>Y¹—Y²— is a trivalent radical of formula
   >CH—CHR⁹— (y-1),
   >C═N— (y-2),
   >CH—NR⁹— (y-3), or
   >C═CR⁹— (y-4);
wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;
r and s are each independently 0, 1, 2, 3, 4 or 5;
t is 0, 1, 2 or 3;
each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or
two R¹ or R² substituents adjacent to one another on the phenyl ring may independently form together a bivalent radical of formula
   —O—CH₂—O— (a-1),
   —O—CH₂—CH₂—O— (a-2),
   —O═CH═CH— (a-3),
   —O—CH₂—CH₂— (a-4),
   —O—CH₂—CH₂—CH₂— (a-5), or
   —CH═CH—CH═CH— (a-6);
R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;
or a radical of formula
   —O—R¹⁰ (b-1), —S—R₁₀ (b-2),
   —NR¹¹R¹² (b-3),
wherein
   R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;
   R¹¹ is hydrogen, C₁₋₆alkyl, aryl or arylC₁₋₆alkyl;
   R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl,
   C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or
   di(C₁₋₆alkyl)aminoC₁₋₆alkylcarbonyl, or a radical of formula -Alk-OR¹³ or -Alk-NR¹⁴R¹⁵;
   wherein
      Alk is C₁₋₆alkanediyl;
      R¹³ is hydrogen, C₁₋₆alkyl, C₁₋₆alkylcarbonyl, hydroxyC₁₋₆alkyl, aryl or arylC₁₋₆alkyl;
      R¹⁴ is hydrogen, C₁₋₆alkyl, aryl or arylC₁₋₆alkyl;
      R¹⁵ is hydrogen, C₁₋₆alkyl, C₁₋₆alkylcarbonyl, aryl or arylC₁₋₆alkyl;
R⁴ is a radical of formula 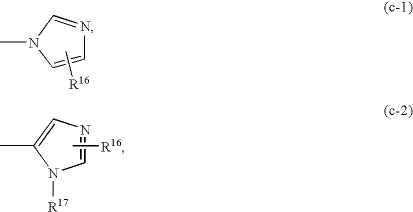
wherein
   R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
   R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;
   R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;
R⁵ is C₁₋₆alkyl, C₁₋₆alkyloxy or halo;
aryl is phenyl, naphthalenyl or phenyl substituted with 1 or more substituents each independently selected from halo, C₁₋₆alkyl, C₁₋₆alkyloxy or trifluoromethyl

In addition to the farnesyltransferase inhibitors of formula (I), (II), (III), (IV), (V), (VI), (VII), (VIII) or (IX) above, other farnesyltransferase inhibitors known in the art include: Arglabin (i.e. 1(R)-10-epoxy-5(S),7(S)-guaia-3(4),11(13)-dien-6,12-olide described in WO-98/28303 (NuOncology Labs); perrilyl alcohol described in WO-99/45912 (Wisconsin Genetics); SCH-66336, i.e. (+)-(R)-4-[2-[4-(3,10-dibromo-8-chloro-5,6-dihydro-11H-benzo[5,6]cyclohepta[1,2-b]pyridin-11-yl)piperidin-1-yl]-2-oxoethyl]piperidine-1-carboxamide, described in U.S. Pat. No. 5,874,442 (Schering); L778123, i.e. 1-(3-chlorophenyl)-4-[1-(4-cyanobenzyl)-5-imidazolylmethyl]-2-piperazinone, described in WO-00/01691 (Merck); compound 2(S)-[2(S)-[2(R)-amino-3-mercapto]propylamino-3(S)-methyl]-pentyloxy-3-phenylpropionyl-methionine sulfone described in WO-94/10138 (Merck); and BMS 214662, i.e. (R)-2,3,4,5-tetrahydro-1-(1H-imidazol-4-ylmethyl)-3-(phenylmethyl)-4-(2-thienylsulphonyl)-1H-1,4-benzodiazapine-7-carbonitrile, described in WO 97/30992 (Bristol Myers Squibb); and Pfizer compounds (A) and (B) described in WO-00/12498 and WO-00/12499:

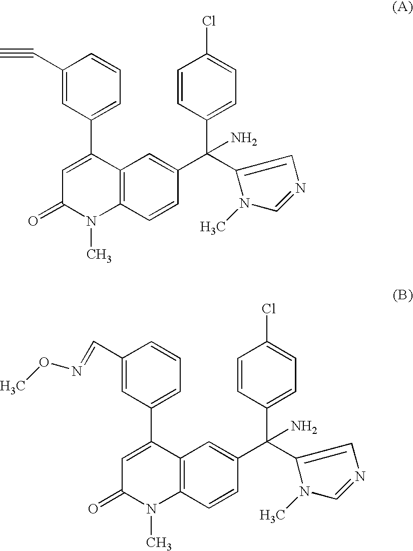

FLT3 kinase inhibitors known in the art include: AG1295 and AG1296; Lestaurtinib (also known as CEP 701, formerly KT-5555, Kyowa Hakko, licensed to Cephalon); CEP-5214 and CEP-7055 (Cephalon); CHIR-258 (Chiron Corp.); EB-10 and IMC-EB10 (ImClone Systems Inc.); GTP 14564 (Merk Biosciences UK). Midostaurin (also known as PKC 412 Novartis AG); MLN 608 (Millennium USA); MLN-518 (formerly CT53518, COR Therapeutics Inc., licensed to Millennium Pharmaceuticals Inc.); MLN-608 (Millennium Pharmaceuticals Inc.); SU-11248 (Pfizer USA); SU-11657 (Pfizer USA); SU-5416 and SU 5614; THRX-165724 (Theravance Inc.); AMI-10706 (Theravance Inc.); VX-528 and VX-680 (Vertex Pharmaceuticals USA, licensed to Novartis (Switzerland), Merck & Co USA); and XL 999 (Exelixis USA).

See also Levis, M., K. F. Tse, et al. (2001) “A FLT3 tyrosine kinase inhibitor is selectively cytotoxic to acute myeloid leukemia blasts harboring FLT3 internal tandem duplication mutations.” Blood 98(3): 885-7; Tse K F, et al. (2001) Inhibition of FLT3-mediated transformation by use of a tyrosine kinase inhibitor. Leukemia. July; 15(7):1001-10; Smith, B. Douglas et al. Single-agent CEP-701, a novel FLT3 inhibitor, shows biologic and clinical activity in patients with relapsed or refractory acute myeloid leukemia Blood, May 2004; 103: 3669-3676; Griswold, Ian J. et al. Effects of MLN518, A Dual FLT3 and KIT Inhibitor, on Normal and Malignant Hematopoiesis. Blood, July 2004; [Epub ahead of print]; Yee, Kevin W. H. et al. SU5416 and SU5614 inhibit kinase activity of wild-type and mutant FLT3 receptor tyrosine kinase. Blood, September 2002; 100: 2941-294; O'Farrell, Anne-Marie et al. SU11248 is a novel FLT3 tyrosine kinase inhibitor with potent activity in vitro and in vivo. Blood, May 2003; 101: 3597-3605; Stone, R. M. et al. PKC 412 FLT3 inhibitor therapy in AML: results of a phase II trial. Ann Hematol. 2004; 83 Suppl 1:S89-90; and Murata, K. et al. Selective cytotoxic mechanism of GTP-14564, a novel tyrosine kinase inhibitor in leukemia cells expressing a constitutively active Fms-like tyrosine kinase 3 (FLT3). J Biol. Chem. 2003 Aug. 29; 278(35):32892-8; Levis, Mark et al. Novel FLT3 tyrosine kinase inhibitors. Expert Opin. Investing. Drugs (2003) 12(12) 1951-1962; Levis, Mark et al. Small Molecule FLT3 Tyrosine Kinase Inhibitors. Current Pharmaceutical Design, 2004, 10, 1183-1193.

### SUMMARY OF THE INVENTION

The present invention comprises a method of inhibiting FLT3 tyrosine kinase activity or expression or reducing FLT3 kinase activity or expression in a cell or a subject comprising the administration of a FLT3 kinase inhibitor and a farnesyl transferase inhibitor. Included within the present invention is both prophylactic and therapeutic methods for treating a subject at risk of (or susceptible to) developing a cell proliferative disorder or a disorder related to FLT3, the methods comprising generally administering to the subject a prophylactically effective amount of a FLT3 kinase inhibitor and a farnesyl transferase inhibitor. The FLT3 kinase inhibitor and farnesyl transferase inhibitor can be administered as a unitary pharmaceutical composition comprising a FLT3 kinase inhibitor, a farnesyl transferase inhibitor and a pharmaceutically acceptable carrier, or as separate pharmaceutical compositions: (1) a first pharmaceutical composition comprising a FLT3 kinase inhibitor and a pharmaceutically acceptable carrier, and (2) a second pharmaceutical composition comprising a farnesyl transferase inhibitor and a pharmaceutically acceptable carrier.

The invention further encompasses a multiple component therapy for treating or inhibiting onset of a cell proliferative disorder or a disorder related to FLT3 in a subject comprising administering to the subject a therapeutically or prophylactically effective amount of a FLT3 kinase inhibitor, a farnesyl transferase inhibitor and one or more other anti-cell proliferation therapy(ies) including chemotherapy, radiation therapy, gene therapy and immunotherapy.

Other embodiments, features, advantages, and aspects of the invention will become apparent from the detailed description hereafter in reference to the drawing figures.

### DESCRIPTION OF THE DRAWINGS

FIG. 1. Effects of oral administration of compounds of the present invention on the growth of MV4-11 tumor xenografts in nude mice.

FIG. 2. Effects of oral administration of compounds of the present invention on the final weight of MV4-11 tumor xenografts in nude mice.

FIG. 3. FLT3 phosphorylation in MV4-11 tumors obtained from mice treated with compounds of the present invention.

FIG. 4. FIG. 4 is intentionally omitted.

FIG. 5. Compounds tested for inhibition of FLT3-dependent proliferation.

FIG. 6.1-6.8. Dose responses of single agents on FLT3 dependent AML cell proliferation.

FIG. 7a-c. A low dose of a FLT3 inhibitor significantly shifts the potency of Tipifarnib in FLT3 dependent cells.

FIG. 8a-d. Single dose combinations of a FLT3 inhibitor Compound (A) and Tipifarnib or Cytarabine synergistically inhibit FLT3-dependent cell line growth.

FIG. 9a-b. Single dose combination of FLT3 inhibitor Compounds B and D with either Tipifarnib or Cytarabine synergistically inhibits MV4-11 cell growth.

FIG. 10.1. FLT3 inhibitor Compound A and Tipifarnib synergistically inhibit the proliferation of FLT3 dependent cells as measured by the method of Chou ad Talalay.

FIG. 10.2. FLT3 inhibitor Compound B and Tipifarnib synergistically inhibit the proliferation of FLT3 dependent cells as measured by the method of Chou ad Talalay.

FIG. 10.3. FLT3 inhibitor Compound C and Tipifarnib synergistically inhibit the proliferation of FLT3 dependent cells as measured by the method of Chou ad Talalay.

FIG. 10.4. FLT3 inhibitor Compound D and Tipifarnib synergistically inhibit the proliferation of FLT3 dependent cells as measured by the method of Chou ad Talalay.

FIG. 10.5. FLT3 inhibitor Compound H and Tipifarnib synergistically inhibit the proliferation of MV4-11 cells as measured by the method of Chou and Talalay.

FIG. 10.6. FLT3 inhibitor Compound E and Zarnestra synergistically inhibit the proliferation of MV4-11 cells as measured by the method of Chou and Talalay.

FIG. 10.7. FLT3 inhibitor Compound F and Tipifarnib synergistically inhibit the proliferation of FLT3 dependent MV4-11 cells as measured by the method of Chou ad Talalay.

FIG. 10.8. FLT3 inhibitor Compound G and Tipifarnib synergistically inhibit the proliferation of FLT3 dependent MV4-11 cells as measured by the method of Chou ad Talalay.

FIG. 11a-c. The combination of a FLT3 inhibitor and an FTI synergistically induces apoptosis of MV4-11 cells.

FIG. 12 a-d. Dose responses of single agent induction of caspase 3/7 activation and apoptosis of FLT3 dependent MV4-11 cells.

FIG. 13.1. FLT3 inhibitor Compound B and Tipifamib synergistically induce the activation of caspase 3/7 in FLT3 dependent MV4-11 cells as measured by the method of Chou ad Talalay.

FIG. 13.2. FLT3 inhibitor Compound C and Tipifamib synergistically induce the activation of caspase 3/7 in FLT3 dependent MV4-11 cells as measured by the method of Chou ad Talalay.

FIG. 13.3. FLT3 inhibitor Compound D and Tipifamib synergistically induce the activation of caspase 3/7 in FLT3 dependent MV4-11 cells as measured by the method of Chou ad Talalay.

FIG. 14. Tipifamib increases the potency of FLT3 inhibitor Compound A inhibition of FLT3 and MapKinase phosphorylation in MV4-11 cells.

FIG. 15. Effects over time on tumor volume of orally administered FLT3 inhibitor Compound B and Tipifarnib, alone and in combination, on the growth of MV-4-11 tumor xenografts in nude mice.

FIG. 16. Effects on tumor volume of orally administered FLT3 inhibitor Compound B and Tipifarnib alone or in combination on the growth of MV-4-11 tumor xenografts in nude mice at the terminal study day.

FIG. 17. Effects on tumor weight of orally administered FLT3 inhibitor Compound B and Tipifarnib alone or in combination on the growth of MV-4-11 tumor xenografts in nude mice at the terminal study day.

FIG. 18. Effects of oral administration of FLT3 inhibitor Compound D of the present invention on the growth of MV4-11 tumor xenografts in nude mice.

FIG. 19. Effects of oral administration of FLT3 inhibitor Compound D of the present invention on the final weight of MV4-11 tumor xenografts in nude mice.

FIG. 20. Effects of oral administration of FLT3 inhibitor Compound D of the present invention on mouse body weight.

FIG. 21. FLT3 phosphorylation in MV4-11 tumors obtained from mice treated with FLT3 inhibitor Compound D of the present invention.

FIG. 22. Effects over time on tumor volume of orally administered FLT3 inhibitor Compound D and Tipifarnib, alone and in combination, on the growth of MV-4-11 tumor xenografts in nude mice.

FIG. 23. Effects on tumor volume of orally administered FLT3 inhibitor Compound D and Tipifarnib alone or in combination on the growth of MV-4-11 tumor xenografts in nude mice.

FIG. 24. Effects of orally administered FLT3 inhibitor Compound D and Tipifarnib alone or in combination on the final weight of MV-4-11 tumor xenografts in nude mice.

### DETAILED DESCRIPTION OF THE INVENTION AND PREFERRED EMBODIMENTS

The terms “comprising”, “including”, and “containing” are used herein in their open, non-limited sense.

The present invention comprises a method of inhibiting FLT3 tyrosine kinase activity or expression or reducing FLT3 kinase activity or expression in a cell or a subject comprising the administration of a FLT3 kinase inhibitor and a farnesyl transferase inhibitor.

An embodiment of the present invention comprises a method for reducing or inhibiting FLT3 tyrosine kinase activity in a subject comprising the administration of a FLT3 kinase inhibitor and a farnesyl transferase inhibitor to the subject.

An embodiment of the present invention comprises a method of treating disorders related to FLT3 tyrosine kinase activity or expression in a subject comprising the administration of a FLT3 kinase inhibitor and a farnesyl transferase inhibitor to the subject.

An embodiment of the present invention comprises a method for reducing or inhibiting the activity of FLT3 tyrosine kinase in a cell comprising the step of contacting the cell with a FLT3 kinase inhibitor and a farnesyl transferase inhibitor.

The present invention also provides a method for reducing or inhibiting the expression of FLT3 tyrosine kinase in a subject comprising the step of administering a FLT3 kinase inhibitor and a farnesyl transferase inhibitor to the subject.

The present invention further provides a method of inhibiting cell proliferation in a cell comprising the step of contacting the cell with a FLT3 kinase inhibitor and a farnesyl transferase inhibitor.

The kinase activity of FLT3 in a cell or a subject can be determined by procedures well known in the art, such as the FLT3 kinase assay described herein.

The term “subject” as used herein, refers to an animal, preferably a mammal, most preferably a human, who has been the object of treatment, observation or experiment.

The term “contacting” as used herein, refers to the addition of compound to cells such that compound is taken up by the cell.

In other embodiments to this aspect, the present invention provides both prophylactic and therapeutic methods for treating a subject at risk of (or susceptible to) developing a cell proliferative disorder or a disorder related to FLT3.

In one example, the invention provides methods for preventing in a subject a cell proliferative disorder or a disorder related to FLT3, comprising administering to the subject a prophylactically effective amount of (1) a first pharmaceutical composition comprising a FLT3 kinase inhibitor and a pharmaceutically acceptable carrier, and (2) a second pharmaceutical composition comprising a farnesyl transferase inhibitor and a pharmaceutically acceptable carrier.

In one example, the invention provides methods for preventing in a subject a cell proliferative disorder or a disorder related to FLT3, comprising administering to the subject a prophylactically effective amount of a pharmaceutical composition comprising a FLT3 kinase inhibitor, a farnesyl transferase inhibitor and a pharmaceutically acceptable carrier.

Administration of said prophylactic agent(s) can occur prior to the manifestation of symptoms characteristic of the cell proliferative disorder or disorder related to FLT3, such that a disease or disorder is prevented or, alternatively, delayed in its progression.

In another example, the invention pertains to methods of treating in a subject a cell proliferative disorder or a disorder related to FLT3 comprising administering to the subject a therapeutically effective amount of (1) a first pharmaceutical composition comprising a FLT3 kinase inhibitor and a pharmaceutically acceptable carrier, and (2) a second pharmaceutical composition comprising a farnesyl transferase inhibitor and a pharmaceutically acceptable carrier.

In another example, the invention pertains to methods of treating in a subject a cell proliferative disorder or a disorder related to FLT3 comprising administering to the subject a therapeutically effective amount of a pharmaceutical composition comprising a FLT3 kinase inhibitor, a farnesyl transferase inhibitor and a pharmaceutically acceptable carrier.

Administration of said therapeutic agent(s) can occur concurrently with the manifestation of symptoms characteristic of the disorder, such that said therapeutic agent serves as a therapy to compensate for the cell proliferative disorder or disorders related to FLT3.

The FLT3 kinase inhibitor and farnesyl transferase inhibitor can be administered as a unitary pharmaceutical composition comprising a FLT3 kinase inhibitor, a farnesyl transferase inhibitor and a pharmaceutically acceptable carrier, or as separate pharmaceutical compositions: (1) a first pharmaceutical composition comprising a FLT3 kinase inhibitor and a pharmaceutically acceptable carrier, and (2) a second pharmaceutical composition comprising a farnesyl transferase inhibitor and a pharmaceutically acceptable carrier. In the latter case, the two pharmaceutical compositions may be administered simultaneously (albeit in separate compositions), sequentially in either order, at approximately the same time, or on separate dosing schedules. On separate dosing schedules, the two compositions are administered within a period and in an amount and manner that is sufficient to ensure that an advantageous or synergistic effect is achieved.

It will be appreciated that the preferred method and order of administration and the respective dosage amounts and regimes for each component of the combination will depend on the agent being administered, their route of administration, the particular tumor being treated and the particular host being treated.

As will be understood by those of ordinary skill in the art, the optimum method and order of administration and the dosage amounts and regime of the FLT3 kinase inhibitor and farnesyl transferase inhibitor can be readily determined by those skilled in the art using conventional methods and in view of the information set out herein.

Generally, the dosage amounts and regime of the FLT3 kinase inhibitor and farnesyl transferase inhibitor will be similar to or less than those already employed in clinical therapies where these agents are administered alone, or in combination with other chemotherapeutics.

The term “prophylactically effective amount” refers to an amount of an active compound or pharmaceutical agent that inhibits or delays in a subject the onset of a disorder as being sought by a researcher, veterinarian, medical doctor or other clinician.

The term “therapeutically effective amount” as used herein, refers to an amount of active compound or pharmaceutical agent that elicits the biological or medicinal response in a subject that is being sought by a researcher, veterinarian, medical doctor or other clinician, which includes alleviation of the symptoms of the disease or disorder being treated.

Methods are known in the art for determining therapeutically and prophylactically effective doses for the instant pharmaceutical composition(s).

As used herein, the term “composition” is intended to encompass a product comprising the specified ingredients in the specified amounts, as well as any product which results, directly or indirectly, from combinations of the specified ingredients in the specified amounts.

As used herein, the terms “disorders related to FLT3”, or “disorders related to FLT3 receptor”, or “disorders related to FLT3 receptor tyrosine kinase” shall include diseases associated with or implicating FLT3 activity, for example, the overactivity of FLT3, and conditions that accompany with these diseases. The term “overactivity of FLT3” refers to either 1) FLT3 expression in cells which normally do not express FLT3; 2) FLT3 expression by cells which normally do not express FLT3; 3) increased FLT3 expression leading to unwanted cell proliferation; or 4) mutations leading to constitutive activation of FLT3. Examples of “disorders related to FLT3” include disorders resulting from over stimulation of FLT3 due to abnormally high amount of FLT3 or mutations in FLT3, or disorders resulting from abnormally high amount of FLT3 activity due to abnormally high amount of FLT3 or mutations in FLT3. It is known that overactivity of FLT3 has been implicated in the pathogenesis of a number of diseases, including the cell proliferative disorders, neoplastic disorders and cancers listed below.

The term “cell proliferative disorders” refers to unwanted cell proliferation of one or more subset of cells in a multicellular organism resulting in harm (i.e., discomfort or decreased life expectancy) to the multicellular organisms. Cell proliferative disorders can occur in different types of animals and humans. For example, as used herein “cell proliferative disorders” include neoplastic disorders and other cell proliferative disorders.

As used herein, a “neoplastic disorder” refers to a tumor resulting from abnormal or uncontrolled cellular growth. Examples of neoplastic disorders include, but are not limited to, hematopoietic disorders such as, for instance, the myeloproliferative disorders, such as thrombocythemia, essential thrombocytosis (ET), angiogenic myeloid metaplasia, myelofibrosis (MF), myelofibrosis with myeloid metaplasia (MMM), chronic idiopathic myelofibrosis (IMF), polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematoglogical malignancies, including myelodysplasia, multiple myeloma, leukemias and lymphomas. Examples of hematological malignancies include, for instance, leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma, (MM).

In a further embodiment to this aspect, the invention encompasses a multiple component therapy for treating or inhibiting onset of a cell proliferative disorder or a disorder related to FLT3 in a subject comprising administering to the subject a therapeutically or prophylactically effective amount of a FLT3 kinase inhibitor, a farnesyl transferase inhibitor and and one or more other anti-cell proliferation therapy(ies) including chemotherapy, radiation therapy, gene therapy and immunotherapy.

As used herein, “chemotherapy” refers to a therapy involving a chemotherapeutic agent. A variety of chemotherapeutic agents may be used in the multiple component treatment methods disclosed herein. Chemotherapeutic agents contemplated as exemplary, include, but are not limited to: platinum compounds (e.g., cisplatin, carboplatin, oxaliplatin); taxane compounds (e.g., paclitaxcel, docetaxol); campotothecin compounds (irinotecan, topotecan); vinca alkaloids (e.g., vincristine, vinblastine, vinorelbine); anti-tumor nucleoside derivatives (e.g., 5-fluorouracil, leucovorin, gemcitabine, capecitabine); alkylating agents (e.g., cyclophosphamide, carmustine, lomustine, thiotepa); epipodophyllotoxins/podophyllotoxins (e.g. etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologics (e.g., gemtuzamab, cetuximab, rituximab, pertuzumab, trastuzumab, bevacizumab, erlotinib); antibiotics/anthracyclines (e.g. idarubicin, actinomycin D, bleomycin, daunorubicin, doxorubicin, mitomycin C, dactinomycin, carminomycin, daunomycin); antimetabolites (e.g., aminopterin, clofarabine, cytosine arabinoside, methotrexate); tubulin-binding agents (e.g. combretastatin, colchicine, nocodazole); topoisomerase inhibitors (e.g., camptothecin). Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. See Simpson W G, The calcium channel blocker verapamil and cancer chemotherapy. Cell Calcium. 1985 December; 6(6):449-67. Additionally, yet to emerge chemotherapeutic agents are contemplated as being useful in combination with the compound of the present invention.

In another embodiment of the present invention, the FLT3 kinase inhibitor and farnesyl transferase inhibitor may be administered in combination with radiation therapy. As used herein, “radiation therapy” refers to a therapy that comprises exposing the subject in need thereof to radiation. Such therapy is known to those skilled in the art. The appropriate scheme of radiation therapy will be similar to those already employed in clinical therapies wherein the radiation therapy is used alone or in combination with other chemotherapeutics.

In another embodiment of the present invention, the FLT3 kinase inhibitor and farnesyl transferase inhibitor may be administered in combination with gene therapy. As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, RNA-based strategies such as ribozymes, RNA decoys, antisense messenger RNAs and small interfering RNA (siRNA) molecules and the so-called ‘suicide genes’.

In other embodiments of this invention, the FLT3 kinase inhibitor and farnesyl transferase inhibitor may be administered in combination with immunotherapy. As used herein, “immunotherapy” refers to a therapy targeting particular protein involved in tumor development via antibodies specific to such protein. For example, monoclonal antibodies against vascular endothelial growth factor have been used in treating cancers.

Where one or more additional chemotherapeutic agent(s) are used in conjunction with the FLT3 kinase inhibitor and farnesyl transferase inhibitor, the additional chemotherapeutic agent(s), the FLT3 kinase inhibitor and the farnesyl transferase inhibitor may be administered simultaneously (e.g. in separate or unitary compositions) sequentially in any order, at approximately the same time, or on separate dosing schedules. In the latter case, the pharmaceuticals will be administered within a period and in an amount and manner that is sufficient to ensure that an advantageous and synergistic effect is achieved. It will be appreciated that the preferred method and order of administration and the respective dosage amounts and regimes for the additional chemotherapeutic agent(s) will depend on the particular chemotherapeutic agent(s) being administered in conjunction with the FLT3 kinase inhibitor and farnesyl transferase inhibitor, their route of administration, the particular tumor being treated and the particular host being treated. As will be understood by those of ordinary skill in the art, the appropriate doses of the additional chemotherapeutic agent(s) will be generally similar to or less than those already employed in clinical therapies wherein the chemotherapeutics are administered alone or in combination with other chemotherapeutics.

The optimum method and order of administration and the dosage amounts and regime can be readily determined by those skilled in the art using conventional methods and in view of the information set out herein.

By way of example only, platinum compounds are advantageously administered in a dosage of 1 to 500 mg per square meter (mg/m²) of body surface area, for example 50 to 400 mg/m², particularly for cisplatin in a dosage of about 75 mg/m² and for carboplatin in about 300 mg/m² per course of treatment. Cisplatin is not absorbed orally and must therefore be delivered via injection intravenously, subcutaneously, intratumorally or intraperitoneally.

By way of example only, taxane compounds are advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m² per course of treatment.

By way of example only, camptothecin compounds are advantageously administered in a dosage of 0.1 to 400 mg per square meter (mg/m²) of body surface area, for example 1 to 300 mg/m², particularly for irinotecan in a dosage of about 100 to 350 mg/m² and for topotecan in about 1 to 2 mg/m² per course of treatment.

By way of example only, vinca alkaloids may be advantageously administered in a dosage of 2 to 30 mg per square meter (mg/m²) of body surface area, particularly for vinblastine in a dosage of about 3 to 12 mg/m², for vincristine in a dosage of about 1 to 2 mg/m², and for vinorelbine in dosage of about 10 to 30 mg/m² per course of treatment.

By way of example only, anti-tumor nucleoside derivatives may be advantageously administered in a dosage of 200 to 2500 mg per square meter (mg/m²) of body surface area, for example 700 to 1500 mg/m². 5-fluorouracil (5-FU) is commonly used via intravenous administration with doses ranging from 200 to 500 mg/m² (preferably from 3 to 15 mg/kg/day). Gemcitabine is advantageously administered in a dosage of about 800 to 1200 mg/m² and capecitabine is advantageously administered in about 1000 to 2500 mg/m² per course of treatment.

By way of example only, alkylating agents may be advantageously administered in a dosage of 100 to 500 mg per square meter (mg/m²) of body surface area, for example 120 to 200 mg/m², particularly for cyclophosphamide in a dosage of about 100 to 500 mg/m², for chlorambucil in a dosage of about 0.1 to 0.2 mg/kg of body weight, for carmustine in a dosage of about 150 to 200 mg/m², and for lomustine in a dosage of about 100 to 150 mg/m² per course of treatment.

By way of example only, podophyllotoxin derivatives may be advantageously administered in a dosage of 30 to 300 mg per square meter (mg/m2) of body surface area, for example 50 to 250 mg/m², particularly for etoposide in a dosage of about 35 to 100 mg/m² and for teniposide in about 50 to 250 mg/m² per course of treatment.

By way of example only, anthracycline derivatives may be advantageously administered in a dosage of 10 to 75 mg per square meter (mg/m²) of body surface area, for example 15 to 60 mg/m², particularly for doxorubicin in a dosage of about 40 to 75 mg/m², for daunorubicin in a dosage of about 25 to 45 mg/m², and for idarubicin in a dosage of about 10 to 15 mg/m² per course of treatment.

By way of example only, anti-estrogen compounds may be advantageously administered in a dosage of about 1 to 100 mg daily depending on the particular agent and the condition being treated. Tamoxifen is advantageously administered orally in a dosage of 5 to 50 mg, preferably 10 to 20 mg twice a day, continuing the therapy for sufficient time to achieve and maintain a therapeutic effect. Toremifene is advantageously administered orally in a dosage of about 60 mg once a day, continuing the therapy for sufficient time to achieve and maintain a therapeutic effect. Anastrozole is advantageously administered orally in a dosage of about 1 mg once a day. Droloxifene is advantageously administered orally in a dosage of about 20-100 mg once a day. Raloxifene is advantageously administered orally in a dosage of about 60 mg once a day. Exemestane is advantageously administered orally in a dosage of about 25 mg once a day.

By way of example only, biologics may be advantageously administered in a dosage of about 1 to 5 mg per square meter (mg/m²) of body surface area, or as known in the art, if different. For example, trastuzumab is advantageously administered in a dosage of 1 to 5 mg/m² particularly 2 to 4 mg/m² per course of treatment.

Dosages may be administered, for example once, twice or more per course of treatment, which may be repeated for example every 7, 14, 21 or 28 days.

The FLT3 kinase inhibitor and farnesyl transferase inhibitor can be administered to a subject systemically, for example, intravenously, orally, subcutaneously, intramuscular, intradermal, or parenterally. The FLT3 kinase inhibitor and farnesyl transferase inhibitor can also be administered to a subject locally. Non-limiting examples of local delivery systems include the use of intraluminal medical devices that include intravascular drug delivery catheters, wires, pharmacological stents and endoluminal paving. The FLT3 kinase inhibitor and farnesyl transferase inhibitor can further be administered to a subject in combination with a targeting agent to achieve high local concentration of the FLT3 kinase inhibitor and farnesyl transferase inhibitor at the target site. In addition, the FLT3 kinase inhibitor and farnesyl transferase inhibitor may be formulated for fast-release or slow-release with the objective of maintaining the drugs or agents in contact with target tissues for a period ranging from hours to weeks.

The separate pharmaceutical compositions comprising the FLT3 kinase inhibitor in association with a pharmaceutically acceptable carrier, and the farnesyl transferase inhibitor in association with a pharmaceutically acceptable carrier may contain between about 0.1 mg and 1000 mg, preferably about 100 to 500 mg, of the individual agents compound, and may be constituted into any form suitable for the mode of administration selected.

The unitary pharmaceutical composition comprising the FLT3 kinase inhibitor and farnesyl transferase inhibitor in association with a pharmaceutically acceptable carrier may contain between about 0.1 mg and 1000 mg, preferably about 100 to 500 mg, of the compound, and may be constituted into any form suitable for the mode of administration selected.

The phrases “pharmaceutically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

The pharmaceutical compositions of the present invention, whether unitary or separate, may be formulated for slow release of the FLT3 kinase inhibitor and farnesyl transferase inhibitor. Such a composition, unitary or separate, includes a slow release carrier (typically, a polymeric carrier) and one, or in the case of the unitary composition, both, of the FLT3 kinase inhibitor and farnesyl transferase inhibitor.

Slow release biodegradable carriers are well known in the art. These are materials that may form particles that capture therein an active compound(s) and slowly degrade/dissolve under a suitable environment (e.g., aqueous, acidic, basic, etc) and thereby degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter).

Farnesyltransferase Inhibitors

Examples of farnesyltransferase inhibitors which may be employed in the methods or treatments in accordance with the present invention include the farnesyltransferase inhibitors (“FTIs”) of formula (I), (II), (III), (IV), (V), (VI), (VII), (VIII) or (IX) above.

Preferred FTIs include compounds of formula (I), (II) or (III): 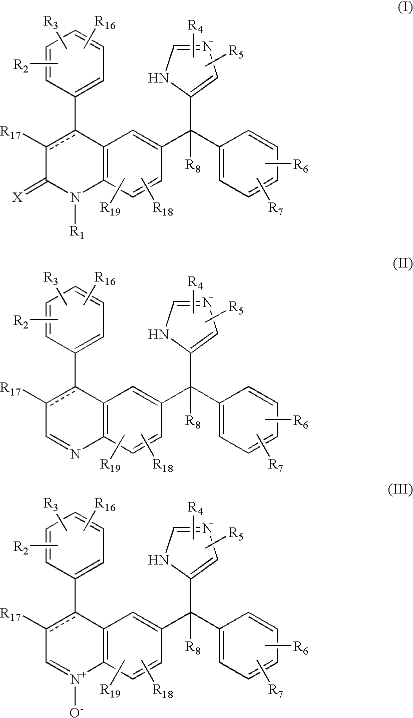
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
   wherein
      Alk¹ is C₁₋₆alkanediyl,
      R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula
   —O—CH₂—O— (a-1),
   —O—CH₂—CH₂—O— (a-2),
   —O—CH═CH— (a-3),
   —O—CH₂—CH₂— (a-4),
   —O—CH₂—CH₂—CH₂— (a-5), or
   —CH═CH—CH═CH— (a-6);
R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or
when on adjacent positions R⁶ and R⁷ taken together may form a bivalent radical of formula
   —O—CH₂—O— (c-1), or
   —CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula
   —O—R¹⁰ (b-1),
   —S—R¹⁰ (b-2),
   —N—R¹¹R¹² (b-3),
   wherein
      R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical of formula -Alk²-OR¹³ or -Alk²-NR¹⁴R¹⁵;
      R¹¹ is hydrogen, C₁₋₁₂alkyl, Ar¹ or Ar²C₁₋₆alkyl;
      R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
      or a radical of formula -Alk²-OR¹³ or -Alk²-NR¹⁴R¹⁵;
      wherein
         Alk² is C₁₋₆alkanediyl;
         R¹³ is hydrogen, C₁₋₆alkyl, C₁₋₆alkylcarbonyl, hydroxyC₁₋₆alkyl, Ar¹ or Ar²C₁₋₆alkyl;
         R¹⁴ is hydrogen, C₁₋₆alkyl, Ar¹ or Ar²C₁₋₆alkyl;
         R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

In Formulas (I), (II) and (III), R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl, C₁₋₆alkylS(O)₂C₁₋₆alkyl.

Preferably the substituent R¹⁸ in Formulas (I), (II) and (III) is situated on the 5 or 7 position of the quinolinone moiety and substituent R¹⁹ is situated on the 8 position when R¹⁸ is on the 7-position.

Preferred examples of FTIs are those compounds of formula (I) wherein X is oxygen.

Also, examples of preferred FTIs are those compounds of formula (I) wherein the dotted line represents a bond, so as to form a double bond.

Another group of preferred FTIs are those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl.

Still another group of preferred FTIs are those compounds of formula (I) wherein R³ is hydrogen or halo; and R² is halo, C₁₋₆alkyl, C₂₋₆alkenyl, C₁₋₆alkyloxy, trihalomethoxy or hydroxyC₁₋₆alkyloxy.

A further group of preferred FTIs are those compounds of formula (I) wherein R² and R³ are on adjacent positions and taken together to form a bivalent radical of formula (a-1), (a-2) or (a-3).

A still further group of preferred FTIs are those compounds of formula (I) wherein R⁵ is hydrogen and R⁴ is hydrogen or C₁₋₆alkyl.

Yet another group of preferred FTIs are those compounds of formula (I) wherein R⁷ is hydrogen; and R⁶ is C₁₋₆alkyl or halo, preferably chloro, especially 4-chloro.

Another exemplary group of preferred FTIs are those compounds of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, hydroxy, C₁₋₆alkyloxyC₁₋₆alkylcarbonyl, or a radical of formula -Alk²-OR¹³ wherein R¹³ is hydrogen or C₁₋₆alkyl.

Preferred compounds are also those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl; R² is halo, C₁₋₆alkyl, C₂₋₆alkenyl, C₁₋₆alkyloxy, trihalomethoxy, hydroxyC₁₋₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, or a radical of formula -Alk²-OR¹³ wherein R¹³ is C₁₋₆alkyl; R¹⁷ is hydrogen and R¹⁸ is hydrogen.

Especially preferred FTIs are:
4-(3-chlorophenyl)-6-[(4-chlorophenyl)hydroxy(1-methyl-1H-imidazol-5-yl)methyl]-1-methyl-2(1H)-quinolinone;
6-[amino(4-chlorophenyl)-1-methyl-1H-imidazol-5-ylmethyl]-4-(3-chlorophenyl)-1-methyl-2(1H)-quinolinone;
6-[(4-chlorophenyl)hydroxy(1-methyl-1H-imidazol-5-yl)methyl]-4-(3-ethoxyphenyl)-1-methyl-2(1H)-quinolinone;
6-[(4-chlorophenyl)(1-methyl-1H-imidazol-5-yl)methyl]-4-(3-ethoxyphenyl)-1-methyl-2(1H)-quinolinone monohydrochloride.monohydrate;
6-[amino(4-chlorophenyl)(1-methyl-1H-imidazol-5-yl)methyl]-4-(3-ethoxyphenyl)-1-methyl-2(1H)-quinolinone;
6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salt; and
(+)-6-[amino(4-chlorophenyl)(1-methyl-1H-imidazol-5-yl)methyl]-4-(3-chlorophenyl)-1-methyl-2(1H)-quinolinone (tipifarnib; Compound 75 in Table 1 of WO 97/21701); and the pharmaceutically acceptable acid addition salts and the stereochemically isomeric forms thereof.

Tipifarnib or ZARNESTRA® is an especially preferred FTI.

Further preferred FTIs include compounds of formula (IX) wherein one or more of the following apply:
═X¹—X²—X³ is a trivalent radical of formula (x-1), (x-2), (x-3), (x-4) or (x-9) wherein each R⁶ independently is hydrogen, C₁₋₄alkyl, C₁₋₄alkyloxycarbonyl, amino or aryl and R⁷ is hydrogen;
>Y¹—Y²— is a trivalent radical of formula (y-1), (y-2), (y-3), or (y-4) wherein each R⁹ independently is hydrogen, halo, carboxyl, C₁₋₄alkyl or C₁₋₄alkyloxycarbonyl;
r is 0, 1 or 2;
s is 0 or 1;
t is 0;
R¹ is halo, C₁₋₆alkyl or two R¹ substituents ortho to one another on the phenyl ring may independently form together a bivalent radical of formula (a-1);
R² is halo;
R³ is halo or a radical of formula (b-1) or (b-3) wherein
   R¹⁰ is hydrogen or a radical of formula -Alk-OR¹³.
   R¹¹ is hydrogen;
R¹² is hydrogen, C₁₋₆alkyl, C₁₋₆alkylcarbonyl, hydroxy, C₁₋₆alkyloxy or mono- or di(C₁₋₆alkyl)aminoC₁₋₆alkylcarbonyl;
   Alk is C₁₋₆alkanediyl and R¹³ is hydrogen;
   R⁴ is a radical of formula (c-1) or (c-2) wherein
      R¹⁶ is hydrogen, halo or mono- or di(C₁₋₄alkyl)amino;
      R¹⁷ is hydrogen or C₁₋₆alkyl;
   aryl is phenyl.

Another group of preferred FTIs are compounds of formula (IX) wherein ═X¹—X²—X³ is a trivalent radical of formula (x-1), (x-2), (x-3), (x-4) or (x-9), >Y1-Y2 is a trivalent radical of formula (y-2), (y-3) or (y-4), r is 0 or 1, s is 1, t is 0, R¹ is halo, C₍₁₋₄₎alkyl or forms a bivalent radical of formula (a-1), R² is halo or C₁₋₄alkyl, R³ is hydrogen or a radical of formula (b-1) or (b-3), R⁴ is a radical of formula (c-1) or (c-2), R⁶ is hydrogen, C₁₋₄alkyl or phenyl, R⁷ is hydrogen, R⁹ is hydrogen or C₁₋₄alkyl, R¹⁰ is hydrogen or -Alk-OR¹³, R¹¹ is hydrogen and R¹² is hydrogen or C₁₋₆alkylcarbonyl and R¹³ is hydrogen;

Preferred FTIs are those compounds of formula (IX) wherein ═X¹—X²—X³ is a trivalent radical of formula (x-1) or (x-4), >Y1-Y2 is a trivalent radical of formula (y-4), r is 0 or 1, s is 1, t is 0, R¹ is halo, preferably chloro and most preferably 3-chloro, R² is halo, preferably 4-chloro or 4-fluoro, R³ is hydrogen or a radical of formula (b-1) or (b-3), R⁴ is a radical of formula (c-1) or (c-2), R⁶ is hydrogen, R⁷ is hydrogen, R⁹ is hydrogen, R¹⁰ is hydrogen, R¹¹ is hydrogen and R¹² is hydrogen.

Other preferred FTIs are those compounds of formula (IX) wherein ═X¹—X²—X³ is a trivalent radical of formula (x-2), (x-3) or (x-4), >Y1-Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy.

Especially preferred FTI compounds of formula (IX) are:
7-[(4-fluorophenyl)(1H-imidazol-1-yl)methyl]-5-phenylimidazo[1,2-a]quinoline;
α-(4-chlorophenyl)-α-(1-methyl-1H-imidazol-5-yl)-5-phenylimidazo[1,2-a]quinoline-7-methanol;
5-(3-chlorophenyl)-α-(4-chlorophenyl)-α-(1-methyl-1H-imidazol-5-yl)-imidazo[1,2-a]quinoline-7-methanol;
5-(3-chlorophenyl)-α-(4-chlorophenyl)-α-(1-methyl-1H-imidazol-5-yl)imidazo[1,2-a]quinoline-7-methanamine;
5-(3-chlorophenyl)-α-(4-chlorophenyl)-α-(1-methyl-1H-imidazol-5-yl)tetrazolo[1,5-a]quinoline-7-methanamine;
5-(3-chlorophenyl)-α-(4-chlorophenyl)-1-methyl-α-(1-methyl-1H-imidazol-5-yl)-1,2,4-triazolo[4,3-a]quinoline-7-methanol;
5-(3-chlorophenyl)-α-(4-chlorophenyl)-α-(1-methyl-1H-imidazol-5-yl)tetrazolo[1,5-a]quinoline-7-methanamine;
5-(3-chlorophenyl)-α-(4-chlorophenyl)-α-(1-methyl-1H-imidazol-5-yl)tetrazolo[1,5-a]quinazoline-7-methanol;
5-(3-chlorophenyl)-α-(4-chlorophenyl)-4,5-dihydro-α-(1-methyl-1H-imidazol-5-yl)tetrazolo[1,5-a]quinazoline-7-methanol;
5-(3-chlorophenyl)-α-(4-chlorophenyl)-α-(1-methyl-1H-imidazol-5-yl)tetrazolo[1,5-a]quinazoline-7-methanamine;
5-(3-chlorophenyl)-α-(4-chlorophenyl)-N-hydroxy-α-(1-methyl-1H-imidazol-5-yl)tetrahydro[1,5-a]quinoline-7-methanamine; and
α-(4-chlorophenyl)-α-(1-methyl-1H-imidazol-5-yl)-5-(3-methylphenyl)tetrazolo[1,5-a]quinoline-7-methanamine; and the pharmaceutically acceptable acid addition salts and the stereochemically isomeric forms thereof.

5-(3-chlorophenyl)-α-(4-chlorophenyl)-α-(1-methyl-1H-imidazol-5-yl)tetrazolo[1,5-a]quinazoline-7-methanamine, especially the (−) enantiomer, and its pharmaceutically acceptable acid addition salts is an especially preferred FTI.

The pharmaceutically acceptable acid or base addition salts as mentioned hereinabove are meant to comprise the therapeutically active non-toxic acid and non-toxic base addition salt forms which the FTI compounds of formulas (I), (II), (III), (IV), (V), (VI), (VII), (VIII) or (IX) are able to form. The FTI compounds of formulas (I), (II), (III), (IV), (V), (VI), (VII), (VIII) or (IX) which have basic properties can be converted in their pharmaceutically acceptable acid addition salts by treating the base form with an appropriate acid. Appropriate acids include, for example, inorganic acids such as hydrohalic acids, e.g. hydrochloric or hydrobromic acid; sulfuric; nitric; phosphoric and the like acids; or organic acids, such as acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The FTI compounds of formulae (I), (II), (III), (IV), (V), (VI), (VII), (VIII) or (IX) which have acidic properties may be converted in their pharmaceutically acceptable base addition salts by treating the acid form with a suitable organic or inorganic base.

Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids, for example, arginine, lysine and the like.

Acid and base addition salts also comprise the hydrates and the solvent addition forms which the preferred FTI compounds of formulae (I), (II), (III), (IV), (V), (VI), (VII), (VIII) or (IX) are able to form. Examples of such forms are e.g. hydrates, alcoholates and the like.

The FTI compounds of formulae (I), (II), (III), (IV), (V), (VI), (VII), (VIII) or (IX), as used hereinbefore, encompass all stereochemically isomeric forms of the depicted structural formulae (all possible compounds made up of the same atoms bonded by the same sequence of bonds but having different three-dimensional structures that are not interchangeable). Unless otherwise mentioned or indicated, the chemical designation of an FTI compound should be understood as encompassing the mixture of all possible stereochemically isomeric forms which the compound may possess. Such mixture may contain all diastereomers and/or enantiomers of the basic molecular structure of the compound. All stereochemically isomeric forms of the FTI compounds of formulae (I), (II), (III), (IV), (V), (VI), (VII), (VIII) or (IX) both in pure form or in admixture with each other are intended to be embraced within the scope of the depicted formulae.

Some of the FTI compounds of formulae (I), (II), (III), (IV), (V), (VI), (VII), (VIII) or (IX) may also exist in their tautomeric forms. Such forms, although not explicitly shown in the above formulae, are intended to be included within the scope thereof.

Thus, unless indicated otherwise hereinafter, the terms “compounds of formulae (I), (II), (III), (IV), (V), (VI), (VII), (VIII) or (IX)” and “farnesyltransferase inhibitors of formulae (I), (II), (III), (IV), (V), (VI), (VII), (VIII) or (IX)” are meant to include also the pharmaceutically acceptable acid or base addition salts and all stereoisomeric and tautomeric forms.

Other farnesyltransferase inhibitors which can be employed in accordance with the present invention include: Arglabin, perrilyl alcohol, SCH-66336, 2(S)-[2(S)-[2(R)-amino-3-mercapto]propylamino-3 (S)-methyl]-pentyloxy-3-phenylpropionyl-methionine sulfone (Merck); L778123, BMS 214662, Pfizer compounds A and B described above. Suitable dosages or therapeutically effective amounts for the compounds Arglabin (WO98/28303), perrilyl alcohol (WO 99/45712), SCH-66336 (U.S. Pat. No. 5,874,442), L778123 (WO 00/01691), 2(S)-[2(S)-[2(R)-amino-3-mercapto]propylamino-3 (S)-methyl]-pentyloxy-3-phenylpropionyl-methionine sulfone (WO94/10138), BMS 214662 (WO 97/30992), Pfizer compounds A and B (WO 00/12499 and WO 00/12498) are given in the published patent specifications or are known to or can be readily determined by a person skilled in the art.

FLT3 Kinase Inhibitors

The FLT3 kinase inhibitors of the present invention comprise compounds selected from the group consisting of Formula I′ and Formula II′: 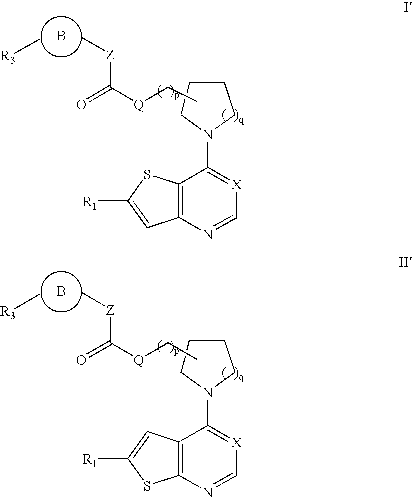
and N-oxides, pharmaceutically acceptable salts, and stereochemical isomers thereof, wherein:
q is 0, 1 or 2;
p is 0 or 1;
Q is NH, N(alkyl), O, or a direct bond;
X is N or CH;
Z is NH, N(alkyl), or CH₂;
B is aryl (wherein said aryl is preferably phenyl), cyclopentadienyl, cycloalkyl (wherein said cycloalkyl is preferably cyclopentanyl, cyclohexanyl, cyclopentenyl or cyclohexenyl), heteroaryl (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or a nine to ten membered benzo-fused heteroaryl (wherein said nine to ten membered benzo-fused heteroaryl is preferably benzothiazolyl, benzooxazolyl, benzoimidazolyl, benzofuranyl, indolyl, quinolinyl, isoquinolinyl, or benzo[b]thiophenyl);
R₁ is: 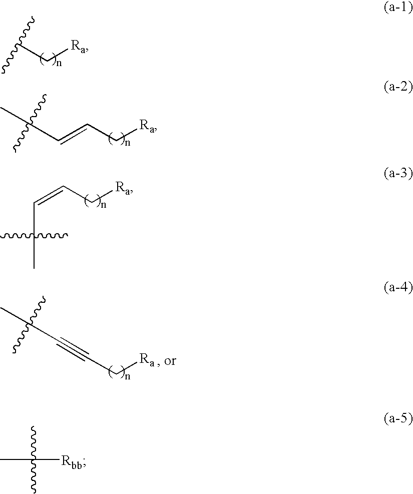
wherein
n is 1, 2, 3 or 4;
Rₐ is hydrogen, heteroaryl optionally substituted with R₅ (wherein said heteroaryl is preferably pyrrolyl, furanyl, thiophenyl, imidazolyl, thiazolyl, oxazolyl, pyranyl, thiopyranyl, pyridinyl, pyrimidinyl, triazolyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, triazolyl, or pyrazinyl), hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅ (wherein said heterocyclyl is preferably pyrrolidinyl, tetrahydrofuranyl, tetrahydrothiophenyl, imidazolidinyl, thiazolidinyl, oxazolidinyl, tetrahydropyranyl, tetrahydrothiopyranyl, thiomorphlinyl, thiomorpholinyl-1,1-dioxide, piperidinyl, morpholinyl or piperazinyl), —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl (wherein the aryl portion of said aralkyl is preferrably phenyl), or heteroaralkyl (wherein the heteroaryl portion of said heteroaralkyl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or R_{w} and Rₓ may optionally be taken together to form a 5 to 7 membered ring, optionally containing a heteromoiety selected from: O, NH, N(alkyl), SO₂, SO, or S, preferably selected from the group consisting of: 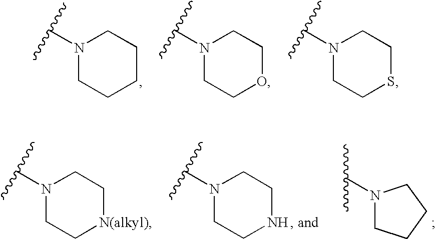
R_{y} is selected from: hydrogen, alkyl, alkenyl, cycloalkyl (wherein said cycloalkyl is preferably cyclopentanyl or cyclohexanyl), aryl (wherein said aryl is preferably phenyl), aralkyl (wherein the aryl portion of said aralkyl is preferably phenyl), heteroaralkyl (wherein the heteroaryl portion of said heteroaralkyl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl), or heteroaryl (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide, and most preferably pyrrolyl, furanyl, thiophenyl, imidazolyl, thiazolyl, oxazolyl, pyridinyl, pyrimidinyl, or pyrazinyl); and
   R₃ is one or more substituents, optionally present, and independently selected from: alkyl, alkoxy, halogen, alkoxyether, hydroxyl, thio, nitro, cycloalkyl optionally substituted with R₄ (wherein said cycloalkyl is preferably cyclopentanyl or cyclohexanyl), heteroaryl optionally substituted with R₄ (wherein said heteroaryl is preferably pyrrolyl, furanyl, thiophenyl, imidazolyl, thiazolyl, oxazolyl, pyranyl, thiopyranyl, pyridinyl, pyrimidinyl, pyrazinyl, pyridinyl-N-oxide, or pyrrolyl-N-oxide; and most preferably pyrrolyl, furanyl, thiophenyl, imidazolyl, thiazolyl, oxazolyl, pyridinyl, pyrimidinyl, or pyrazinyl), alkylamino, heterocyclyl optionally substituted with R₄ (wherein said heterocyclyl is preferably azapenyl, pyrrolidinyl, tetrahydrofuranyl, tetrahydrothiophenyl, imidazolidinyl, thiazolidinyl, oxazolidinyl, tetrahydropyranyl, tetrahydrothiopyranyl, piperidinyl, morpholinyl, or piperazinyl), partially unsaturated heterocyclyl optionally substituted with R₄, (wherein said partially unsaturated heterocyclyl is preferably tetrahydropyridinyl. tetrahydropyrazinyl, dihydrofuranyl, dihydrooxazinyl, dihydropyrrolyl, or dihydroimidazolyl), —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

As used hereafter, the terms “compounds of Formula I′”, “compounds of Formula II′” and “Compounds of Formula I′ and Formula II′” are meant to include also the N-oxides, pharmaceutically acceptable salts, solvates, and stereochemical isomers thereof.

FLT3 Inhibitors of Formula I′—Abbreviations & Definitions

As used in regards to the FLT3 inhibitors of Formula I′ and Formula II′, the following terms are intended to have the following meanings:
ATP adenosine triphosphate
Boc tert-butoxycarbonyl
DCM dichloromethane
DMF dimethylformamide
DMSO dimethylsulfoxide
DIEA diisopropylethylamine
DTT dithiothreitol
EDC 1-(3-dimethylaminopropyl)-3-ethylcarbodiimide hydrochloride
EDTA ethylenediaminetetraaceticacid
EtOAc ethyl acetate
FBS fetal bovine serum
FP fluorescence polarization
GM-CSF granulocyte and macrophage colony stimulating factor
HBTU O-benzotriazol-1-yl-N,N,N′,N′-tetramethyluronium hexafluorophosphate
Hex hexane
HOBT 1-hydroxybenzotriazole hydrate
HPβCD hydroxypropyl β-cyclodextrin
HRP horseradish peroxidase
i-PrOH isopropyl alcohol
LC/MS (ESI) Liquid chromatography/mass spectrum (electrospray ionization)
MeOH Methyl alcohol
NMM N-methylmorpholine
NMR nuclear magnetic resonance
PS polystyrene
PBS phosphate buffered saline
RPMI Rosewell Park Memorial Institute
RT room temperature
RTK receptor tyrosine kinase
NaHMDS sodium hexamethyldisilazane
SDS-PAGE sodium dodecyl sulfate polyacrylamide gel electrophoreisis
TEA triethylamine
TFA trifluoroacetic acid
THF tetrahydrofuran
TLC thin layer chromatography
   (Additional abbreviations are provided where needed throughout the Specification.)
   Definitions

As used in regards to the FLT3 inhibitors of Formula I′ and Formula II′, the following terms are intended to have the following meanings (additional definitions are provided where needed throughout the Specification):

The term “alkenyl,” whether used alone or as part of a substituent group, for example, “C₁₋₄alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl(2-propenyl), butenyl and the like. Examples include C₂₋₈alkenyl or C₂₋₄alkenyl groups.

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “alkylamino” refers to a radical formed by the removal of one hydrogen atom from the nitrogen of an alkylamine, such as butylamine, and the term “dialkylamino” refers to a radical formed by the removal of one hydrogen atom from the nitrogen of a secondary amine, such as dibutylamine. In both cases it is expected that the point of attachment to the rest of the molecule is the nitrogen atom.

The term “alkynyl,” whether used alone or as part of a substituent group, refers to a partially unsaturated branched or straight chain monovalent hydrocarbon radical having at least one carbon-carbon triple bond, whereby the triple bond is derived by the removal of two hydrogen atoms from each of two adjacent carbon atoms of a parent alkyl molecule and the radical is derived by the removal of one hydrogen atom from a single carbon atom. Typical alkynyl radicals include ethynyl, propynyl, butynyl and the like. Examples include C₂₋₈alkynyl or C₂₋₄alkynyl groups.

The term “alkoxy” refers to a saturated or partially unsaturated branched or straight chain monovalent hydrocarbon alcohol radical derived by the removal of the hydrogen atom from the hydroxide oxygen substituent on a parent alkane, alkene or alkyne. Where specific levels of saturation are intended, the nomenclature “alkoxy”, “alkenyloxy” and “alkynyloxy” are used consistent with the definitions of alkyl, alkenyl and alkynyl. Examples include C₁₋₈alkoxy or C₁₋₄alkoxy groups.

The term “alkoxyether” refers to a saturated branched or straight chain monovalent hydrocarbon alcohol radical derived by the removal of the hydrogen atom from the hydroxide oxygen substituent on a hydroxyether. Examples include 1-hydroxyl-2-methoxy-ethane and 1-(2-hydroxyl-ethoxy)-2-methoxy-ethane groups.

The term “aralkyl” refers to a C₁₋₆ alkyl group containing an aryl substituent. Examples include benzyl, phenylethyl or 2-naphthylmethyl. It is intended that the point of attachment to the rest of the molecule be the alkyl group.

The term “aromatic” refers to a cyclic hydrocarbon ring system having an unsaturated, conjugated π electron system.

The term “aryl” refers to an aromatic cyclic hydrocarbon ring radical derived by the removal of one hydrogen atom from a single carbon atom of the ring system. Typical aryl radicals include phenyl, naphthalenyl, fluorenyl, indenyl, azulenyl, anthracenyl and the like.

The term “arylamino” refers to an amino group, such as ammonia, substituted with an aryl group, such as phenyl. It is expected that the point of attachment to the rest of the molecule is through the nitrogen atom.

The term “benzo-fused cycloalkyl” refers to a bicyclic fused ring system radical wherein one of the rings is phenyl and the other is a cycloalkyl or cycloalkenyl ring. Typical benzo-fused cycloalkyl radicals include indanyl, 1,2,3,4-tetrahydro-naphthalenyl, 6,7,8,9,-tetrahydro-5H-benzocycloheptenyl, 5,6,7,8,9,10-hexahydrobenzocyclooctenyl and the like. A benzo-fused cycloalkyl ring system is a subset of the aryl group.

The term “benzo-fused heteroaryl” refers to a bicyclic fused ring system radical wherein one of the rings is phenyl and the other is a heteroaryl ring. Typical benzo-fused heteroaryl radicals include indolyl, indolinyl, isoindolyl, benzo[b]furyl, benzo[b]thienyl, indazolyl, benzthiazolyl, quinolinyl, isoquinolinyl, cinnolinyl, phthalazinyl, quinazolinyl, and the like. A benzo-fused heteroaryl ring is a subset of the heteroaryl group.

The term “benzo-fused heterocyclyl” refers to a bicyclic fused ring system radical wherein one of the rings is phenyl and the other is a heterocyclyl ring. Typical benzo-fused heterocyclyl radicals include 1,3-benzodioxolyl (also known as 1,3-methylenedioxyphenyl), 2,3-dihydro-1,4-benzodioxinyl (also known as 1,4-ethylenedioxyphenyl), benzo-dihydro-furyl, benzo-tetrahydro-pyranyl, benzo-dihydro-thienyl and the like.

The term “carboxyalkyl” refers to an alkylated carboxy group such as tert-butoxycarbonyl, in which the point of attachment to the rest of the molecule is the carbonyl group.

The term “cyclic heterodionyl” refers to a heterocyclic compound bearing two carbonyl substituents. Examples include thiazolidine dionyls, oxazolidine dionyls and pyrrolidine dionyls.

The term “cycloalkenyl” refers to a partially unsaturated cycloalkyl radical derived by the removal of one hydrogen atom from a hydrocarbon ring system that contains at least one carbon-carbon double bond. Examples include cyclohexenyl, cyclopentenyl and 1,2,5,6-cyclooctadienyl.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₃₋₈cycloalkyl, C₅₋₈cycloalkyl, C₃₋₁₂cycloalkyl, C₃₋₂₀cycloalkyl, decahydronaphthalenyl, and 2,3,4,5,6,7-hexahydro-1H-indenyl.

The term “fused ring system” refers to a bicyclic molecule in which two adjacent atoms are present in each of the two cyclic moieties. Heteroatoms may optionally be present. Examples include benzothiazole, 1,3-benzodioxole and decahydronaphthalene.

The term “hetero” used as a prefix for a ring system refers to the replacement of at least one ring carbon atom with one or more atoms independently selected from N, S, O or P. Examples include rings wherein 1, 2, 3 or 4 ring members are a nitrogen atom; or, 0, 1, 2 or 3 ring members are nitrogen atoms and 1 member is an oxygen or sulfur atom.

The term “heteroaralkyl” refers to a C₁₋₆ alkyl group containing a heteroaryl substituent. Examples include furylmethyl and pyridylpropyl. It is intended that the point of attachment to the rest of the molecule be the alkyl group.

The term “heteroaryl” refers to a radical derived by the removal of one hydrogen atom from a ring carbon atom of a heteroaromatic ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “heteroaryl-fused cycloalkyl” refers to a bicyclic fused ring system radical wherein one of the rings is cycloalkyl and the other is heteroaryl. Typical heteroaryl-fused cycloalkyl radicals include 5,6,7,8-tetrahydro-4H-cyclohepta(b)thienyl, 5,6,7-trihydro-4H-cyclohexa(b)thienyl, 5,6-dihydro-4H-cyclopenta(b)thienyl and the like.

The term “heterocyclyl” refers to a saturated or partially unsaturated monocyclic ring radical derived by the removal of one hydrogen atom from a single carbon or nitrogen ring atom. Typical heterocyclyl radicals include 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, piperidinyl, 1,4-dioxanyl, morpholinyl, 1,4-dithianyl, thiomorpholinyl, piperazinyl, azepanyl, hexahydro-1,4-diazepinyl and the like.

The term “substituted,” refers to a core molecule on which one or more hydrogen atoms have been replaced with one or more functional radical moieties. Substitution is not limited to a core molecule, but may also occur on a substituent radical, whereby the substituent radical becomes a linking group.

The term “independently selected” refers to one or more substituents selected from a group of substituents, wherein the substituents may be the same or different.

The substituent nomenclature used in the disclosure of the FLT3 inhibitors of Formula I′ and Formula II′ was derived by first indicating the atom having the point of attachment, followed by the linking group atoms toward the terminal chain atom from left to right, substantially as in:
(C₁₋₆)alkylC(O)NH(C₁₋₆)alkyl(Ph)
or by first indicating the terminal chain atom, followed by the linking group atoms toward the atom having the point of attachment, substantially as in:
Ph(C₁₋₆)alkylamido(C₁₋₆)alkyl
either of which refers to a radical of the formula:

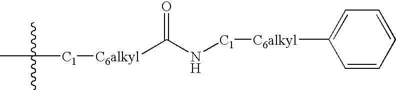

Additionally, lines drawn into ring systems from substituents indicate that the bond may be attached to any of the suitable ring atoms.

When any variable (e.g. R₄) occurs more than one time in any embodiment of the FLT3 inhibitors of Formula I′ and Formula II′, each definition is intended to be independent.

### Embodiments of FLT3 Inhibitors of Formula I′ and Formula II′

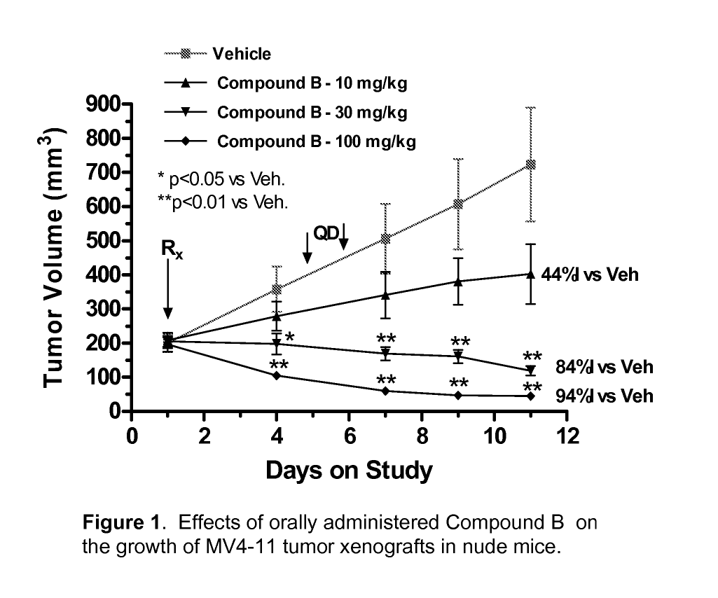

In an embodiment of the FLT3 inhibitors of Formula I′ and Formula II′: N-oxides are optionally present on one or more of: N-1 or N-3 (when X is N) (see FIG. 1 below for ring numbers).

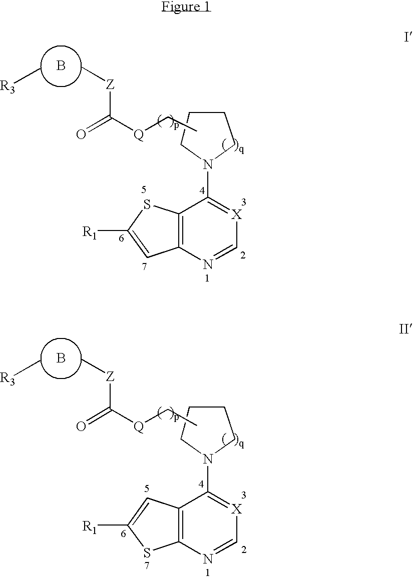

FIG. 1 illustrates ring atoms numbered 1 through 7, as used in the present specification.

In an embodiment of the present invention, the oximine group (—O—N═C—) at postion 5 can be of either the E or the Z configuration.

Preferred embodiments of the the FLT3 inhibitors of Formula I′ and Formula II′ are compounds of Formula I′ and Formula II′ wherein one or more of the following limitations are present:

q is 1 or 2;

p is 0 or 1;

Q is NH, N(alkyl), O, or a direct bond;

X is N;

Z is NH, N(alkyl), or CH₂;

B is aryl or heteroaryl;

R₁ is: 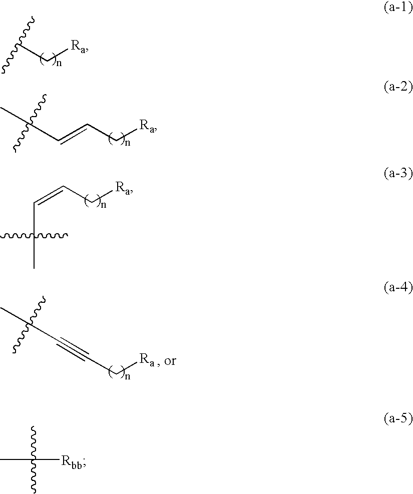
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
   R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Other preferred embodiments of the FLT3 inhibitors of Formula I′ and Formula II′ are compounds of Formula I′ and Formula II′ wherein one or more of the following limitations are present:

q is 1 or 2;

p is 0 or 1;

Q is NH, O, or a direct bond;

X is N;

Z is NH or CH₂;

B is aryl or heteroaryl;

R₁ is: 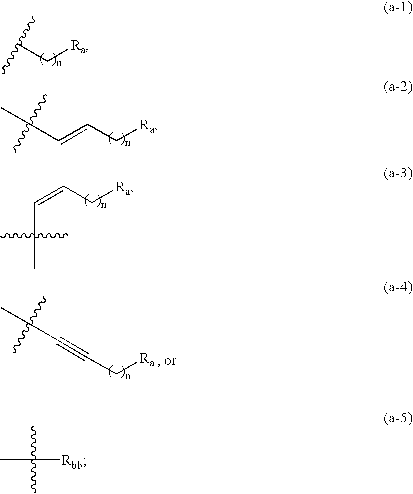
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
   R₃ is one or more substituents, optionally present, and independently selected from: alkyl, alkoxy, halogen, alkoxyether, cycloalkyl optionally substituted with R₄, alkylamino, heterocyclyl optionally substituted with R₄, —O(cycloalkyl), phenoxy optionally substituted with R₄, dialkylamino, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Still other preferred embodiments of the FLT3 inhibitors of Formula I′ and Formula II′ are compounds of Formula I′ and Formula II′ wherein one or more of the following limitations are present:

q is 1 or 2;

p is 0 or 1;

Q is NH, O, or a direct bond;

Z is NH or CH₂;

B is aryl or heteroaryl;

X is N;

R₁ is: 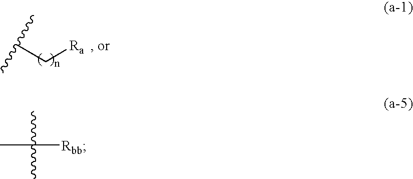
wherein
n is 1, 2, 3 or 4;
Rₐ is hydrogen, hydroxyl, alkylamino, dialkylamino, heterocyclyl optionally substituted with R₅, —CONR_{w}Rₓ, —N(R_{y})CON(R_{w})(Rₓ), —N(R_{w})C(O)ORₓ, —N(R_{w})COR_{y}, —SO₂R_{y}, —NR_{w}SO₂R_{y}, or —NR_{w}SO₂Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
   R₃ is one substituent selected from: alkyl, alkoxy, halogen, alkoxyether, cycloalkyl optionally substituted with R₄, alkylamino, heterocyclyl optionally substituted with R₄, —O(cycloalkyl), phenoxy optionally substituted with R₄, dialkylamino, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

Particularly preferred embodiments of the FLT3 inhibitors of Formula I′ and Formula II′ are compounds of Formula I′ and Formula II′ wherein one or more of the following limitations are present:

q is 1 or 2;

p is 0 or 1;

Q is NH, O, or a direct bond;

Z is NH or CH₂;

B is phenyl or pyridyl;

X is N;

R₁ is: 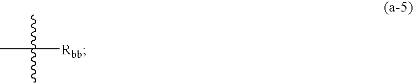
wherein
R_{bb} is hydrogen, halogen, aryl, or heteroaryl; and
   R₃ is one substituent selected from: alkyl, alkoxy, heterocyclyl, —O(cycloalkyl), phenoxy, or dialkylamino.

Most particularly preferred embodiments of the FLT3 inhibitors of Formula I′ and Formula II′ are compounds of Formula I′ and Formula II′ wherein one or more of the following limitations are present:

q is 1 or 2;

p is 0;

Q is NH or O;

Z is NH;

B is phenyl or pyridyl;

X is N;

R₁ is: 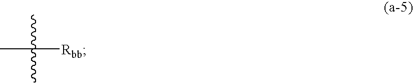
wherein
R_{bb} is hydrogen; and
   R₃ is one substituent selected from: alkyl, —O(cycloalkyl), phenoxy, or dialkylamino.

The FLT3 inhibitors of Formula I′ and Formula II′ may also be present in the form of pharmaceutically acceptable salts.

For use in medicines, the salts of the compounds of the FLT3 inhibitors of Formula I′ and Formula II′ refer to non-toxic “pharmaceutically acceptable salts.” FDA approved pharmaceutically acceptable salt forms (Ref. International J. Pharm. 1986, 33, 201-217; J. Pharm. Sci., 1977, January, 66(1), p1) include pharmaceutically acceptable acidic/anionic or basic/cationic salts.

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine (TEA) or zinc.

The FLT3 inhibitors of the present invention includes within its scope prodrugs of the compounds of Formula I′ and Formula II′. In general, such prodrugs will be functional derivatives of the compounds which are readily convertible in vivo into an active compound. Thus, in the methods of treatment of the present invention, the term “administering” shall encompass the means for treating, ameliorating or preventing a syndrome, disorder or disease described herein with a FLT3 inhibitor of Formula I′ and Formula II′ specifically disclosed or a compound, or prodrug thereof, which would obviously be included within the scope of the invention albeit not specifically disclosed for certain of the instant compounds. Conventional procedures for the selection and preparation of suitable prodrug derivatives are described in, for example, “Design of Prodrugs”, ed. H. Bundgaard, Elsevier, 1985.

One skilled in the art will recognize that the FLT3 inhibitors of Formula I′ and Formula II′ may have one or more asymmetric carbon atoms in their structure. It is intended that the present invention include within its scope single enantiomer forms of the FLT3 inhibitors of Formula I′ and Formula II′, racemic mixtures, and mixtures of enantiomers in which an enantiomeric excess is present.

The term “single enantiomer” as used herein defines all the possible homochiral forms which the compounds of Formula I and their N-oxides, addition salts, quaternary amines or physiologically functional derivatives may possess.

Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (enantiomers).

The term “stereoisomer” refers to isomers of identical constitution that differ in the arrangement of their atoms in space. Enantiomers and diastereomers are examples of stereoisomers.

The term “chiral” refers to the structural characteristic of a molecule that makes it impossible to superimpose it on its mirror image.

The term “enantiomer” refers to one of a pair of molecular species that are mirror images of each other and are not superimposable.

The term “diastereomer” refers to stereoisomers that are not mirror images. The symbols “R” and “S” represent the configuration of substituents around a chiral carbon atom(s).

The term “racemate” or “racemic mixture” refers to a composition composed of equimolar quantities of two enantiomeric species, wherein the composition is devoid of optical activity.

The term “homochiral” refers to a state of enantiomeric purity.

The term “optical activity” refers to the degree to which a homochiral molecule or nonracemic mixture of chiral molecules rotates a plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) configuration, the substituents are on opposite sides in relationship to the carbon-carbon double bond; in the “Z” (same sided) configuration, the substituents are oriented on the same side in relationship to the carbon-carbon double bond. Substituent atoms (other than hydrogen) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” “S,” “E,” “Z,” “cis,” and “trans” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30).

The FLT3 inhibitors of Formula I′ and Formula II′ may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

Furthermore, the FLT3 inhibitors of Formula I′ and Formula II′ may have one or more polymorph or amorphous crystalline forms and as such are intended to be included in the scope of the invention. In addition, some of the FLT3 inhibitors of Formula I′ and Formula II′ may form solvates, for example with water (i.e., hydrates) or common organic solvents. As used herein, the term “solvate” means a physical association of a compound of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope solvates of the FLT3 inhibitors of Formula I′ and Formula II′ of the present invention. Thus, in the methods of treatment of the present invention, the term “administering” shall encompass the means for treating, ameliorating or preventing a syndrome, disorder or disease described herein with a FLT3 inhibitor of Formula I′ and Formula II′ specifically disclosed or a compound, or solvate thereof, which would obviously be included within the scope of the invention albeit not specifically disclosed for certain of the instant compounds.

The FLT3 inhibitors of Formula I′ and Formula II′ may be converted to the corresponding N-oxide forms following art-known procedures for converting a trivalent nitrogen into its N-oxide form. Said N-oxidation reaction may generally be carried out by reacting the starting material of Formula I′ and Formula II′ with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t-butyl hydroperoxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Some of FLT3 inhibitors of Formula I′ and Formula II′ may also exist in their tautomeric forms. Such forms although not explicitly indicated in the present application are intended to be included within the scope of the present invention.

Preparation of FLT3 Inhibitors of Formula I′ and Formula II′

During any of the processes for preparation of the FLT3 inhibitors of Formula I′ and Formula II′, it may be necessary and/or desirable to protect sensitive or reactive groups on any of the molecules concerned. This may be achieved by means of conventional protecting groups, such as those described in Protecting Groups, P. Kocienski, Thieme Medical Publishers, 2000; and T. W. Greene & P. G. M. Wuts, Protective Groups in Organic Synthesis, 3^{rd} ed. Wiley Interscience, 1999. The protecting groups may be removed at a convenient subsequent stage using methods known in the art.

FLT3 inhibitors of Formula I′ and Formula II′ can be prepared by methods known to those who are skilled in the art. The following reaction schemes are only meant to represent examples of the invention and are in no way meant to be a limit of the invention.
General Reaction Scheme

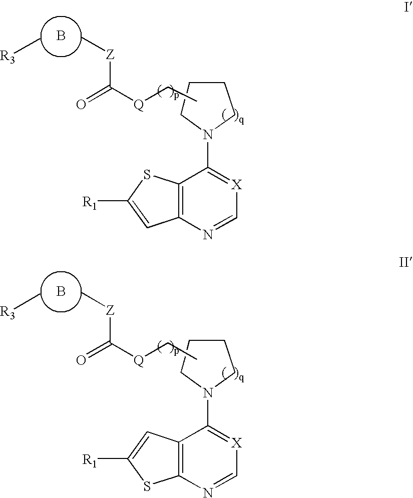

The FLT3 inhibitor compounds of Formula I′ can be prepared by methods known to those who are skilled in the art, wherein Q is O and p, q, B, X, Z, R₁ and R₃ are as defined in Formula I′, may be synthesized as outlined by the general synthetic route illustrated in Scheme 1. Treatment of an appropriate 4-chloro-thieno[3,2-d]pyrimidine or pyridine III′ with an appropriate hydroxy cyclic amine IV′ in a solvent such as isopropanol at a temperature of 50° C. to 150° C. can provide the intermediate V′. Treatment of intermediate V′ with a base such as sodium hydride in a solvent such as tetrahydrofuran (THF) followed by addition of the appropriate acylating group VI′, wherein LG is an appropriate leaving group, such as chloride, p-nitrophenoxy or imidazole, can provide the final product I′. The 4-chloro-thieno[3,2-d]pyrimidines or pyridines III′ are either commercially available or can be prepared by known methods (WO9924440); the hydroxy cyclic amines IV′ are commercially available or can be derived by known methods (JOC, 1961, 26, 1519; EP314362). The acylating reagents VI′ are either commercially available or can be prepared as illustrated in Scheme 1. Treatment of an appropriate R₃BZH, wherein Z is NH or N(alkyl), with an appropriate acylating reagent such as carbonyldiimidazole or p-nitrophenylchloroformate in the presence of a base such as triethylamine can provide VI′. Many R₃BZH reagents are commercially available or can be prepared by a number of known methods (e.g. Tet Lett 1995, 36, 2411-2414). Corrresponding compounds of Formula II′ can be prepared by the same method outlined in Scheme 1 using the appropriate 4-chloro-thieno[2,3-d]pyrimidine or pyridine.

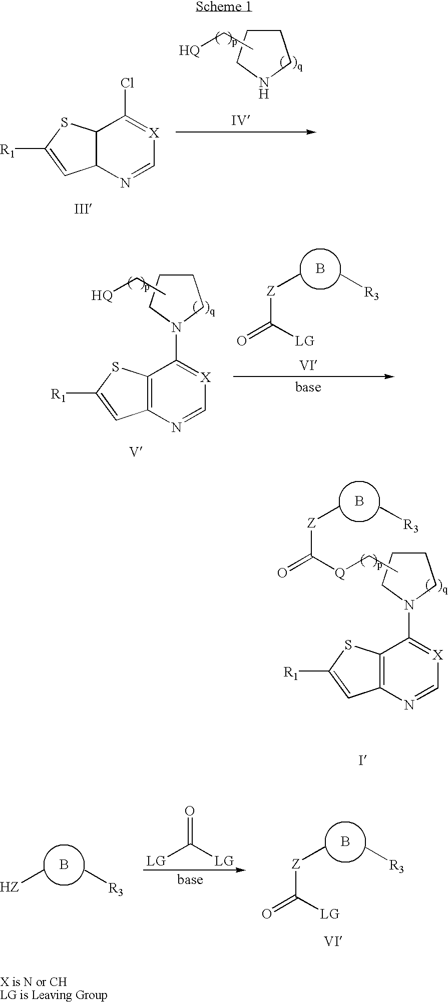

Alternatively FLT3 inhibitor compounds of Formula I′, wherein Q is 0, Z is NH or N(alkyl), and p, q, B, X, R₁ and R₃ are as defined in Formula I′, may be synthesized as outlined by the general synthetic route illustrated in Scheme 2. Treatment of alcohol intermediate V′, prepared as described in Scheme 1, with an acylating agent such as carbonyldiimidazole or p-nitrophenylchloroformate, wherein LG may be chloride, p-nitrophenoxy or imidazole, can provide the acylated intermediate VII′. Treatment of VII′ with an appropriate R₃BZH, wherein Z is NH or N(alkyl), can provide the final product I′. Corresponding compounds of Formula II′ can be prepared by the same method outlined in Scheme 2 using the appropriate 4-chloro-thieno[2,3-d]pyrimidine or pyridine.

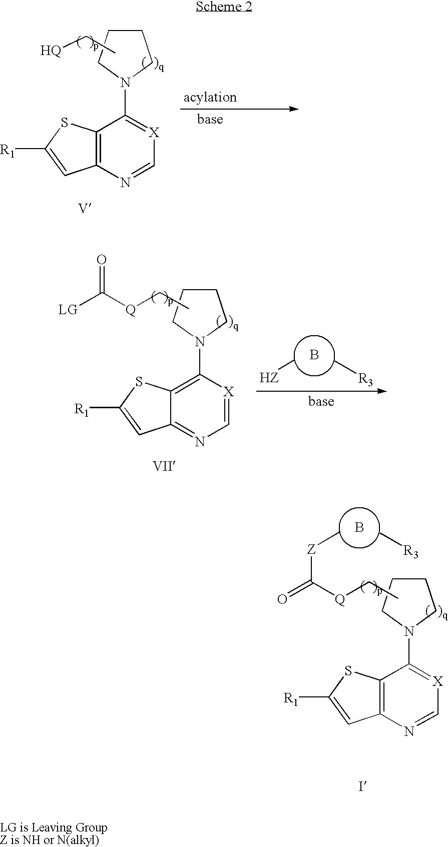

An alternative method to prepare FLT3 inhibitor compounds of Formula I′, wherein Q is 0, Z is NH, and p, q, B, X, R₁ and R₃ are as defined in Formula I′, is illustrated in Scheme 3. Treatment of alcohol intermediate V′, prepared as described in Scheme 1, with an appropriate isocyanate in the presence of a base such as triethylamine can provide the final product I′. The isocyanates are either commercially available or can be prepared by a known method (J. Org Chem, 1985, 50, 5879-5881). Corresponding compounds of Formula II′ can be prepared by the same method outlined in Scheme 3 using the appropriate 4-chloro-thieno[2,3-d]pyrimidine or pyridine.

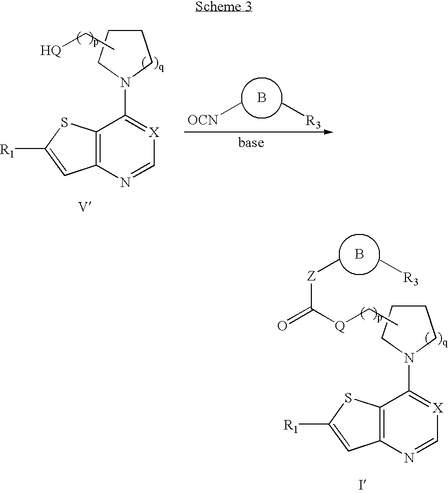

A method for preparing FLT3 inhibitor compounds of Formula I′, wherein Q is NH or N(alkyl), and p, q, B, X, Z, R₁ and R₃ are as defined in Formula I′, is outlined by the general synthetic route illustrated in Scheme 4. Treatment of the appropriate 4-chloro-thieno[3,2-d]pyrimidine or pyridine III′ with an N-protected aminocyclic amine VIII′, where PG is an amino protecting group known to those skilled in the art, in a solvent such as isopropanol at a temperature of 50° C. to 150° C. can provide intermediate IX′. Deprotection of the amino protecting group (PG) under standard conditions known in the art can provide compound X′. Acylation of X′ in the presence of a base such as diisopropylethylamine with an appropriate reagent VI′, wherein Z is NH or N(alkyl) and LG may be chloride, p-nitrophenoxy, or imidazole, or, when Z is CH₂, via coupling with an appropriate R₃BCH₂CO₂H using a standard coupling reagent such as 1-(3-dimethylaminopropyl)-3-ethylcarbodiimide hydrochloride (EDC) or 1-hydroxybenzotriazole (HOBT), can provide the final product I′. The amino cyclic amines are commercially available or are derived by known methods (U.S. Pat. No. 4,822,895; EP401623). The acylating reagents VI′ are either commercially available or can be prepared as outlined in Scheme 1. Additionally, FLT3 inhibitor compounds of Formula I′, wherein Z is NH, can be obtained by treatment of intermediate X′ with an appropriate isocyanate. The isocyanates are either commercially available or can be prepared by a known method (J. Org Chem, 1985, 50, 5879-5881). Corresponding compounds of Formula II′ can be prepared by the same method outlined in Scheme 4 using the appropriate 4-chloro-thieno[2,3-d]pyrimidine or pyridine.

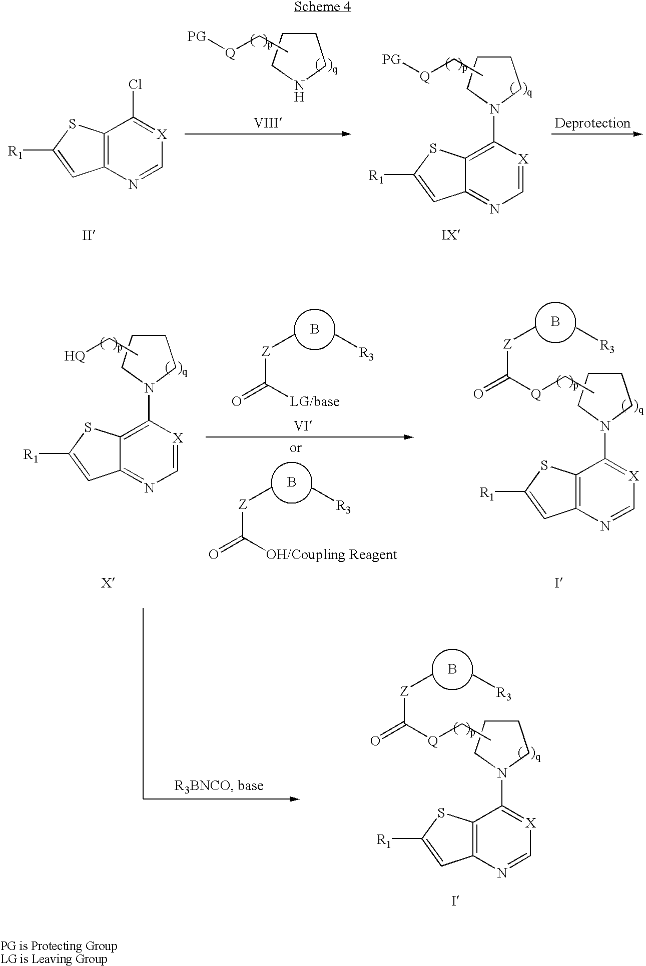

A method for preparing FLT3 inhibitor compounds of Formula I′, wherein Q is a direct bond, Z is NH or N(alkyl), and p, q, B, X, R₁ and R₃ are as defined in Formula I′, is outlined by the general synthetic route illustrated in Scheme 5 Reacting the appropriate 4-chloro-thieno[3,2-d]pyrimidine or pyridine III′ with a cyclic aminoester XI′ in a solvent such as isopropanol at a temperature of 50° C. to 150° C. followed by basic hydrolysis of the ester functionality can provide intermediate XII′. Coupling of an appropriate R₃BZH, wherein Z is NH or N(alkyl), to XII′ using a standard coupling reagent such as 1-(3-dimethylaminopropyl)-3-ethylcarbodiimide hydrochloride (EDC) or carbonyldiimidazole can provide final compound I′. Corresponding compounds of Formula II′ can be prepared by the same method outlined in Scheme 5 using the appropriate 4-chloro-thieno[2,3-d]pyrimidine or pyridine.

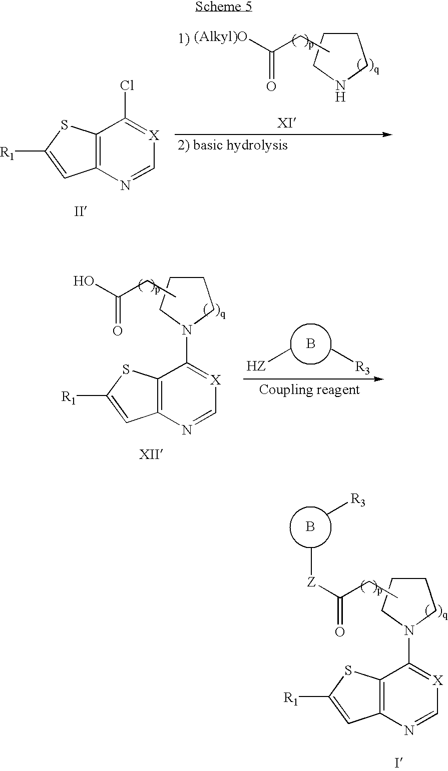

FLT3 inhibitor compounds of Formula I′ wherein R₁ is R_{bb}, and R_{bb} is aryl or heteroaryl, and Q, p, q, B, X, Z, and R₃ are as defined in Formula I′, can also be prepared as outlined in Scheme 6. Preparation of the appropriate bromothienopyrimidine/bromothienopyridine XIV′ can be derived from the known 6-bromo-4-chloro-thieno[3,2-d]pyrimidine or pyridine XIII′ (WO9924440) utilizing the reaction sequences outlined in schemes 1-5, in which XIII′ is used in place of II′. Treatment of bromide XIV′ with an appropriate aryl boronic acid or aryl boronic ester, wherein R is H or alkyl, in the presence of a palladium catalyst such as bis(triphenylphosphine)palladium dichloride in a solvent such as toluene at a temperature of 50° C. to 200° C. can provide the final product I′. The boronic acids/boronic esters are either commercially available or prepared by known methods (Synthesis 2003, 4, 469-483; Organic letters 2001, 3, 1435-1437). Corresponding compounds of Formula II′ can be prepared by the same method outlined in Scheme 6 using the appropriate 6-bromo-4-chloro-thieno[2,3-d]pyrimidine or pyridine.

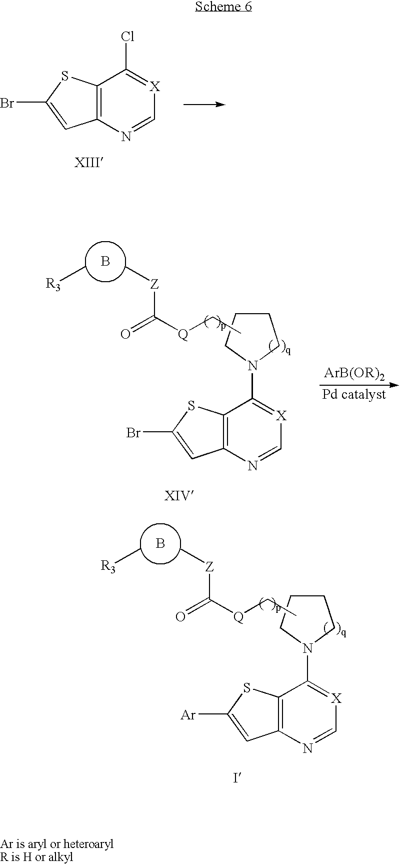

FLT3 inhibitor compounds of Formula I′, wherein R₁ is —CHCH(CH₂)ₙRₐ and Q, p, q, B, X, Z, and R₃ are as defined in Formula I′, can also be prepared as outlined in Scheme 7. Preparation of the appropriate bromothienopyrimidine/bromothienopyridine XIV′ can be derived from the known 6-bromo-4-chloro-thieno[3,2-d]pyrimidine or pyridine XIII′ (WO9924440) utilizing the reaction sequences outlined in schemes 1-5, in which XIII is used in place of II′. Treatment of XIV′ with an appropriate vinylstannane XV′ in the presence of a palladium catalyst such as bis(triphenylphosphine)palladium dichloride and a solvent such as dimethylformamide at a temperature of 25° C. to 150° C. can provide the alkenyl alcohol XVI′. Conversion of the alcohol XVI′ to an appropriate leaving group known by those skilled in the art such as a mesylate, followed by an SN₂ displacement reaction of XVII′ with an appropriate nucleophilic heterocycle, heteroaryl, amine, alcohol, sulfonamide, or thiol can provide the final compound I′. The corresponding cis olefin isomers of Formula I′ can be prepared by the same method utilizing the appropriate cis vinyl stannane. If Rₐ nucleophile is a thiol, further oxidation of the thiol can provide the corresponding sulfoxides and sulfones. If Rₐ nucleophile is an amino, acylation of the nitrogen with an appropriate acylating or sulfonylating agent can provide the corresponding amides, carbamates, ureas, and sulfonamides. If the desired Rₐ is COOR_{y} or CONR_{w}Rₓ, these can be derived from the corresponding hydroxyl group. Oxidation of the hydroxyl group to the acid followed by ester or amide formation under conditions known in the art can provide examples wherein Rₐ is COOR_{y} or CONR_{w}Rₓ. FLT3 inhibitor compounds of Formula I′ that have R₁ as a (CH₂)ₙRₐ can be derived from the corresponding alkene I′ by reduction of the olefin under conditions known in the art. Corresponding compounds of Formula II′ can be prepared by the same method outlined in Scheme 7 using the appropriate 6-bromo-4-chloro-thieno[2,3-d]pyrimidine or pyridine.

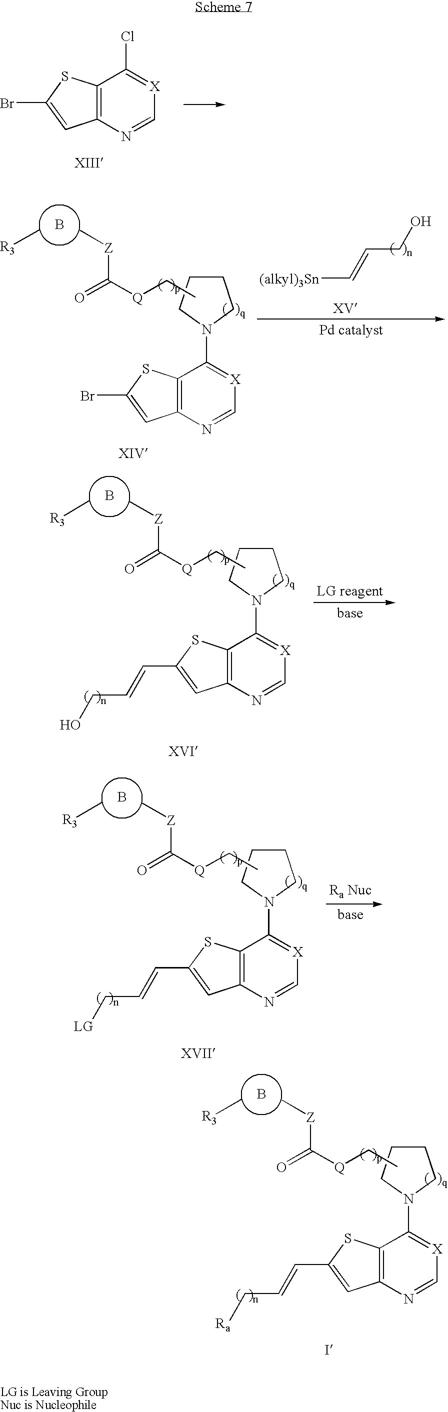

FLT3 inhibitor compounds of Formula I′, wherein R₁ is —CC(CH₂)ₙRₐ and Q, p, q, B, X, Z, and R₃ are as defined in Formula I′, can also be prepared as outlined in Scheme 8. Preparation of the appropriate bromothienopyrimidine/bromothienopyridine XIV′ can be derived from the known 6-bromo-4-chloro-thieno[3,2-d]pyrimidine or pyridine XIII′ (WO9924440) utilizing the reaction sequences outlined in schemes 1-5, in which XIII′ is used in place of II′. Treatment of XIV′ with an appropriate alkynyl alcohol in the presence of a palladium catalyst such as bis(triphenylphosphine)palladium dichloride, a copper catalyst such as copper(I)iodide, a base such as diethylamine and a solvent such as dimethylformamide at a temperature of 25° C. to 150° C. can provide the alkynyl alcohol XVIII′. Conversion of the alcohol XVIII′ to an appropriate leaving group known by those skilled in the art such as a mesylate, followed by an SN₂ displacement reaction of XIX′ with an appropriate nucleophilic heterocycle, heteroaryl, amine, alcohol, sulfonamide, or thiol can provide the final compound I′. If Rₐ nucleophile is a thiol, further oxidation of the thiol can provide the corresponding sulfoxides and sulfones. If Rₐ nucleophile is an amino, acylation of the nitrogen with an appropriate acylating or sulfonylating agent can provide the corresponding amides, carbamates, ureas, and sulfonamides. If the desired Rₐ is COOR_{y} or CONR_{w}Rₓ, these can be derived from the corresponding hydroxyl group. Oxidation of the hydroxyl group to the acid followed by ester or amide formation under conditions known in the art can provide examples wherein Rₐ is COOR_{y} or CONR_{w}Rₓ. Corresponding compounds of Formula II′ can be prepared by the same method outlined in Scheme 8 using the appropriate 6-bromo-4-chloro-thieno[2,3-d]pyrimidine or pyridine. 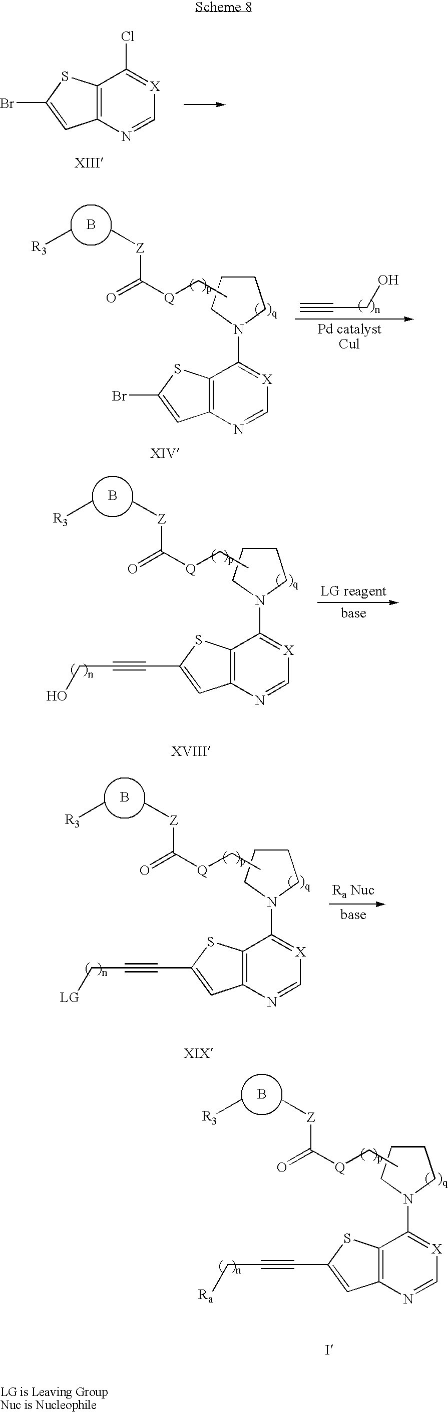
Representative FLT3 Inhibitors of Formula I′ and Formula II′

Representative FLT3 inhibitors of Formula I′ and Formula II′ synthesized by the afore-mentioned methods are presented hereafter. Examples of the synthesis of specific compounds are presented thereafter. Preferred compounds are numbers 5, 9, 11, 15, 18, 19, 25 and 26; particularly preferred are numbers 5, 9, 11, 25, and 26.

| Number | Compound |
|---|---|
| 1 | 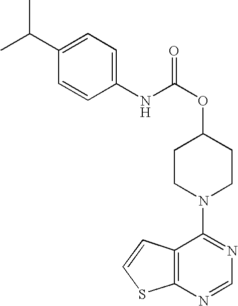 |
| 2 | 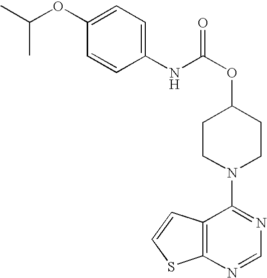 |
| 3 | 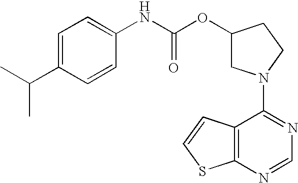 |
| 4 | 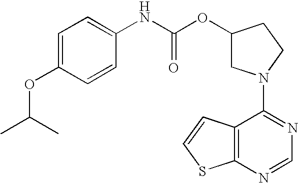 |
| 5 | 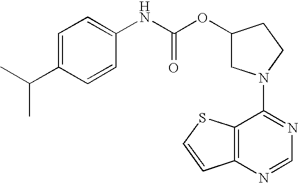 |
| 6 | 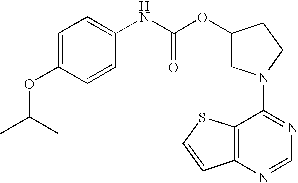 |
| 7 | 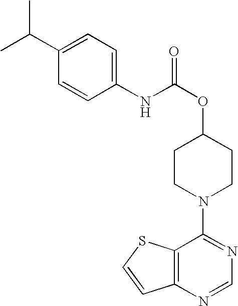 |
| 8 | 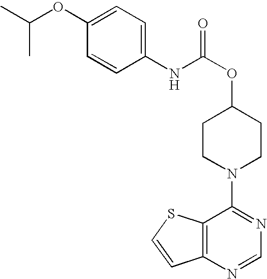 |
| 9 | 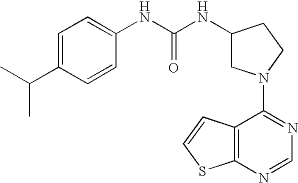 |
| 10 | 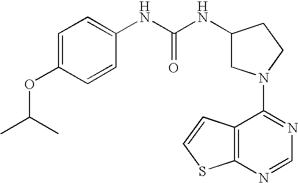 |
| 11 | 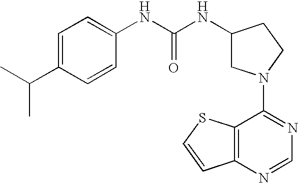 |
| 12 | 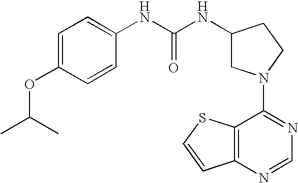 |
| 13 | 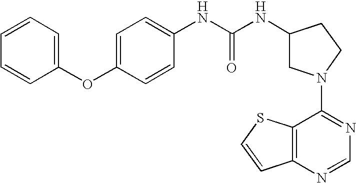 |
| 14 | 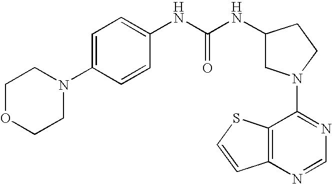 |
| 15 | 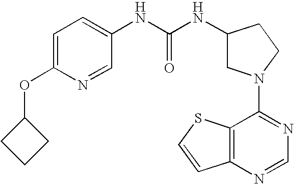 |
| 16 | 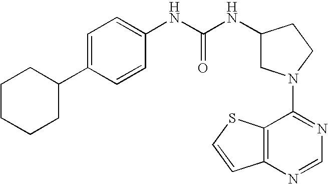 |
| 17 | 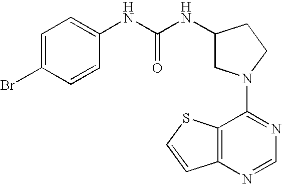 |
| 18 | 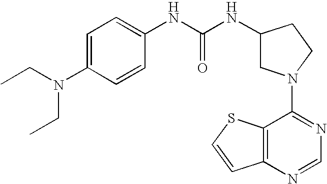 |
| 19 | 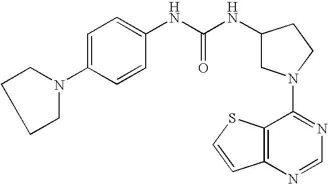 |
| 20 | 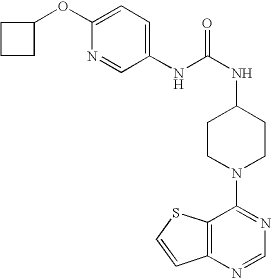 |
| 21 | 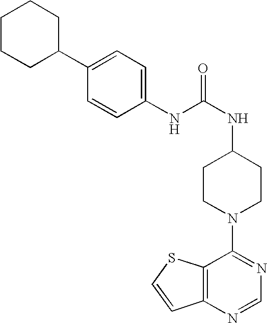 |
| 22 | 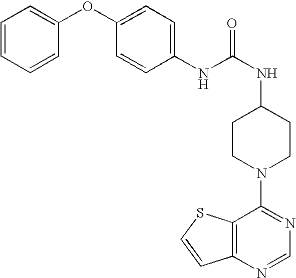 |
| 23 | 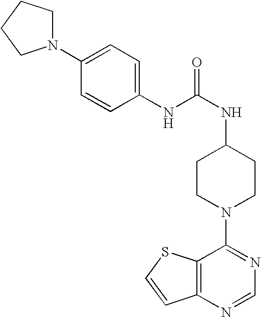 |
| 24 | 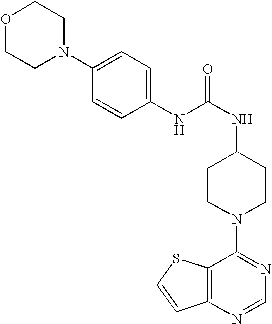 |
| 25 | 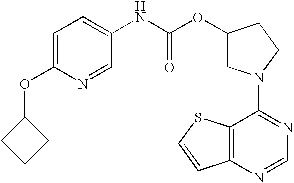 |
| 26 | 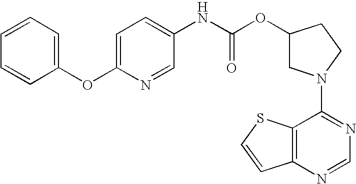 |
| 27 | 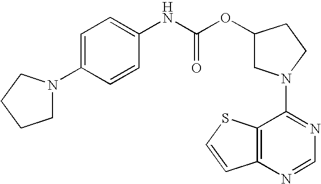 |
| 28 | 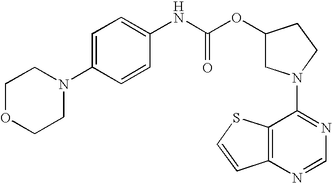 |
| 29 | 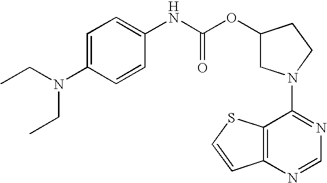 |

### EXAMPLE 1

### (4-Isopropyl-phenyl)-carbamic acid 1-thieno[2,3-d]pyrimidin-4-yl-piperidin-4-yl ester

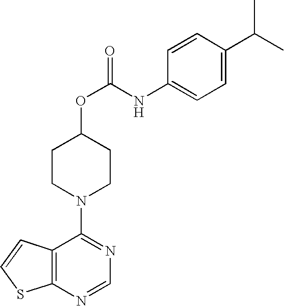

### a. 1-Thieno[2,3-d]pyrimidin-4-yl-piperidin-4-ol

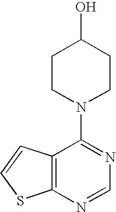

A solution of 4-chloro-thieno[2,3-d]pyrimidine (85.3 mg, 0.502 mmol) in isopropanol (2 mL) was treated with 4-hydroxypiperidine (50.6 mg, 0.501 mmol). After stirring at 100° C., overnight, the reaction was cooled to RT, partitioned between DCM (20 mL) and H₂O (20 mL). The organic phase was dried over Na₂SO₄ and concentrated in vacuo to afford the title compound as a solid (67.8 mg, 58%), which was used in the next step without further purification or characterization.

### b. (4-Isopropyl-phenyl)-carbamic acid 1-thieno[2,3-d]pyrimidin-4-yl-piperidin-4-yl ester

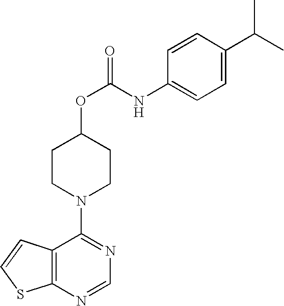

To a solution of 1,1′-carbonyldiimidazole (23.5 mg, 0.145 mmol) in DCM (1 mL) was added 4-isopropylaniline (19.6 mg, 0.145 mmol). After stirring at 0° C. for 2 h, 1-thieno[2,3-d]pyrimidin-4-yl-piperidin-4-ol (34.1 mg, 0.145 mmol), as prepared in the previous step, was added and stirred at RT. After 2 h, DMAP (17.7 mg, 0.145 mmol) was added and stirred at 85° C. overnight. The reaction was then cooled to RT, partitioned between DCM (10 mL) and H₂O (10 mL). The organic phase was dried over Na₂SO₄ and concentrated in vacuo. Purification by prep tlc (1:1 Hexane/EtOAc) afforded the title compound as a light brown solid (9.8 mg, 17%). ¹H NMR (300 MHz, CDCl₃) δ 8.7 (br s, 1H), 7.46 (br m, 1H), 7.30 (m, 3H), 7.17 (m, 2H), 6.65 (br s, 1H), 5.10 (m, 1H), 4.18 (m, 2H), 3.75 (m, 2H), 2.88 (heptet, 1H), 2.12 (m, 2H), 1.87 (m, 2H), 1.23 (d, 6H). LC/MS (ESI): calcd mass 396.2, found 397.2 [M+1]⁺.

### EXAMPLE 2

### (4-Isopropoxy-phenyl)-carbamic acid 1-thieno[2,3-d]pyrimidin-4-yl-piperidin-4-yl ester

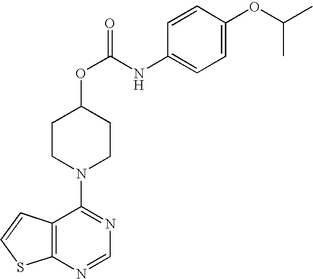

To a solution of 1,1′-carbonyldiimidazole (23.3 mg, 0.144 mmol) in DCM (1 mL) was added 4-isopropoxyaniline (21.7 mg, 0.144 mmol). After stirring at 0° C. for 2 h, 1-thieno[2,3-d]pyrimidin-4-yl-piperidin-4-ol (33.7 mg, 0.143 mmol), as prepared in Example 1a, was added and stirred at RT. After 2 h, DMAP (17.6 mg, 0.144 mmol) was added and stirred at 85° C. overnight. The reaction was then cooled to RT, partitioned between DCM (10 mL) and H₂O (10 mL). The organic phase was dried over Na₂SO₄ and concentrated in vacuo. Purification by prep tlc (1:1 Hexane/EtOAc) afforded the title compound as a light green solid (8.4 mg, 14%). ¹H NMR (300 MHz, CDCl₃) δ 8.7 (br s, 1H), 7.44 (br m, 1H), 7.29 (m, 3H), 6.85 (m, 2H), 6.56 (br s, 1H), 5.09 (m, 1H), 4.48 (heptet, 1H), 4.17 (m, 2H), 3.75 (m, 2H), 2.11 (m, 2H), 1.87 (m, 2H), 1.31 (d, 6H). LC/MS (ESI): calcd mass 412.2, found 413.2 [M+1]⁺.

### EXAMPLE 3

### (4-Isopropyl-phenyl)-carbamic acid 1-thieno[2,3-d]pyrimidin-4-yl-pyrrolidin-3-yl ester

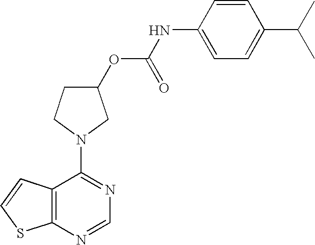

### a. (4-Isopropyl-phenyl)-carbamic acid 4-nitro-phenyl ester

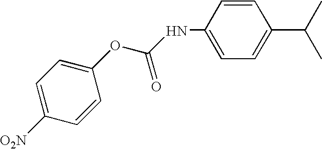

To a solution of 4-isopropylaniline (3.02 g, 22.3 mmol) in DCM (40 mL) and pyridine (10 mL) was added 4-nitrophenyl chloroformate (4.09 g, 20.3 mmol) portionwise with stirring over ˜30 sec with brief ice-bath cooling. After stirring at rt for 1 h, the homogeneous solution was diluted with DCM (100 mL) and washed with 0.6 M HCl (1×250 mL), 0.025 M HCl (1×400 mL), water (1×100 mL), and 1 M NaHCO₃ (1×100 mL). The organic layer was dried (Na₂SO₄) and concentrated to give the title compound as a light peach-colored solid (5.80 g, 95%). ¹H NMR (300 MHz, CDCl₃) δ 8.28 (m, 2H), 7.42-7.32 (m, 4H), 7.23 (m, 2H), 6.93 (br s, 1H), 2.90 (h, J=6.9 Hz, 1H), 1.24 (d, J=6.9 Hz, 6H). LC/MS (ESI): calcd mass 300.1, found 601.3 (2MH)⁺.

### b. (4-Isopropyl-phenyl)-carbamic acid 1-thieno[2,3-d]pyrimidin-4-yl-pyrrolidin-3-yl ester

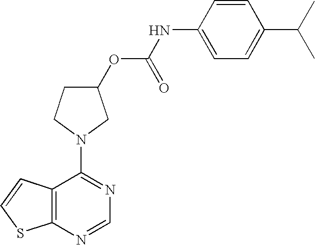

A mixture of pyrrolidin-3-ol (15.3 mg, 176 μmol), 4-chloro-thieno[2,3-d]pyrimidine (30.3 mg, 178 μmol) (Maybridge), DIEA (32 μL, 194 μmol), and DMSO-d₆ (117 μL) was stirred at 80° C. for 1 h. The reaction was then allowed to cool to rt, (4-isopropyl-phenyl)-carbamic acid 4-nitro-phenyl ester (68.1 mg, 227 μmol), as prepared in the previous step, was added, followed by NaH (dry) (5.4 mg, 225 μmol). The mixture was stirred (loosely capped) at rt for 5 min until the majority of gas evolution had subsided, and was then stirred at 80° C. for 20 min. The reaction was allowed to cool to rt, shaken with 2.0 M K₂CO₃ (1×2 mL), and extracted with DCM (2×2 mL), with phases separated by centrifugal force. The organic layers were combined, dried (Na₂SO₄), and concentrated. Flash chromatography of the residue (3:1 EtOAc/hex) provided the title compound as an off-white powder (49.1 mg, 72%). ¹H NMR (300 MHz, CDCl₃) δ 8.47 (s, 1H), 7.46 (d, 1H), 7.28 (m, 2H), 7.22 (d, 1H), 7.16 (m, 2H), 6.67 (br s, 1H), 5.53 (m, 1H), 4.12-3.90 (m, 4H), 2.86 (heptet, 1H), 2.43-2.22 (m, 2H), 1.22 (d, 6H). LC/MS (ESI): calcd mass 382.2, found 383.2 (MH)⁺.

### EXAMPLE 4

### (4-Isopropoxy-phenyl)-carbamic acid 1-thieno[2,3-d]pyrimidin-4-yl-pyrrolidin-3-yl ester

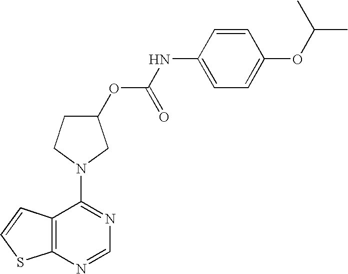

### a. (4-Isopropoxy-phenyl)-carbamic acid 4-nitro-phenyl ester

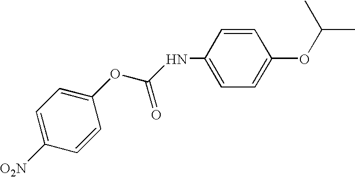

Prepared essentially as described for Example 3a using 4-isopropoxyaniline, except the water and 1M NaHCO₃ washes were omitted. The title compound was obtained as a light violet-white solid (16.64 g, 98%). ¹H NMR (300 MHz, CDCl₃) δ 8.26 (m, 2H), 7.40-7.28 (m, 4H), 6.98 (br s, 1H), 6.87 (m, 2H), 4.50 (heptet, J=6.0 Hz, 1H), 1.33 (d, J=6.0 Hz, 6H). LC/MS (ESI): calcd mass 316.1, found 633.2 (2 MH)⁻.

### b. (4-Isopropoxy-phenyl)-carbamic acid 1-thieno[2,3-d]pyrimidin-4-yl-pyrrolidin-3-yl ester

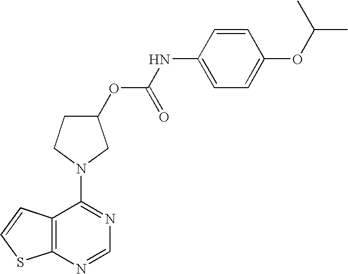

Prepared essentially as described for Example 3b using 4-chloro-thieno[2,3-d]pyrimidine (Maybridge) and (4-isopropoxy-phenyl)-carbamic acid 4-nitro-phenyl ester (prepared in the previous step), except the S_{N}Ar reaction was performed at 80° C. for 1 h, and 1.6 eq NaH was used. Flash chromatography (3:1 EtOAc/hex) provided the title compound as an off-white powder (44.3 mg, 73%). ¹H NMR (300 MHz, CDCl₃) δ 8.47 (s, 1H), 7.46 (d, J=6.1 Hz, 1H), 7.28-7.21 (m, 3H), 6.83 (m, 2H), 6.55 (br s, 1H), 5.52 (m, 1H), 4.47 (heptet, J=6.1 Hz, 1H), 4.14-3.90 (m, 4H), 2.43-2.20 (m, 2H), 1.31 (d, J=6.1 Hz, 6H). LC/MS (ESI): calcd mass 398.1, found 399.2 (MH)⁺.

### EXAMPLE 5

### (4-Isopropyl-phenyl)-carbamic acid 1-thieno[3,2-d]pyrimidin-4-yl-pyrrolidin-3-yl ester

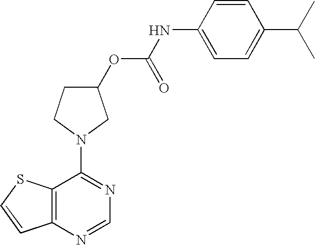

Prepared essentially as described for Example 3b using 4-chloro-thieno[3,2-d]pyrimidine (Maybridge) and (4-isopropyl-phenyl)-carbamic acid 4-nitro-phenyl ester (prepared in Example 3a), except the S_{N}Ar reaction was performed at 80° C. for 1 h. Flash chromatography (3:4 hex/acetone) provided the title compound (38.5 mg, 59%). ¹H NMR (300 MHz, CDCl₃) δ 8.53 (s, 1H), 7.75 (d, 1H), 7.42 (d, 1H), 7.28 (m, 2H), 7.16 (m, 2H), 6.74 (br s, 1H), 5.53 (m, 1H), 4.21-3.92 (m, 4H), 2.87 (heptet, 1H), 2.43-2.22 (m, 2H), 1.22 (d, 6H). LC/MS (ESI): calcd mass 382.2, found 383.2 (MH)⁺.

### EXAMPLE 6

### (4-Isopropoxy-phenyl)-carbamic acid 1-thieno[3,2-d]pyrimidin-4-yl-pyrrolidin-3-yl ester

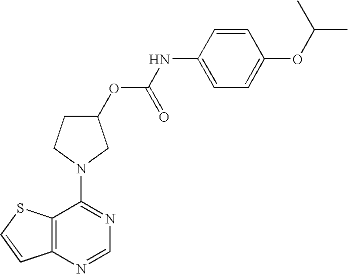

Prepared essentially as described for Example 3b using 4-chloro-thieno[3,2-d]pyrimidine (Maybridge) and (4-isopropoxy-phenyl)-carbamic acid 4-nitro-phenyl ester (prepared in Example 4a), except the S_{N}Ar reaction was performed at 80° C. for 1 h. Flash chromatography (3:4 hex/acetone) provided the title compound (43.1 mg, 69%). ¹H NMR (300 MHz, CDCl₃) δ 8.54 (s, 1H), 7.76 (d, 1H), 7.43 (d, 1H), 7.25 (m, 2H), 6.83 (m, 2H), 6.60 (br s, 1H), 5.52 (m, 1H), 4.48 (heptet, 1H), 4.22-3.92 (m, 4H), 2.43-2.22 (m, 2H), 1.31 (d, 6H). LC/MS (ESI): calcd mass 398.1, found 399.2 (MH)⁺.

### EXAMPLE 7

### (4-Isopropyl-phenyl)-carbamic acid 1-thieno[3,2-d]pyrimidin-4-yl-piperidin-4-yl ester

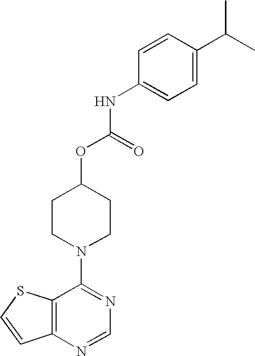

Prepared essentially as described for Example 3b using 4-chloro-thieno[3,2-d]pyrimidine (Maybridge), 4-hydroxypiperidine (Acros, less than 1% water, K.F.), and (4-isopropyl-phenyl)-carbamic acid 4-nitro-phenyl ester (prepared in Example 3a), except 1.4 eq NaH was used. Flash chromatography (1:4 hex/EtOAc) provided the title compound (23.7 mg, 31%). ¹H NMR (300 MHz, CDCl₃) δ 8.60 (s, 1H), 7.75 (d, 1H), 7.46 (d, 1H), 7.35-7.25 (m, 2H), 7.18 (m, 2H), 6.60 (br s, 1H), 5.10 (m, 1H), 4.36-4.25 (m, 2H), 3.92-3.80 (m, 2H), 2.88 (heptet, 1H), 2.20-2.07 (m, 2H), 1.93-1.80 (m, 2H), 1.23 (d, 6H). LC/MS (ESI): calcd mass 396.2, found 397.2 (MH)⁺.

### EXAMPLE 8

### (4-Isopropoxy-phenyl)-carbamic acid 1-thieno[3,2-d]pyrimidin-4-yl-piperidin-4-yl ester

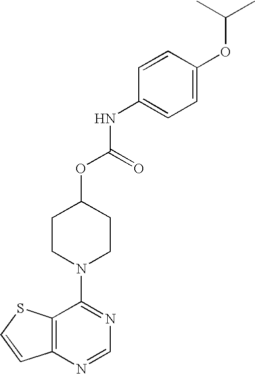

Prepared essentially as described for Example 3b using 4-chloro-thieno[3,2-d]pyrimidine (Maybridge), 4-hydroxypiperidine (Acros, less than 1% water, K.F.), and (4-isopropoxy-phenyl)-carbamic acid 4-nitro-phenyl ester (prepared in Example 4a), except 1.7 eq NaH was used. Flash chromatography (1:4 hex/EtOAc) provided the title compound (42.1 mg, 62%). ¹H NMR (300 MHz, CDCl₃) δ 8.60 (s, 1H), 7.74 (d, 1H), 7.44 (d, 1H), 7.29 (m, 2H), 6.85 (m, 2H), 6.59 (br s, 1H), 5.09 (m, 1H), 4.49 (heptet, 1H), 4.35-4.21 (br m, 2H), 3.91-3.79 (m, 2H), 2.17-2.05 (m, 2H), 1.92-1.78 (m, 2H), 1.32 (d, 6H). LC/MS (ESI): calcd mass 412.2, found 413.2 (MH)⁺.

### EXAMPLE 9

### 1-(4-Isopropyl-phenyl)-3-(1-thieno[2,3-d]pyrimidin-4-yl-pyrrolidin-3-yl)-urea

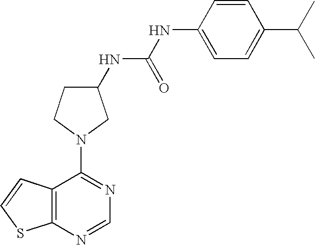

To a mixture of 4-chloro-thieno[2,3-d]pyrimidine (Maybridge) (25.4 mg, 149 μmol), 3-(tert-butoxycarbonylamino)pyrrolidine (TCI America) (27.1 mg, 146 μmol), and DIEA (27.5 μL, 166 μmol) was added DMSO (100 μL), and the reaction was stirred at 100° C. for 20 min. The reaction solution was allowed to cool to rt, TFA (230 μL, 2.98 mmol) was added in one portion, and the reaction stirred at 100° C. for 5 min. After cooling to rt, the reaction was partitioned with DCM (2 mL) and 2.5 M NaOH (2 mL), and the organic layer was collected and concentrated without drying to give the intermediate pyrrolidinylamine which was used immediately for the next step without further purification or characterization. To this intermediate was added (4-isopropyl-phenyl)-carbamic acid 4-nitro-phenyl ester (58.8 mg, 196 μmol), prepared as described in Example 3a, and CH₃CN (100 μL), and the reaction was heated at 100° C. for 15 min. After cooling to rt, the reaction was partitioned with DCM (2 mL) and 2 M K₂CO₃ (2 mL), the aqueous layer was extracted with DCM (1×2 mL), and the organic layers were combined, dried (Na₂SO₄), and concentrated. Purification with silica flash chromatography (1:1 hex/acetone) afforded the title compound (26.3 mg, 47%). ¹H NMR (300 MHz, CDCl₃) δ 8.28 (s, 1H), 7.26-7.21 (m, 3H), 7.13 (m, 2H), 7.09 (d, 1H), 7.00 (br s, 1H), 6.25 (br d, 1H), 4.60 (m, 1H), 3.96-3.79 (m, 4H), 2.84 (heptet, 1H), 2.30-2.14 (m, 2H), 1.20 (d, 6H). LC/MS (ESI): calcd mass 381.2, found 382.2 (MH)⁺.

### EXAMPLE 10

### 1-(4-Isopropoxy-phenyl)-3-(1-thieno[2,3-d]pyrimidin-4-yl-pyrrolidin-3-yl)-urea

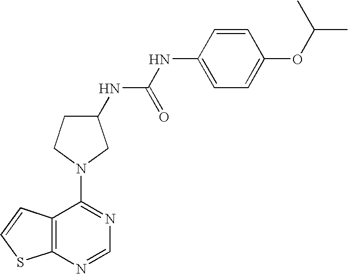

Prepared essentially as described for Example 9, using (4-isopropoxy-phenyl)-carbamic acid 4-nitro-phenyl ester as prepared in Example 4a. Flash chromatography (1:1 hex/acetone) afforded the title compound (25.8 mg, 45%). ¹H NMR (300 MHz, CDCl₃) δ 8.31 (s, 1H), 7.29 (d, 1H), 7.19 (m, 2H), 7.11 (d, 1H), 6.81 (m, 2H), 6.75 (br s, 1H), 5.90 (br d, 1H), 4.59 (m, 1H), 4.46 (heptet, 1H), 4.02-3.90 (m, 1H), 3.89-3.76 (m, 3H), 2.32-2.15 (m, 2H), 1.30 (d, 6H). LC/MS (ESI): calcd mass 397.2, found 398.2 (MH)³⁰ .

### EXAMPLE 11

### 1-(4-Isopropyl-phenyl)-3-(1-thieno[3,2-d]pyrimidin-4-yl-pyrrolidin-3-yl)-urea

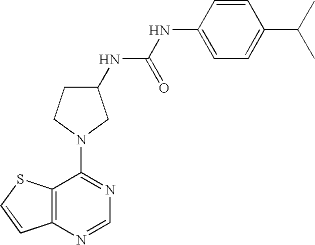

Prepared essentially as described for Example 9, using 4-chloro-thieno[3,2-d]pyrimidine (Maybridge). Flash chromatography (1:2 hex/acetone) afforded the title compound (17.2 mg, 30%). ¹H NMR (300 MHz, CDCl₃) δ 8.32 (s, 1H), 7.71 (d, 1H), 7.36 (br s, 1H), 7.34 (d, 1H), 7.27 (m, 2H), 7.12 (m, 2H), 6.76 (br d, 1H), 4.62 (m, 1H), 3.87-3.66 (m, 4H), 2.84 (heptet, 1H), 2.32-2.15 (m, 2H), 1.20 (d, 6H). LC/MS (ESI): calcd mass 381.2, found 382.2 (MH)⁺.

### EXAMPLE 12

### 1-(4-Isopropoxy-phenyl)-3-(1-thieno[3,2-d]pyrimidin-4-yl-pyrrolidin-3-yl)-urea

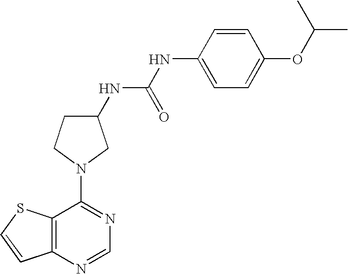

Prepared essentially as described for Example 9, using 4-chloro-thieno[3,2-d]pyrimidine (Maybridge) and (4-isopropoxy-phenyl)-carbamic acid 4-nitro-phenyl ester as prepared in Example 4a. Flash chromatography (1:2→1:3 hex/acetone) afforded the title compound (23.3 mg, 39%). ¹H NMR (300 MHz, CDCl₃) δ 8.34 (s, 1H), 7.71 (d, 1H), 7.34 (d, 1H), 7.22 (m, 2H), 7.14 (br s, 1H), 6.81 (m, 2H), 6.48 (br d, 1H), 4.60 (m, 1H), 4.45 (heptet, 1H), 3.94-3.74 (m, 4H), 2.27-2.16 (m, 2H), 1.30 (d, 6H). LC/MS (ESI): calcd mass 397.2, found 398.2 (MH)⁺.

### EXAMPLE 13

### 1-(4-Phenoxy-phenyl)-3-(1-thieno[3,2-d]pyrimidin-4-yl-pyrrolidin-3-yl)-urea

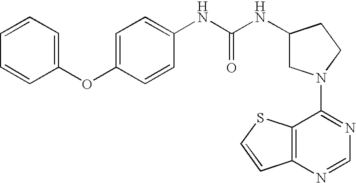

### a. (1-Thieno[3,2-d]pyrimidin-4-yl-pyrrolidin-3-yl)-carbamic acid tert-butyl ester

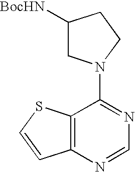

A solution of 4-chlorothieno[3,2-d]pyimidine (400 mg, 2.35 mmol), Pyrrolidine-3-yl-carbamic acid tert-butyl ester (436 mg, 2.35 mmol), diisopropylethylamine (285 mg, 2.82 mmol) in isopropanol (10 mL) was heated to 100° C. for 1 hr. The resulting mixture was cooled to RT, poured into ethyl acetate (50 mL), and washed with water (25 mL). The organic layer was dried over anhydrous sodium sulfate, concentrated, and purified by silica gel chromatography (5% MeOH/EtOAc) to provide the title compound (645 mg, 86% yield). ¹H NMR (400 MHz, CD₃OD) δ 8.34 (s, 1H), 8.02 (d, 1H), 7.32 (d, 1H), 4.23 (m, 1H), 4.18-3.92 (m, 3H), 3.78 (m, 1H), 2.26 (m, 1H), 2.04 (m, 1H), 1.42 (s, 9H). LC/MS (ESI): calcd mass 320.1, found 321.2 (MH)⁺.

### b. 1-Thieno[3,2-d]pyrimidin-4-yl-pyrrolidin-3-ylamine hydrochloride

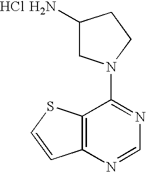

A solution of (1-Thieno[3,2-d]pyrimidin-4-yl-pyrrolidin-3-yl)-carbamic acid tert-butyl ester (645 mg, 2.02 mmol), 2 M HCl/Et₂O (4 mL), and CH₂Cl₂ (20 mL) was stirred at RT for 16 h. The resulting solid was filtered and washed with EtOAc to provide the title compound as an off-white solid (491 mg, 95%). LC/MS (ESI): calcd mass 220.1, found 221.1 (MH)⁺.

### c. 1-(4-Phenoxy-phenyl)-3-(1-thieno[3,2-d]pyrimidin-4-yl-pyrrolidin-3-yl)-urea

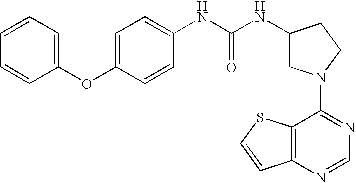

To a solution of 1-Thieno[3,2-d]pyrimidin-4-yl-pyrrolidin-3-ylamine hydrochloride (21 mg, 0.082 mmol) and diisopropylethylamine (17.3 mg, 0.172 mmol) in CH₂Cl₂ (0.5 mL) was added 4-phenoxyphenyl isocyanate (21 mg, 0.99 mmol). The resulting solution was stirred at RT for 16 h, then poured into 1 M HCl (5 mL) and extracted with CH₂Cl₂ (10 mL). The organic layer was dried over anhydrous sodium sulfate, concentrated, and purified by silica gel chromatography (2% MeOH/CH₂Cl₂) to provide the title compound (17 mg) as a white solid. ¹H NMR (400 MHz, CD₃OD) δ 8.36 (s, 1H), 8.05 (d, 1H), 7.35-7.28 (m, 5H), 7.05 (m, 1H), 6.92 (m, 4H), 4.49 (m, 1H), 4.22-3.98 (m, 3H), 3.87 (m, 1H), 2.36 (m, 1H), 2.11 (m, 1H). LC/MS (ESI): calcd mass 431.1, found 432.1 (MH)⁺.

### EXAMPLE 14

### 1-(4-Morpholin-4-yl-phenyl)-3-(1-thieno[3,2-d]pyrimidin-4-yl-pyrrolidin-3-yl)-urea

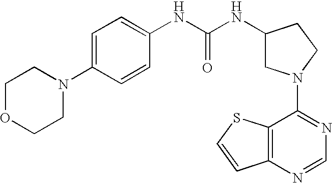

### a. (4-Morpholin-4-yl-phenyl)-carbamic acid 4-nitro-phenyl ester; hydrochloride

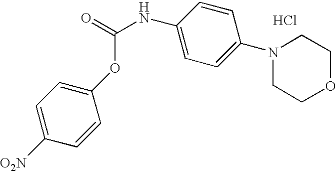

A solution of 4-nitrophenyl chloroformate (798 mg, 3.96 mmol) in THF (2.0 mL) was added rapidly by syringe over 10 s at rt under air to a stirred solution of 4-morpholin-4-yl-phenylamine (675 mg, 3.79 mmol) in THF (8.8 mL), with a heavy grey precipitate forming “instantly”. The reaction was immediately capped and stirred “rt” for ˜30 min (vial spontaneously warmed), and was then filtered. The grey filter cake was washed with dry THF (2×10 mL), and dried under high vacuum at 80° C. to afford the title compound as a grey powder (1.361 g, 95%). A portion was partitioned with CDCl₃ and aqueous 0.5 M trisodium citrate to generate the CDCl₃-soluble free base: ¹H-NMR (300 MHz, CDCl₃) δ 8.28 (m, 2H), 7.42-7.31 (m, 4H), 6.95-6.88 (m, 3H), 3.87 (m, 4H), 3.14 (m, 4H).

### b. 1-(4-Morpholin-4-yl-phenyl)-3-(1-thieno[3,2-d]pyrimidin-4-yl-pyrrolidin-3-yl)-urea

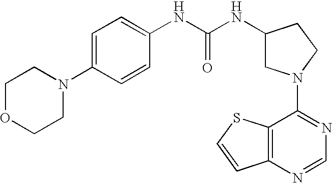

A solution of 1-Thieno[3,2-d]pyrimidin-4-yl-pyrrolidin-3-ylamine hydrochloride (15 mg, 0.059 mmol), prepared as described in Example 13b, diisopropylethylamine (12.4 mg, 0.123 mmol), (4-Morpholin-4-yl-phenyl)-carbamic acid 4-nitro-phenyl ester hydrochloride (22.2 mg, 0.059 mmol) and acetonitrile (0.5 mL) was heated at 90° C. for 2 h. The resulting solution was poured into CH₂Cl₂ (10 mL) and washed sequentially with 1 M NaOH (5 mL) and H₂O (5 mL). The organic layer was dried over anhydrous sodium sulfate, concentrated, and purified by silica gel chromatography (2% MeOH/CH₂Cl₂) to provide the title compound (15 mg) as a white solid. ¹H NMR (400 MHz, CD₃OD) δ 8.35 (s, 1H), 8.04 (d, 1H), 7.34 (d, 1H), 7.23 (m, 2H), 6.90 (m, 2H), 4.48 (m, 1H), 4.22-3.97 (m, 3H), 3.87-3.79 (m, 5H), 3.03 (m, 4H), 2.35 (m, 1H), 2.10 (m, 1H). LC/MS (ESI): calcd mass 424.2, found 425.1 (MH)⁺.

### EXAMPLE 15

### 1-(6-Cyclobutoxy-pyridin-3-yl)-3-(1-thieno[3,2-d]pyrimidin-4-yl-pyrrolidin-3-yl)-urea

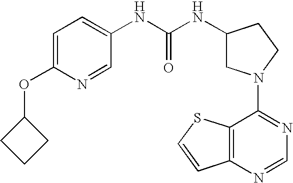

### a. 2-Cyclobutoxy-5-nitro-pyridine

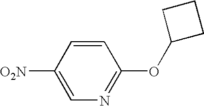

A mixture of 2-chloro-5-nitropyridine (7.12 g, 45.0 mmol) and cyclobutanol (3.40 g, 47.2 mmol) in THF (30 mL) was vigorously stirred at 0° C. while NaH (1.18 g, 46.7 mmol) was added in three portions over ˜10-20 s under air (Caution: Extensive gas evolution). Reaction residue was rinsed down with additional THF (5 mL), followed by stirring under positive argon pressure in the ice bath for 1-2 more minutes. The ice bath was then removed and the brown homogeneous solution was stirred at “rt” for 1 h. The reaction was concentrated under reduced pressure at 80° C., taken up in 0.75 M EDTA (tetrasodium salt) (150 mL), and extracted with DCM (1×100 mL, 1×50 mL). The combined organic layers were dried (Na₂SO₄), concentrated, taken up in MeOH (2×100 mL) and concentrated under reduced pressure at 60° C. to provide the title compound as a thick dark amber oil that crystallized upon standing (7.01 g, 80%). ¹H NMR (300 MHz, CDCl₃) δ 9.04 (dd, J=2.84 and 0.40 Hz, 1H), 8.33 (dd, J=9.11 and 2.85 Hz, 1H), 6.77 (dd, J=9.11 and 0.50 Hz, 1H), 5.28 (m, 1H), 2.48 (m, 2H), 2.17 (m, 2H), 1.87 (m, 1H), 1.72 (m, 1H).

### b. 6-Cyclobutoxy-pyridin-3-ylamine

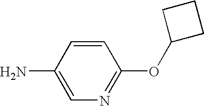

A flask containing 10% w/w Pd/C (485 mg) was gently flushed with argon while slowly adding MeOH (50 mL) along the sides of the flask, followed by the addition in ˜5 mL portions of a solution of 2-cyclobutoxy-5-nitro-pyridine (4.85 g, 25 mmol), as prepared in the previous step, in MeOH (30 mL). (Caution: Large scale addition of volatile organics to Pd/C in the presence of air can cause fire.) The flask was then evacuated one time and stirred under H2 balloon pressure for 2 h at rt. The reaction was then filtered, and the clear amber filtrate was concentrated, taken up in toluene (2×50 mL) to remove residual MeOH, and concentrated under reduced pressure to provide the crude title compound as a translucent dark brown oil with a faint toluene smell (4.41 g, “108%” crude yield). ¹H NMR (300 MHz, CDCl₃) δ 7.65 (d, J=3.0 Hz, 1H), 7.04 (dd, J=8.71 and 2.96 Hz, 1H), 6.55 (d, J=8.74 Hz, 1H), 5.04 (m, 1H), 2.42 (m, 2H), 2.10 (m, 2H), 1.80 (m, 1H), 1.66 (m, 1H). LC-MS (ESI): calcd mass 164.1, found 165.2 (MH⁺).

### c. (6-Cyclobutoxy-pyridin-3-yl)-carbamic acid 4-nitro-phenyl ester

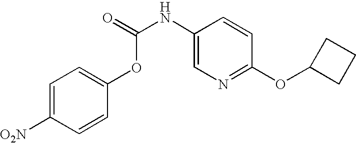

A mixture of 6-cyclobutoxy-pyridin-3-ylamine (4.41 g, assume 25 mmol), as prepared in the previous step, and CaCO₃ (3.25 g, 32.5 mmol) (10 micron powder) was treated with a homogeneous solution of 4-nitrophenyl chloroformate (5.54 g, 27.5 mmol) in toluene (28 mL) in one portion at rt, and was stirred at “rt” (reaction warmed spontaneously) for 2 h. The reaction mixture was then directly loaded onto a flash silica column (95:5 DCM/MeOH→9:1 DCM/MeOH) to afford 5.65 g of material, which was further purified by trituration with hot toluene (1×200 mL) to provide the title compound (4.45 g, 54%). ¹H NMR (400 MHz, CDCl₃) δ 8.28 (m, 2H), 8.12 (d, 1H), 7.81 (m, 1H), 7.39 (m, 2H), 6.85 (br s, 1H), 6.72 (d, 1H), 5.14 (m, 1H), 2.45 (m, 2H), 2.13 (m, 2H), 1.84 (m, 1H), 1.68 (m, 1H). LC-MS (ESI): calcd mass 329.1, found 330.1 (MH⁺).

### d. 1-(6-Cyclobutoxy-pyridin-3-yl)-3-(1-thieno[3,2-d]pyrimidin-4-yl-pyrrolidin-3-yl)-urea

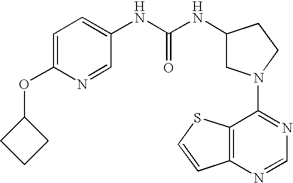

Prepared essentially as described in Example 14 using (6-Cyclobutoxy-pyridin-3-yl)-carbamic acid 4-nitro-phenyl ester in place of (4-morpholin-4-yl-phenyl)-carbamic acid 4-nitrophenyl ester hydrochloride. ¹H NMR (400 MHz, CD₃OD) δ 8.35 (s, 1H), 8.05 (m, 2H), 7.71 (dd, 1H), 7.33 (d, 1H), 6.68 (d, 1H), 5.02 (m, 1H), 4.47 (m, 1H), 4.22-3.97 (m, 3H), 3.85 (m, 1H), 2.47-2.31 (m, 3H), 2.08 (m, 3H), 1.82 (m, 1H), 1.69 (m, 1H). LC/MS (ESI): calcd mass 410.2, found 411.1 (MH)⁺.

### EXAMPLE 16

### 1-(4-Cyclohexyl-phenyl)-3-(1-thieno[3,2-d]pyrimidin-4-yl-pyrrolidin-3-yl)-urea

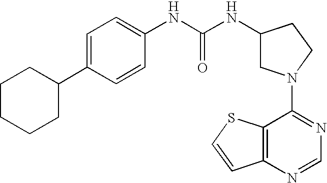

### a. (4-Cyclohexyl-phenyl)-carbamic acid 4-nitro-phenyl ester

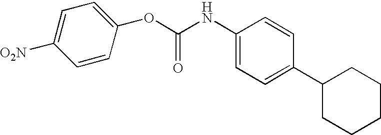

Prepared essentially as described in Example 3a except that 4-cyclohexylaniline was used in place of 4-isopropylaniline. ¹H NMR (DMSO-d₆) δ 10.37 (br, 1H), 8.30 (d, J=9.30 Hz, 2H), 7.52 (d, J=9.00 Hz, 2H), 7.41 (d, J=8.10 Hz, 2H), 7.18 (d, J=8.70 Hz, 2H), 1.18-1.82 (11H).

### b. 1-(4-Cyclohexyl-phenyl)-3-(1-thieno[3,2-d]pyrimidin-4-yl-pyrrolidin-3-yl)-urea

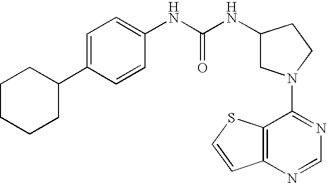

Prepared essentially as described in Example 14 using (4-Cyclohexyl-phenyl)-carbamic acid 4-nitro-phenyl ester in place of (4-morpholin-4-yl-phenyl)-carbamic acid 4-nitrophenyl ester hydrochloride. ¹H NMR (400 MHz, CD₃OD) δ 8.36 (s, 1H), 8.05 (d, 1H), 7.34 (d, 1H), 7.23 (m, 2H), 7.09 (m, 2H), 4.48 (m, 1H), 4.22-3.98 (m, 3H), 3.86 (m, 1H), 2.46-2.32 (m, 2H), 2.10 (m, 1H), 1.84-1.71 (m,5H), 1.47-1.22 (m, 5H). LC/MS (ESI): calcd mass 421.2, found 422.1 (MH)⁺.

### EXAMPLE 17

### 1-(4-Bromo-phenyl)-3-(1-thieno[3,2-d]pyrimidin-4-yl-pyrrolidin-3-yl)-urea

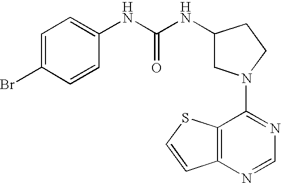

Prepared essentially as described in Example 13 using 4-bromophenyl isocyanate in place of 4-phenoxyphenyl isocyanate. ¹H NMR (400 MHz, CD₃OD) δ 8.36 (s, 1H), 8.05 (d, 1H), 7.38-7.29 (m, 5H), 4.48 (m, 1H), 4.22-3.98 (m, 3H), 3.87 (m, 1H), 2.37 (m, 1H), 2.12 (m, 1H). LC/MS (ESI): calcd mass 417.0, found 419.9 (MH)⁺.

### EXAMPLE 18

### 1-(4-Diethylamino-phenyl)-3-(1-thieno[3,2-d]pyrimidin-4-yl-pyrrolidin-3-yl)-urea

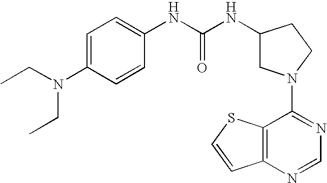

### a. (4-Diethylamino-phenyl)-carbamic acid 4-nitro-phenyl ester hydrochloride

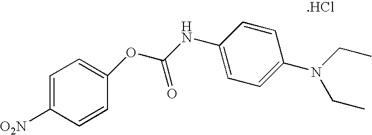

A solution of N,N-diethyl-benzene-1,4-diamine (2.21 g, 13.5 mmol) in DCM (30 mL) was added rapidly dropwise under air over two minutes to a stirred solution of 4-nitrophenyl chloroformate (2.86 g, 14.2 mmol) in DCM (7.4 mL) in an open beaker with rt water bath cooling. The resulting mixture was stirred at rt for 30 min, then filtered. The filter cake was powdered with mortar and pestle, shaken for one minute with DCM (20 mL), filtered, and the filter cake powdered as before to provide the title compound as an easily-handled beige powder (4.037 g, 82%). ¹H NMR (400 MHz, DMSO-d6) δ 12.77 (br s, 1H), 10.85 (br s, 1H), 8.33 (m, 2H), 7.81 (m, 2H), 7.72 (m, 2H), 7.57 (m, 2H), 3.52 (m, 4H), 1.04 (t, 6H).

### b. 1-(4-Diethylamino-phenyl)-3-(1-thieno[3,2-d]pyrimidin-4-yl-pyrrolidin-3-yl)-urea

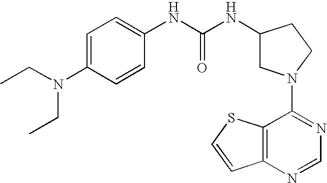

A solution of 1-thieno[3,2-d]pyrimidin-4-yl-pyrrolidin-3-ylamine hydrochloride (48 mg, 190 μmol), prepared as described in Example 13b, TEA (58 μL, 414 μmol), CHCl₃ (300 μL), and (4-diethylamino-phenyl)-carbamic acid 4-nitro-phenyl ester hydrochloride (77 mg, 210 μmol) were stirred at 80° C. for 20 min, then partitioned with DCM (2 mL) and 2.5M NaOH (2 mL). The aqueous layer was extracted with DCM (1×2 mL) and the organic layers were combined, dried (Na₂SO₄), and concentrated. Purification of the residue with C18 HPLC, followed by silica flash cartridge chromatography (EtOAc eluent) afforded the title compound (14.7 mg, 19%). ¹H NMR (400 MHz, CDCl₃) δ 8.46 (s, 1H), 7.72 (d, 1H), 7.38 (d, 1H), 7.05 (br m, 2H), 6.61 (br m, 2H), 6.19 (br s, 1H), 5.10 (br s, 1H), 4.61 (br s, 1H), 4.13 (m, 1H), 3.91 (m, 2H), 3.71 (m, 1H), 3.32 (br m, 4H), 2.31 (m, 1H), 2.03 (m, 1H), 1.13 (t, 6H). LC/MS (ESI): calcd mass 410.2, found 411.1 (MH)⁺.

### EXAMPLE 19

### 1-(4-Pyrrolidin-1-yl-phenyl)-3-(1-thieno[3,2-d]pyrimidin-4-yl-pyrrolidin-3-yl)-urea

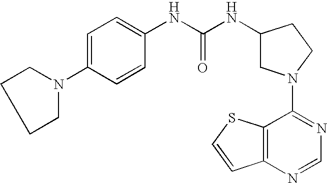

### a. (4-Pyrrolidin-1-yl-phenyl)-carbamic acid 4-nitro-phenyl ester hydrochloride

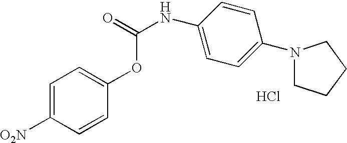

To a stirred solution of 4.9 g (30.4 mmol) of 4-pyrrolidin-1-yl-phenylamine in 70 mL of anhydrous THF at room temperature, was added dropwise a solution of 6.4 g (32 mmol) of 4-nitrophenyl chloroformate in 16 mL of anhydrous THF. After the addition was complete, the mixture was stirred for 1 h and then filtered. The precipitate was washed first with anhydrous THF (2×10 mL) and then with anhydrous DCM (3×10 mL) and dried in vacuo to yield 10 g of an off-white solid. ¹H-NMR (300 MHz, CD₃OD): 10.39 (s, 1H), 8.32 (d, 2H), 7.73 (d, 2H), 7.60 (d, 2H), 7.48 (d, 2H), 3.86-3.68 (bs, 4H), 2.35-2.24 (bs, 4H). LC/MS (ESI): 328 (MH)⁺.

### b. 1-(4-Pyrrolidin-1-yl-phenyl)-3-(1-thieno[3,2-d]pyrimidin-4-yl-pyrrolidin-3-yl)-urea

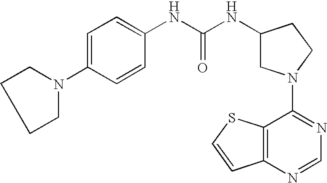

Prepared essentially as described in Example 18 using (4-pyrrolidin-1-yl-phenyl)-carbamic acid 4-nitro-phenyl ester hydrochloride, as described in the previous step, in place of (4-diethylamino-phenyl)-carbamic acid 4-nitro-phenyl ester hydrochloride. Purification was as follows: The combined organic layers were filtered and the filter cake was washed with DCM (1×2 mL) to afford the title compound as a powder (11 mg; 14%). ¹H NMR (400 MHz, CDCl₃) δ 8.40 (s, 1H), 8.20 (d, 1H), 7.90 (br s, 1H), 7.40 (d, 1H), 7.15 (m, 2H), 6.44 (m, 2H), 6.41 (br s, 1H), 4.33 (m, 1H), 4.15-3.80 (br m, 3H), 3.74 (br m, 1H), 3.15 (m, 4H), 2.23 (m, 1H), 1.96 (m, 1H), 1.91 (m, 4H). LC/MS (ESI): calcd mass 408.2, found 409.1 (MH)⁺.

### EXAMPLE 20

### 1-(6-Cyclobutoxy-pyridin-3-yl)-3-(1-thieno[3,2-d]pyrimidin-4-yl-piperidin-4-yl)-urea

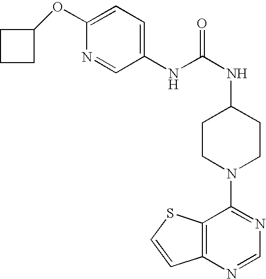

### a. (1-Thieno[3,2-d]pyrimidin-4-yl-piperidin-4-yl)-carbamic acid tert-butyl ester

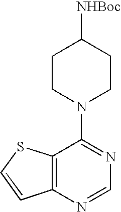

A solution of 4-chlorothieno[3,2-d]pyimidine (400 mg, 2.35 mmol), Piperidin-4-yl-carbamic acid tert-butyl ester (470 mg, 2.35 mmol), diisopropylethylamine (285 mg, 2.82 mmol) in isopropanol (10 mL) was heated to 100° C. for 2 hr. The resulting mixture was cooled to RT, poured into ethyl acetate (50 mL), and washed with water (25 mL). The organic layer was dried over anhydrous sodium sulfate, concentrated, and purified by silica gel chromatography (3% MeOH/EtOAc) to provide the title compound (672 mg, 86% yield). ¹H NMR (400 MHz, CD₃OD) δ 8.34 (s, 1H), 8.02 (d, 1H), 7.36 (d, 1H), 4.76 (m, 2H), 3.72 (m, 1H), 3.38 (m, 2H), 2.02 (m, 2H), 1.58-1.42 (m, 2H), 1.42 (s, 9H). LC/MS (ESI): calcd mass 334.2, found 335.2 (MH)⁺.

### b. 1-Thieno[3,2-d]pyrimidin-4-yl-piperidin-4-ylamine

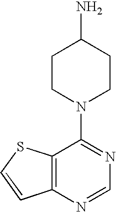

A solution of (1-Thieno[3,2-d]pyrimidin-4-yl-piperidin-4-yl)-carbamic acid tert-butyl ester (672 mg, 2.01 mmol), TFA (5 mL) and CH₂Cl₂ (10 mL) was stirred at RT for 16 h. The reaction mixture was concentrated then diluted with CH₂Cl₂ (100 mL) and sequentially washed with 1N NaOH (50 mL) and brine (50 mL). The organic layer was dried over anhydrous sodium sulfate and concentrated to provide the title compound as an oil (290 mg, 62%). ¹H NMR (400 MHz, CDCl₃) δ 8.58 (s, 1H), 7.72 (d, 1H), 7.42 (d, 1H), 4.74 (m, 2H), 3.23 (m, 2H), 3.02 (m, 1H), 2.02 (m, 2H), 1.42 (m, 4H), 1.42 (s, 9H). LC/MS (ESI): calcd mass 234.1, found 235.1 (MH)⁺.

### c. 1-(6-Cyclobutoxy-pyridin-3-yl)-3-(1-thieno[3,2-d]pyrimidin-4-yl-piperidin-4-yl)-urea

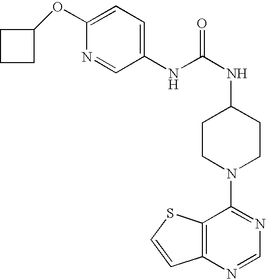

Prepared essentially as described in Example 18b using 1-thieno[3,2-d]pyrimidin-4-yl-piperidin-4-ylamine, prepared as described in the previous step, in place of 1-thieno[3,2-d]pyrimidin-4-yl-pyrrolidin-3-ylamine hydrochloride, and using (6-cyclobutoxy-pyridin-3-yl)-carbamic acid 4-nitro-phenyl ester (Example 15c) in place of (4-diethylamino-phenyl)-carbamic acid 4-nitro-phenyl ester hydrochloride. Also, 600 μL 95:5 CHCl₃/MeOH was used in place of 300 μL CHCl₃ to improve the solubility of the reaction components. Purification was as follows: The crude reaction was diluted with DCM (2 mL) and filtered. The filter cake was washed with DCM (1×2 mL) and dried to afford the title compound as a solid. ¹H NMR (400 MHz, DMSO-d6) δ 8.49 (s, 1H), 8.26 (br s, 1H), 8.21 (d, 1H), 8.07 (d, 1H), 7.74 (dd, 1H), 7.44 (d, 1H), 6.67 (d, 1H), 6.25 (d, 1H), 5.03 (p, 1H), 4.57 (m, 2H), 3.85 (m, 1H), 3.40 (m, 2H), 2.35 (m, 2H), 2.06-1.93 (m, 4H), 1.75 (m, 1H), 1.61 (m, 1H), 1.45 (m, 2H). LC/MS (ESI): calcd mass 424.2, found 425.1 (MH)⁺.

### EXAMPLE 21

### 1-(4-Cyclohexyl-phenyl)-3-(1-thieno[3,2-d]pyrimidin-4-yl-piperidin-4-yl)-urea

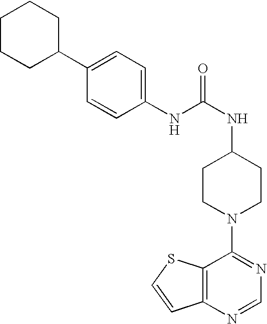

Prepared essentially as described in Example 18 using 1-thieno[3,2-d]pyrimidin-4-yl-piperidin-4-ylamine, prepared as described in Example 20b, in place of 1-thieno[3,2-d]pyrimidin-4-yl-pyrrolidin-3-ylamine hydrochloride, and using using (4-cyclohexyl-phenyl)-carbamic acid 4-nitro-phenyl ester (Example 16a) in place of (4-diethylamino-phenyl)-carbamic acid 4-nitro-phenyl ester hydrochloride. The title compound was purified as described in Example 20c. ¹H NMR (400 MHz, DMSO-d6) δ 8.49 (s, 1H), 8.24 (br s, 1H), 8.21 (d, 1H), 7.45 (d, 1H), 7.27 (m, 2H), 7.06 (m, 2H), 6.16 (d, 1H), 4.56 (m, 2H), 3.85 (m, 1H), 3.41 (m, 2H), 2.39 (m, 1H), 1.98 (m, 2H), 1.80-1.65 (m, 5H), 1.45 (m, 2H), 1.34 (m, 4H), 1.21 (m, 1H). LC/MS (ESI): calcd mass 435.2, found 436.1 (MH)⁺.

### EXAMPLE 22

### 1-(4-Phenoxy-phenyl)-3-(1-thieno[3,2-d]pyrimidin-4-yl-piperidin-4-yl)-urea

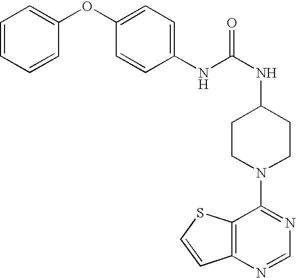

4-Phenoxyphenyl isocyanate (35 mg, 170 μmol) was added to a solution of 1-thieno[3,2-d]pyrimidin-4-yl-piperidin-4-ylamine (35 mg, 150 μmol) (Example 20b) in DCM (300 μL). The solution was stirred at rt overnight, at which point it became a slurry. The reaction was then partitioned with DCM (2 mL) and 2.0M K₂CO₃ (2 mL), the aqueous layer was extracted with 9:1 DCM/MeOH (2×2 mL), and the combined organic layers were filtered. The clear filtrate was dried (Na₂SO₄), concentrated, and purified by C18 HPLC followed by a bicarbonate solid phase extraction cartridge to afford the title compound (46.6 mg, 70%). ¹H NMR (400 MHz, CDCl₃) δ 8.57 (s, 1H), 7.72 (d, 1H), 7.42 (d, 1H), 7.33 (m, 2H), 7.22 (m, 2H), 7.10 (m, 1H), 6.97 (m, 4H), 6.19 (br s, 1H), 4.76 (m, 2H), 4.54 (d, 1H), 4.08 (m, 1H), 3.30 (m, 2H), 2.16 (m, 2H), 1.45 (m, 2H). LC/MS (ESI): calcd mass 445.2, found 446.1 (MH)⁺.

### EXAMPLE 23

### 1-(4-Pyrrolidin-1-yl-phenyl)-3-(1-thieno[3,2-d]pyrimidin-4-yl-piperidin-4-yl)-urea

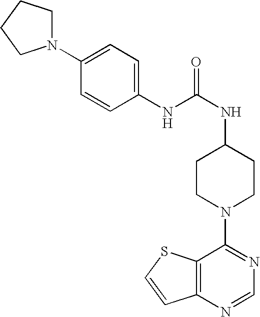

Prepared essentially as described in Example 18 using 1-thieno[3,2-d]pyrimidin-4-yl-piperidin-4-ylamine (Example 20b) instead of 1-thieno[3,2-d]pyrimidin-4-yl-pyrrolidin-3-ylamine hydrochloride, and using (4-pyrrolidin-1-yl-phenyl)-carbamic acid 4-nitro-phenyl ester hydrochloride (Example 19a) instead of (4-diethylamino-phenyl)-carbamic acid 4-nitro-phenyl ester hydrochloride. In addition, the reaction solvent was DMSO-d₆ (300 μL) instead of CHCl₃ (300 μL). ¹H NMR (400 MHz, CDCl₃) δ 8.55 (s, 1H), 7.71 (d, 1H), 7.41 (d, 1H), 7.03 (m, 2H), 6.49 (m, 2H), 5.85 (br s, 1H), 4.70 (m, 2H), 4.40 (d, 1H), 4.05 (m, 1H), 3.32-3.22 (m, 6H), 2.10 (m, 2H), 2.00 (m, 4H), 1.37 (m, 2H). LC/MS (ESI): calcd mass 422.2, found 423.1 (MH)⁺.

### EXAMPLE 24

### 1-(4-Morpholin-4-yl-phenyl)-3-(1-thieno[3,2-d]pyrimidin-4-yl-piperidin-4-yl)-urea

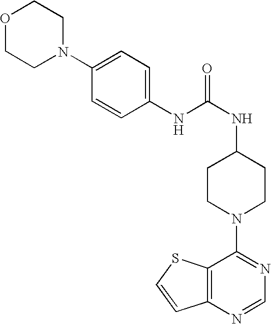

Prepared essentially as described in Example 20c, except (4-morpholin-4-yl-phenyl)-carbamic acid 4-nitro-phenyl ester hydrochloride (Example 14a) used in place of (6-cyclobutoxy-pyridin-3-yl)-carbamic acid 4-nitro-phenyl ester. ¹H NMR (400 MHz, CDCl₃) δ 8.56 (s, 1H), 7.71 (d, 1H), 7.41 (d, 1H), 7.13 (m, 2H), 6.86 (m, 2H), 6.07 (br s, 1H), 4.73 (m, 2H), 4.51 (d, 1H), 4.06 (m, 1H), 3.85 (m, 4H), 3.28 (m, 2H), 3.12 (m, 4H), 2.13 (m, 2H), 1.41 (m, 2H). LC/MS (ESI): calcd mass 438.2, found 439.1 (MH)⁺.

### EXAMPLE 25

### (6-Cyclobutoxy-pyridin-3-yl)-carbamic acid 1-thieno[3,2-d]pyrimidin-4-yl-pyrrolidin-3-yl ester

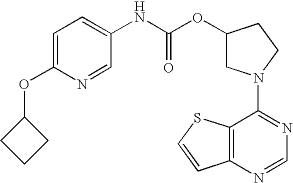

### a. 1-Thieno[3,2-d]pyrimidin-4-yl-pyrrolidin-3-ol

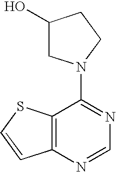

4-Chloro-thieno[3,2-d]pyrimidine (0.985 g, 5.78 mmol) was added to a mixture of racemic 3-pyrrolidinol (0.527 g, 6.06 mmol), DIPEA (1.10 mL, 6.31 mmol), and DMSO (1.5 mL). The mixture was stirred at “rt” for 2 min, during which time it spontaneously warmed and became a nearly homogeneous solution. The reaction was then stirred at 100° C. for 10 min, and the resulting homogeneous dark reddish-brown solution was allowed to cool to rt and then shaken with water (˜17 mL) before extracting with EtOAc (1×20 mL). The organic layer was washed with 4M NaCl (1×20 mL), dried (Na₂SO₄), and concentrated to give 150 mg of title compound. Additional title compound was obtained as follows: The aqueous layers were combined (˜40 mL) and extracted with EtOAc (1×300 mL), and the organic layer was dried (Na₂SO₄) and concentrated to give a powder. The two organic extract-derived powders were combined to give 911 mg of the title compound (71%). ¹H NMR (400 MHz, DMSO-d6) δ 8.38 (s, 1H), 8.18 (d, 1H), 7.39 (d, 1H), 5.12 (br s, 1H), 4.43 (br s, 1H), 4.05-3.68 (br m, 4H), 2.12-1.90 (m, 2H).

### b. (6-Cyclobutoxy-pyridin-3-yl)-carbamic acid 1-thieno[3,2-d]pyrimidin-4-yl-pyrrolidin-3-yl ester

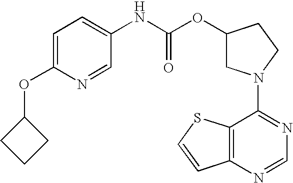

(6-Cyclobutoxy-pyridin-3-yl)-carbamic acid 4-nitro-phenyl ester (79 mg, 240 μmol) (Example 15c) was added to a homogeneous rt solution of 1-thieno[3,2-d]pyrimidin-4-yl-pyrrolidin-3-ol (44 mg, 200 μmol), as prepared in the previous step, DIPEA (108 μL, 620 μmol), and DMSO (200 μL). The resulting mixture was stirred at 100° C. for 20 min to give a homogeneous solution that was allowed to cool to rt. The reaction was then partitioned with 2M K₂CO₃ (2 mL) and DCM (2 mL), the aqueous layer was extracted with DCM (1×2 mL), and the combined organic layers were dried (Na₂SO₄) and concentrated. The residue was purified by C18 HPLC followed by solid phase extraction through a bicarbonate cartridge to afford the title compound (23.0 mg, 28%). ¹H NMR (400 MHz, CDCl₃) δ 8.53 (s, 1H), 8.04 (s, 1H), 7.78 (d, 1H), 7.74 (d, 1H), 7.41 (d, 1H), 6.83 (br s, 1H), 6.68 (d, 1H), 5.52 (m, 1H), 5.10 (p, 1H), 4.16 (m, 2H), 4.11-3.91 (m, 2H), 2.49-2.24 (m, 3H), 2.11 (m, 2H), 1.82 (m, 1H), 1.65 (m, 2H). LC/MS (ESI): calcd mass 411.1, found 412.1 (MH)⁺.

### EXAMPLE 26

### (4-Phenoxy-phenyl)-carbamic acid 1-thieno[3,2-d]pyrimidin-4-yl-pyrrolidin-3-yl ester

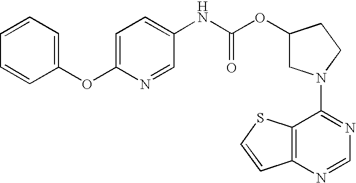

Prepared essentially as described for Example 25, except 4-phenoxyphenyl isocyanate was used in place of (6-cyclobutoxy-pyridin-3-yl)-carbamic acid 4-nitro-phenyl ester, 1.1 eq DIPEA (38 μL) was used instead of 3.1 eq DIPEA, and the reaction was stirred at rt for 1 hr before stirring at 100° C. for 20 min. ¹H NMR (400 MHz, CDCl₃) δ 8.54 (s, 1H), 7.75 (d, 1H), 7.41 (d, 1H), 7.38-7.29 (m, 4H), 7.08 (m, 1H), 6.98 (m, 4H), 6.81 (br s, 1H), 5.54 (m, 1H), 4.17 (m, 2H), 4.04 (m, 2H), 2.35 (m, 2H). LC/MS (ESI): calcd mass 432.1, found 433.1 (MH)⁺.

### EXAMPLE 27

### (4-Pyrrolidin-1-yl-phenyl)-carbamic acid 1-thieno[3,2-d]pyrimidin-4-yl-pyrrolidin-3-yl ester

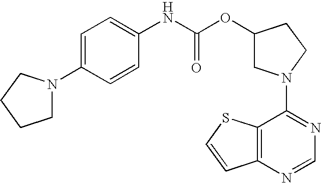

Prepared essentially as described for Example 25, using (4-pyrrolidin-1-yl-phenyl)-carbamic acid 4-nitro-phenyl ester hydrochloride (Example 19a) in place of (6-cyclobutoxy-pyridin-3-yl)-carbamic acid 4-nitro-phenyl ester. ¹H NMR (400 MHz, CDCl₃) δ 8.55 (s, 1H), 7.75 (d, 1H), 7.41 (d, 1H), 7.20 (m, 2H), 6.51 (m, 2H), 6.37 (br s, 1H), 5.52 (m, 1H), 4.21-3.95 (m, 4H), 3.25 (m, 4H), 2.32 (m, 2H), 1.99 (m, 4H). LC/MS (ESI): calcd mass 409.2, found 410.1 (MH)⁺.

### EXAMPLE 28

### (4-Morpholin-4-yl-phenyl)-carbamic acid 1-thieno[3,2-d]pyrimidin-4-yl-pyrrolidin-3-yl ester

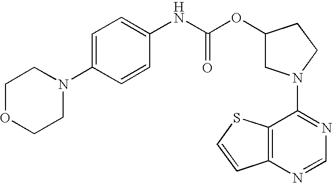

Prepared essentially as described for Example 25, using (4-morpholin-4-yl-phenyl)-carbamic acid 4-nitro-phenyl ester hydrochloride (Example 14a) in place of (6-cyclobutoxy-pyridin-3-yl)-carbamic acid 4-nitro-phenyl ester. ¹H NMR (400 MHz, CDCl₃) δ 8.54 (s, 1H), 7.75 (d, 1H), 7.41 (d, 1H), 7.28 (m, 2H), 6.87 (m, 2H), 6.63 (br s, 1H), 5.52 (m, 1H), 4.21-3.93 (m, 4H), 3.85 (m, 4H), 3.10 (m, 4H), 2.41-2.24 (m, 2H). LC/MS (ESI): calcd mass 425.1, found 426.1 (MH)⁺.

### EXAMPLE 29

### (4-Diethylamino-phenyl)-carbamic acid 1-thieno[3,2-d]pyrimidin-4-yl-pyrrolidin-3-yl ester

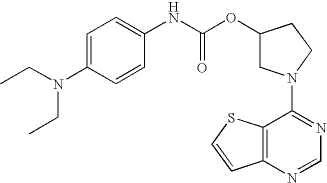

Prepared essentially as described for Example 25, using (4-diethylamino-phenyl)-carbamic acid 4-nitro-phenyl ester hydrochloride (Example 18a) in place of (6-cyclobutoxy-pyridin-3-yl)-carbamic acid 4-nitro-phenyl ester. ¹H NMR (400 MHz, CDCl₃) δ 8.53 (s, 1H), 7.73 (d, 1H), 7.40 (d, 1H), 7.19 (m, 2H), 6.63 (m, 3H), 5.51 (m, 1H), 4.19-3.90 (m, 4H), 3.31 (q, 4H), 2.40-2.21 (m, 2H), 1.12 (t, 6H). LC/MS (ESI): calcd mass 411.2, found 412.2 (MH)⁺.

Biological Activity of FLT3 Inhibitors of Formula I′ and Formula II′

The following representative assays were performed in determining the biological activities of the FLT3 inhibitors of Formula I′ and Formula II′. They are given to illustrate the invention in a non-limiting fashion.

In Vitro Assays

The following representative in vitro assays were performed in determining the biological activities of the FLT3 inhibitors of Formula I′ and Formula II′ within the scope of the invention. They are given to illustrate the invention in a non-limiting fashion.

Inhibition of FLT3 enzyme activity, MV4-11 proliferation and Baf3-FLT3 phosphorylation exemplify the specific inhibition of the FLT3 enzyme and cellular processes that are dependent on FLT3 activity. Inhibition of Baf3 cell proliferation is used as a test of FLT3, c-Kit and TrkB independent cytotoxicity of compounds within the scope of the invention. All of the examples herein show significant and specific inhibition of the FLT3 kinase and FLT3-dependent cellular responses. Examples herein also show specific inhibition of the TrkB and c-kit kinase in an enzyme activity assay. The FLT3 inhibitor compounds are also cell permeable.

FLT3 Fluorescence Polarization Kinase Assay

To determine the activity of the FLT3 inhibitors of Formula I′ and Formula II′ in an in vitro kinase assay, inhibition of the isolated kinase domain of the human FLT3 receptor (a.a. 571-993) was performed using the following fluorescence polarization (FP) protocol. The FLT3 FP assay utilizes the fluorescein-labeled phosphopeptide and the anti-phosphotyrosine antibody included in the Panvera Phospho-Tyrosine Kinase Kit (Green) supplied by Invitrogen. When FLT3 phosphorylates polyGlu₄Tyr, the fluorescein-labeled phosphopeptide is displaced from the anti-phosphotyrosine antibody by the phosphorylated poly Glu₄Tyr, thus decreasing the FP value. The FLT3 kinase reaction is incubated at room temperature for 30 minutes under the following conditions: 10 nM FLT3571-993, 20 ug/mL poly Glu₄Tyr, 150 uM ATP, 5 mM MgCl₂, 1% compound in DMSO. The kinase reaction is stopped with the addition of EDTA. The fluorescein-labeled phosphopeptide and the anti-phosphotyrosine antibody are added and incubated for 30 minutes at room temperature.

All data points are an average of triplicate samples. Inhibition and IC₅₀ data analysis was done with GraphPad Prism using a non-linear regression fit with a multiparamater, sigmoidal dose-response (variable slope) equation. The IC₅₀ for kinase inhibition represents the dose of a compound that results in a 50% inhibition of kinase activity compared to DMSO vehicle control.

Inhibition of MV4-11 and Baf3 Cell Proliferation

To assess the cellular potency of the FLT3 inhibitors of Formula I′ and Formula II′, FLT3 specific growth inhibition was measured in the leukemic cell line MV4-11 (ATCC Number: CRL-9591). MV4-11 cells are derived from a patient with childhood acute myelomonocytic leukemia with an 11q23 translocation resulting in a MLL gene rearrangement and containing an FLT3-ITD mutation (AML subtype M4)(see Drexler H G. The Leukemia-Lymphoma Cell Line Factsbook. Academic Pres: San Diego, Calif., 2000 and Quentmeier H, Reinhardt J, Zaborski M, Drexler H G. FLT3 mutations in acute myeloid leukemia cell lines. Leukemia. 2003 January; 17:120-124.). MV4-11 cells cannot grow and survive without active FLT31TD.

The IL-3 dependent, murine b-cell lymphoma cell line, BaB3, were used as a control to confirm the selectivity of the FLT3 inhibitor compounds by measuring non-specific growth inhibition by the FLT3 inhibitor compounds.

To measure proliferation inhibition by test compounds, the luciferase based CellTiterGlo reagent (Promega), which quantifies total cell number based on total cellular ATP concentration, was used. Cells are plated at 10,000 cells per well in 100 ul of in RPMI media containing penn/strep, 10% FBS and 1 ng/ml GM-CSF or 1 ng/ml IL-3 for MV4-11 and Baf3 cells respectively.

Compound dilutions or 0.1% DMSO (vehicle control) are added to cells and the cells are allowed to grow for 72 hours at standard cell growth conditions (37° C., 5% CO₂). For activity measurements in MV4-11 cells grown in 50% plasma, cells were plated at 10,000 cells per well in a 1:1 mixture of growth media and human plasma (final volume of 100 μL). To measure total cell growth an equal volume of CellTiterGlo reagent was added to each well, according to the manufacturer's instructions, and luminescence was quantified. Total cell growth was quantified as the difference in luminescent counts (relative light units, RLU) of cell number at Day 0 compared to total cell number at Day 3 (72 hours of growth and/or compound treatment). One hundred percent inhibition of growth is defined as an RLU equivalent to the Day 0 reading. Zero percent inhibition was defined as the RLU signal for the DMSO vehicle control at Day 3 of growth. All data points are an average of triplicate samples. The IC₅₀ for growth inhibition represents the dose of a compound that results in a 50% inhibition of total cell growth at day 3 of the DMSO vehicle control. Inhibition and IC₅₀ data analysis was done with GraphPad Prism using a non-linear regression fit with a multiparamater, sigmoidal dose-response (variable slope) equation.

MV4-11 cells express the FLT3 internal tandem duplication mutation, and thus are entirely dependent upon FLT3 activity for growth. Strong activity against the MV4-11 cells is anticipated to be a desirable quality of the invention. In contrast, the Baf3 cell proliferation is driven by the cytokine IL-3 and thus are used as a non-specific toxicity control for test compounds. All compound examples in the present invention showed <50% inhibition at a 3 uM dose (data is not included), suggesting that the compounds are not cytotoxic and have good selectivity for FLT3.

Cell-Based FLT3 Receptor Elisa

Specific cellular inhibition of FLT ligand-induced wild-type FLT3 phosphorylation was measured in the following manner: Baf3 FLT3 cells overexpressing the FLT3 receptor were obtained from Dr. Michael Heinrich (Oregon Health and Sciences University). The Baf3 FLT3 cell lines were created by stable transfection of parental Baf3 cells (a murine B cell lymphoma line dependent on the cytokine IL-3 for growth) with wild-type FLT3. Cells were selected for their ability to grow in the absence of IL-3 and in the presence of FLT3 ligand.

Baf3 cells were maintained in RPMI 1640 with 10% FBS, penn/strep and 10 ng/ml FLT ligand at 37° C., 5% CO₂. To measure direct inhibition of the wild-type FLT3 receptor activity and phosphorylation a sandwich ELISA method was developed similar to those developed for other RTKs (see Sadick, M D, Sliwkowski, M X, Nuijens, A, Bald, L, Chiang, N, Lofgren, J A, Wong W L T. Analysis of Heregulin-Induced ErbB2 Phosphorylation with a High-Throughput Kinase Receptor Activation Enzyme-Linked Immunsorbent Assay, Analytical Biochemistry. 1996; 235:207-214 and Baumann C A, Zeng L, Donatelli R R, Maroney A C. Development of a quantitative, high-throughput cell-based enzyme-linked immunosorbent assay for detection of colony-stimulating factor-1 receptor tyrosine kinase inhibitors. J Biochem Biophys Methods. 2004; 60:69-79.). 200 μL of Baf3FLT3 cells (1×10⁶/mL) were plated in 96 well dishes in RPMI 1640 with 0.5% serum and 0.01 ng/mL IL-3 for 16 hours prior to 1 hour compound or DMSO vehicle incubation. Cells were treated with 100 ng/mL Flt ligand (R&D Systems Cat# 308-FK) for 10 min. at 37° C. Cells were pelleted, washed and lysed in 100 ul lysis buffer (50 mM Hepes, 150 mM NaCl, 10% Glycerol, 1% Triton-X-100, 10 mM NaF, 1 mM EDTA, 1.5 mM MgCl₂, 10 mM NaPyrophosphate) supplemented with phosphatase (Sigma Cat# P2850) and protease inhibitors (Sigma Cat #P8340). Lysates were cleared by centrifugation at 1000×g for 5 minutes at 4° C. Cell lysates were transferred to white wall 96 well microtiter (Costar #9018) plates coated with 50 ng/well anti-FLT3 antibody (Santa Cruz Cat# sc-480) and blocked with SeaBlock reagent (Pierce Cat#37527). Lysates were incubated at 4° C. for 2 hours. Plates were washed 3× with 200 ul/well PBS/0.1% Triton-X-100. Plates were then incubated with 1:8000 dilution of HRP-conjugated anti-phosphotyrosine antibody (Clone 4G10, Upstate Biotechnology Cat#16-105) for 1 hour at room temperature. Plates were washed 3× with 200 ul/well PBS/0.1% Triton-X-100. Signal detection with Super Signal Pico reagent (Pierce Cat#37070) was done according to manufacturer's instruction with a Berthold microplate luminometer. All data points are an average of triplicate samples. The total relative light units (RLU) of Flt ligand stimulated FLT3 phosphorylation in the presence of 0.1% DMSO control was defined as 0% inhibition and 100% inhibition was the total RLU of lysate in the basal state. Inhibition and IC₅₀ data analysis was done with GraphPad Prism using a non-linear regression fit with a multiparamater, sigmoidal dose-response (variable slope equation. Biological Data

Biological Data for FLT3

The activity of representative FLT3 inhibitor compounds is presented in the charts hereafter. All activities are in μM and have the following uncertainties: FLT3 kinase: ±10%; MV4-11 and Baf3-FLT3: ±20%.

| | | FLT3 | | BaF3 |
|---|---|---|---|---|
| | | Kinase | MV4- | ELISA |
| No. | Compound Name | (μM) | 11(μM) | (μM) |
| 1 | (4-Isopropyl-phenyl)-carbamic acid 1-thieno[2,3- | 0.102 | 7.200 | nd |
| | d]pyrimidin-4-yl-piperidin-4-yl ester | | | |
| 2 | (4-Isopropoxy-phenyl)-carbamic acid 1-thieno[2,3- | 2.09 | >10 | nd |
| | d]pyrimidin-4-yl-piperidin-4-yl ester | | | |
| 3 | (4-Isopropyl-phenyl)-carbamic acid 1-thieno[2,3- | 0.227 | 2.4 | nd |
| | d]pyrimidin-4-yl-pyrrolidin-3-yl ester | | | |
| 4 | (4-Isopropoxy-phenyl)-carbamic acid 1-thieno[2,3- | 0.761 | >10 | nd |
| | d]pyrimidin-4-yl-pyrrolidin-3-yl ester | | | |
| 5 | (4-Isopropyl-phenyl)-carbamic acid 1-thieno[3,2- | 0.064 | 1.3 | 0.022 |
| | d]pyrimidin-4-yl-pyrrolidin-3-yl ester | | | |
| 6 | (4-Isopropoxy-phenyl)-carbamic acid 1-thieno[3,2- | 0.208 | 6.5 | nd |
| | d]pyrimidin-4-yl-pyrrolidin-3-yl ester | | | |
| 7 | (4-Isopropyl-phenyl)-carbamic acid 1-thieno[3,2- | 0.14 | 4.3 | nd |
| | d]pyrimidin-4-yl-piperidin-4-yl ester | | | |
| 8 | (4-Isopropoxy-phenyl)-carbamic acid 1-thieno[3,2- | 3.03 | 3.6 | nd |
| | d]pyrimidin-4-yl-piperidin-4-yl ester | | | |
| 9 | 1-(4-Isopropyl-phenyl)-3-(1-thieno[2,3- | 0.041 | 1.3 | 0.055 |
| | d]pyrimidin-4-yl-pyrrolidin-3-yl)-urea | | | |
| 10 | 1-(4-Isopropoxy-phenyl)-3-(1-thieno[2,3- | 0.365 | 3.6 | nd |
| | d]pyrimidin-4-yl-pyrrolidin-3-yl)-urea | | | |
| 11 | 1-(4-Isopropyl-phenyl)-3-(1-thieno[3,2- | 0.077 | 0.671 | 0.108 |
| | d]pyrimidin-4-yl-pyrrolidin-3-yl)-urea | | | |
| 12 | 1-(4-Isopropoxy-phenyl)-3-(1-thieno[3,2- | 0.296 | 2.5 | nd |
| | d]pyrimidin-4-yl-pyrrolidin-3-yl)-urea | | | |
| 13 | 1-(4-Phenoxy-phenyl)-3-(1-thieno[3,2-d]pyrimidin- | nd | 0.881 | >5 |
| | 4-yl-pyrrolidin-3-yl)-urea | | | |
| 14 | 1-(4-Morpholin-4-yl-phenyl)-3-(1-thieno[3,2- | nd | >5 | nd |
| | d]pyrimidin-4-yl-pyrrolidin-3-yl)-urea | | | |
| 15 | 1-(6-Cyclobutoxy-pyridin-3-yl)-3-(1-thieno[3,2- | nd | 0.983 | 1.9 |
| | d]pyrimidin-4-yl-pyrrolidin-3-yl)-urea | | | |
| 16 | 1-(4-Cyclohexyl-phenyl)-3-(1-thieno[3,2- | nd | 5.2 | nd |
| | d]pyrimidin-4-yl-pyrrolidin-3-yl)-urea | | | |
| 17 | 1-(4-Bromo-phenyl)-3-(1-thieno[3,2-d]pyrimidin- | nd | 1.9 | nd |
| | 4-yl-pyrrolidin-3-yl)-urea | | | |
| 18 | 1-(4-Diethylamino-phenyl)-3-(1-thieno[3,2- | nd | 0.757 | 0.895 |
| | d]pyrimidin-4-yl-pyrrolidin-3-yl)-urea | | | |
| 19 | 1-(4-Pyrrolidin-1-yl-phenyl)-3-(1-thieno[3,2- | nd | 0.85 | 2.9 |
| | d]pyrimidin-4-yl-pyrrolidin-3-yl)-urea | | | |
| 20 | 1-(6-Cyclobutoxy-pyridin-3-yl)-3-(1-thieno[3,2- | nd | 1.5 | nd |
| | d]pyrimidin-4-yl-piperidin-4-yl)-urea | | | |
| 21 | 1-(4-Cyclohexyl-phenyl)-3-(1-thieno[3,2- | nd | 1.4 | nd |
| | d]pyrimidin-4-yl-piperidin-4-yl)-urea | | | |
| 22 | 1-(4-Phenoxy-phenyl)-3-(1-thieno[3,2-d]pyrimidin- | nd | 3.8 | nd |
| | 4-yl-piperidin-4-yl)-urea | | | |
| 23 | 1-(4-Pyrrolidin-1-yl-phenyl)-3-(1-thieno[3,2- | nd | 3.9 | nd |
| | d]pyrimidin-4-yl-piperidin-4-yl)-urea | | | |
| 24 | 1-(4-Morpholin-4-yl-phenyl)-3-(1-thieno[3,2- | nd | >5 | nd |
| | d]pyrimidin-4-yl-piperidin-4-yl)-urea | | | |
| 25 | (6-Cyclobutoxy-pyridin-3-yl)-carbamic acid 1- | nd | 0.345 | 1.6 |
| | thieno[3,2-d]pyrimidin-4-yl-pyrrolidin-3-yl ester | | | |
| 26 | (4-Phenoxy-phenyl)-carbamic acid 1-thieno[3,2- | nd | 0.953 | 0.39 |
| | d]pyrimidin-4-yl-pyrrolidin-3-yl ester | | | |
| 27 | (4-Pyrrolidin-1-yl-phenyl)-carbamic acid 1- | nd | nd | nd |
| | thieno[3,2-d]pyrimidin-4-yl-pyrrolidin-3-yl ester | | | |
| 28 | (4-Morpholin-4-yl-phenyl)-carbamic acid 1- | nd | 2.1 | nd |
| | thieno[3,2-d]pyrimidin-4-yl-pyrrolidin-3-yl ester | | | |
| 29 | (4-Diethylamino-phenyl)-carbamic acid 1- | nd | nd | nd |
| | thieno[3,2-d]pyrimidin-4-yl-pyrrolidin-3-yl ester | | | |
| * Except where indicated, compound names were derived using nomenclature rules well known to those skilled in the art, by either standard IUPAC nomenclature references, such as Nomenclature of Organic Chemistry, Sections A, B, C, D, F, F and H, (Pergamon Press, Oxford, 1979, Copyright 1979 IUPAC) and A Guide to IUPAC Nomenclature of Organic Compounds (Recommendations 1993), (Blackwell Scientific Publications, 1993, Copyright 1993 IUPAC); or commercially available | | | | |
| # software packages such as Autonom (brand of nomenclature software provided in the ChemDraw Ultra ® office suite marketed by CambridgeSoft.com); and ACD/Index Name ™ (brand of commercial nomenclature software marketed by Advanced Chemistry Development, Inc., Toronto, Ontario). | | | | |

Other FLT3 Inhibitors

Other FLT3 kinase inhibitors which can be employed in accordance with the present include: AG1295 and AG1296; Lestaurtinib (also known as CEP 701, formerly KT-5555, Kyowa Hakko, licensed to Cephalon); CEP-5214 and CEP-7055 (Cephalon); CHIR-258 (Chiron Corp.); EB-10 and IMC-EB10 (ImClone Systems Inc.); GTP 14564 (Merk Biosciences UK). Midostaurin (also known as PKC 412 Novartis AG); MLN 608 (Millennium USA); MLN-518 (formerly CT53518, COR Therapeutics Inc., licensed to Millennium Pharmaceuticals Inc.); MLN-608 (Millennium Pharmaceuticals Inc.); SU-11248 (Pfizer USA); SU-11657 (Pfizer USA); SU-5416 and SU 5614; THRX-165724 (Theravance Inc.); AMI-10706 (Theravance Inc.); VX-528 and VX-680 (Vertex Pharmaceuticals USA, licensed to Novartis (Switzerland), Merck & Co USA); and XL 999 (Exelixis USA).

Formulation

The FLT3 kinase inhibitors and the farnesyl transferase inhibitors of the present invention can be prepared and formulated by methods known in the art, and as described herein. In addition to the preparation and formulations described herein, the farnesyltransferase inhibitors of the present invention can be prepared and formulated into pharmaceutical compositions by methods described in the art, such as the publications cited herein. For example, for the farnesyltransferase inhibitors of formulae (I), (II) and (III) suitable examples can be found in WO-97/21701. The farnesyltransferase inhibitors of formulae (IV), (V), and (VI) can be prepared and formulated using methods described in WO 97/16443, farnesyltransferase inhibitors of formulae (VII) and (VIII) according to methods described in WO 98/40383 and WO 98/49157 and farnesyltransferase inhibitors of formula (IX) according to methods described in WO 00/39082 respectively. Tipifarnib (Zarnestra™, also known as R115777) and its less active enantiomer can be synthesized by methods described in WO 97/21701. Tipifarnib is expected to be available commercially as ZARNESTRA™ in the near future, and is currently available upon request (by contract) from Johnson & Johnson Pharmaceutical Research & Development, L.L.C. (Titusville, N.J.).

Where separate pharmaceutical compositions are utilized, the FLT3 kinase inhibitor or farnesyl transferase inhibitor, as the active ingredient, is intimately admixed with a pharmaceutical carrier according to conventional pharmaceutical compounding techniques, which carrier may take a wide variety of forms depending on the form of preparation desired for administration, e.g., oral or parenteral such as intramuscular. A unitary pharmaceutical composition having both the FLT3 kinase inhibitor and farnesyl transferase inhibitor as active ingredients can be similarly prepared.

In preparing either of the individual compositions, or the unitary composition, in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. In preparation for slow release, a slow release carrier, typically a polymeric carrier, and a compound of the present invention are first dissolved or dispersed in an organic solvent. The obtained organic solution is then added into an aqueous solution to obtain an oil-in-water-type emulsion. Preferably, the aqueous solution includes surface-active agent(s). Subsequently, the organic solvent is evaporated from the oil-in-water-type emulsion to obtain a colloidal suspension of particles containing the slow release carrier and the compound of the present invention.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 0.01 mg to 200 mg/kg of body weight per day. Preferably, the range is from about 0.03 to about 100 mg/kg of body weight per day, most preferably, from about 0.05 to about 10 mg/kg of body weight per day. The compounds may be administered on a regimen of 1 to 5 times per day. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, acetyl alcohol and cellulose acetate.

The liquid forms in which the FLT3 kinase inhibitor and the farnesyl transferase inhibitor individually (or both in the case of a unitary composition) may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. The liquid forms in suitably flavored suspending or dispersing agents may also include the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Advantageously, the FLT3 kinase inhibitor and the farnesyl transferase inhibitor may be administered in a single daily dose (individually or in a unitary composition), or the total daily dosage may be administered in divided doses of two, three or four times daily. Furthermore, compounds for the present invention (individually or in a unitary composition) can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component (the FLT3 kinase inhibitor and the farnesyl transferase inhibitor individually, or together in the case of a unitary composition) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. The daily dosage of the products of the present invention may be varied over a wide range from 1 to 5000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Particularly, the range is from about 0.03 to about 15 mg/kg of body weight per day, and more particularly, from about 0.05 to about 10 mg/kg of body weight per day. The FLT3 kinase inhibitor and the farnesyl transferase inhibitor individually, or together in the case of a unitary composition, may be administered on a regimen up to four or more times per day, preferably of 1 to 2 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The FLT3 kinase inhibitor and the farnesyl transferase inhibitor of the present invention can also be administered (individually or in a unitary composition) in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phosphatidylethanolamines, phophatidylcholines, cardiolipins, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. They may either contain cholesterol or may be cholesterol-free.

The FLT3 kinase inhibitor and the farnesyl transferase inhibitor of the present invention can also be administered (individually or in a unitary composition) locally. Any delivery device, such as intravascular drug delivery catheters, wires, pharmacological stents and endoluminal paving, may be utilized. The delivery system for such a device may comprise a local infusion catheter that delivers the compound at a rate controlled by the administor.

The present invention provides a drug delivery device comprising an intraluminal medical device, preferably a stent, and a therapeutic dosage of the FLT3 kinase inhibitor and the farnesyl transferase inhibitor of the invention. Alternatively, the present invention provides for individual administration of a therapeutic dosage of one or both of the FLT3 kinase inhibitor and the farnesyl transferase inhibitor of the invention by means of a drug delivery device comprising an intraluminal medical device, preferably a stent

The term “stent” refers to any device capable of being delivered by a catheter. A stent is routinely used to prevent vascular closure due to physical anomalies such as unwanted inward growth of vascular tissue due to surgical trauma. It often has a tubular, expanding lattice-type structure appropriate to be left inside the lumen of a duct to relieve an obstruction. The stent has a lumen wall-contacting surface and a lumen-exposed surface. The lumen-wall contacting surface is the outside surface of the tube and the lumen-exposed surface is the inner surface of the tube. The stent can be polymeric, metallic or polymeric and metallic, and it can optionally be biodegradable.

The FLT3 kinase inhibitor and farnesyl transferase inhibitor of the present invention (individually or in a unitary composition) can be incorporated into or affixed to the stent in a number of ways and in utilizing any number of biocompatible materials. In one exemplary embodiment, the compound is directly incorporated into a polymeric matrix, such as the polymer polypyrrole, and subsequently coated onto the outer surface of the stent. The compound elutes from the matrix by diffusion through the polymer. Stents and methods for coating drugs on stents are discussed in detail in the art. In another exemplary embodiment, the stent is first coated with as a base layer comprising a solution of the compound, ethylene-co-vinylacetate, and polybutylmethacrylate. Then, the stent is further coated with an outer layer comprising only polybutylmethacrylate. The outlayer acts as a diffusion barrier to prevent the compound from eluting too quickly and entering the surrounding tissues. The thickness of the outer layer or topcoat determines the rate at which the compound elutes from the matrix. Stents and methods for coating are discussed in detail in WIPO publication WO9632907, U.S. Publication No. 2002/0016625 and references disclosed therein.

To better understand and illustrate the invention and its exemplary embodiments and advantages, reference is made to the following experimental section.

Experimentals

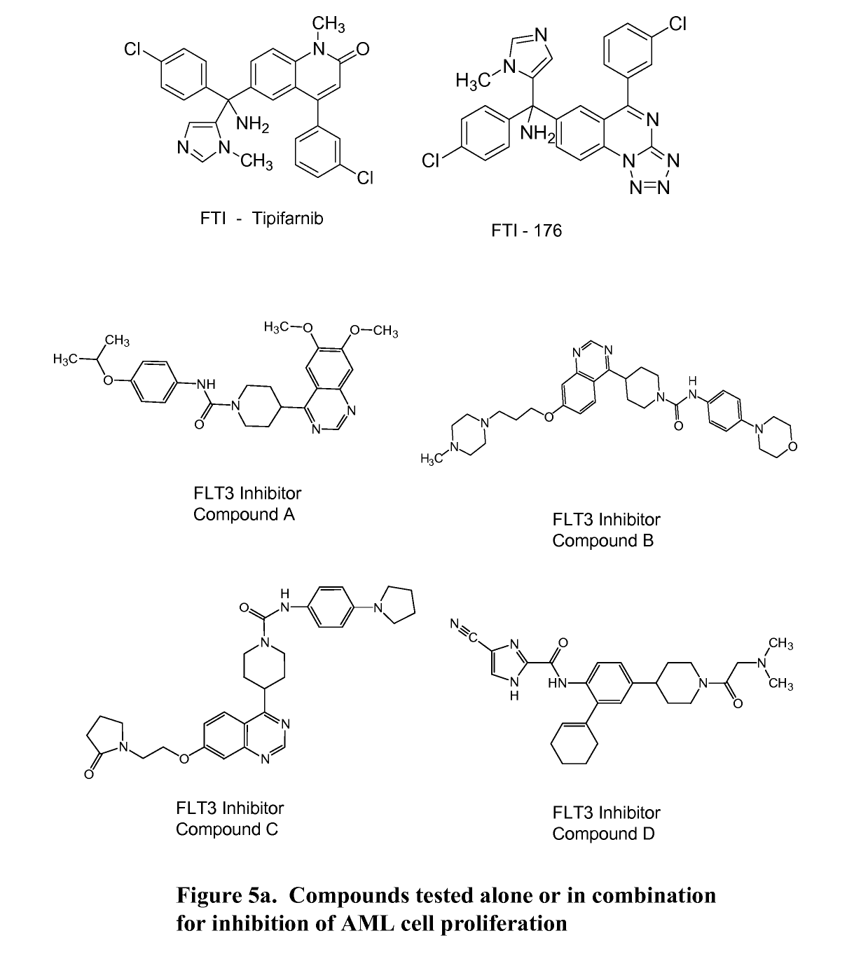

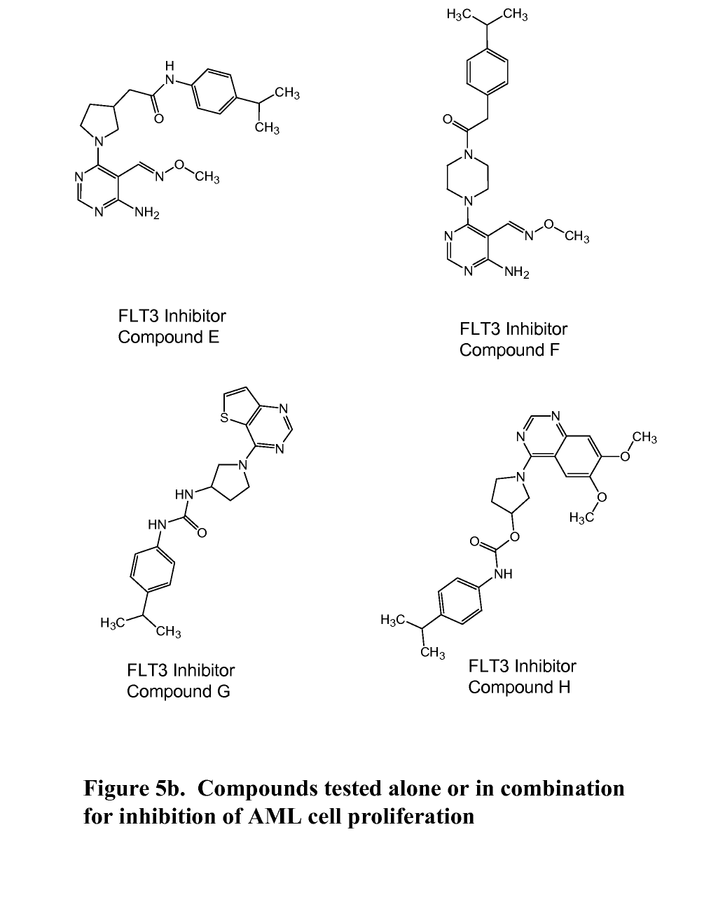

Inhibition of AML cell growth with the combination of an FTI and a FLT3 inhibitor was tested. Two FTIs, Tipifarnib and FTI Compound 176 (“FTI-176), and eight novel FLT3 inhibitors: Compounds A, B, C, D, E, F G and H were used to inhibit the growth of FLT3-dependent cell types in vitro (see FIG. 5 depicting the test compounds).

The cell lines that were tested included those that are dependent on FLT31TD mutant activity for growth (MV4-11 and Baf3-FLT31TD), FLT3 wt activity for growth (Baf3FLT3) and those that grow independent of FLT3 activity (THP-1). MV4-11 (ATCC Number: CRL-9591) cells are derived from a patient with childhood acute myelomonocytic leukemia with an 11q23 translocation resulting in a MLL gene rearrangement and containing an FLT3-ITD mutation (AML subtype M4) (see Drexler H G. The Leukemia-Lymphoma Cell Line Factsbook. Academic Pres: San Diego, Calif., 2000 and Quentmeier H, Reinhardt J, Zaborski M, Drexler H G. FLT3 mutations in acute myeloid leukemia cell lines. Leukemia. 2003 January; 17:120-124.). Baf3-FLT3 and Baf3-FLT31TD cell lines were obtained from Dr. Michael Henrich and the Oregon Health Sciences University. The Baf3 FLT3 cell lines were created by stable transfection of parental Baf3 cells (a murine B cell lymphoma line dependent on the cytokine IL-3 for growth) with either wild-type FLT3 or FLT3 containing the ITD insertion in the juxatamembrane domain of the receptor resulting in its constitutive activation. Cells were selected for their ability to grow in the absence of IL-3 and in either the presence of FLT3 ligand (Baf3-FLT3) or independent of any growth factor (Baf3-ITD). THP-1 (ATCC Number: TIB-202) cells were isolated from a childhood AML patient with an N-Ras mutation and no FLT3 abnormality. Although the cells express a functional FLT3 receptor, THP-1 cells are not dependent on FLT3 activity for viability and growth (data not shown).

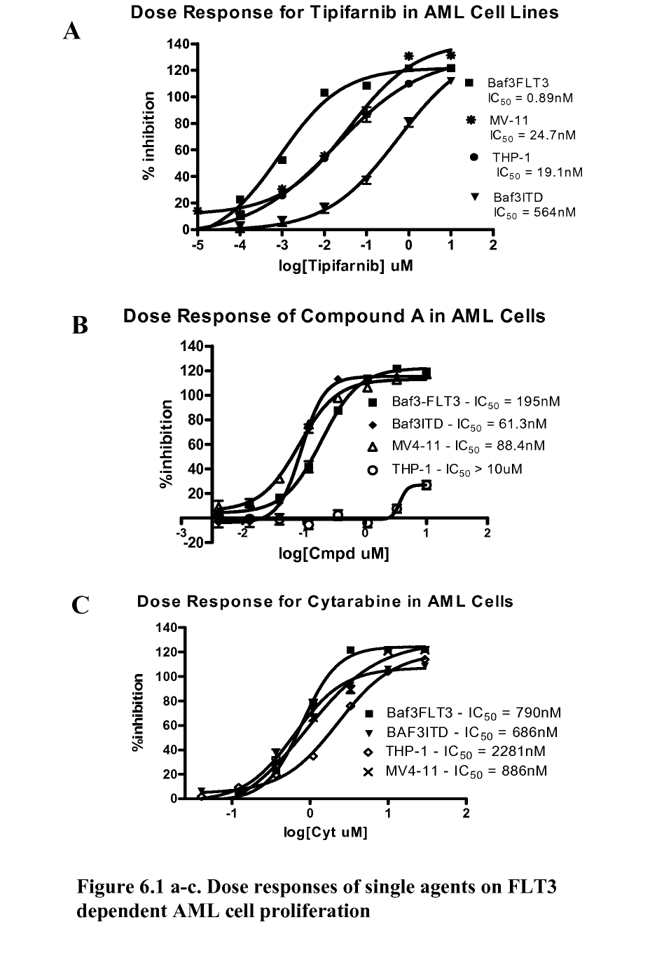

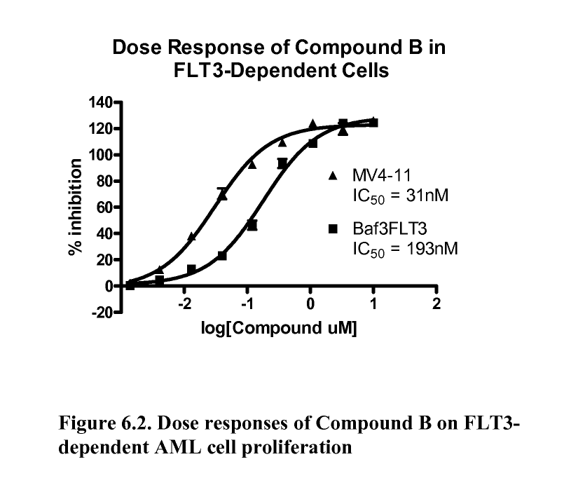

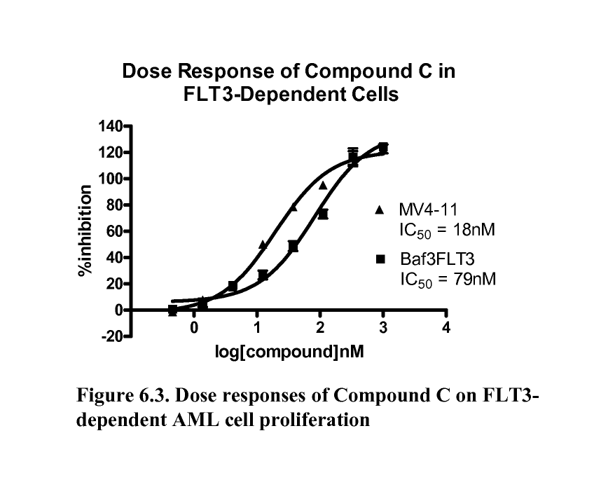

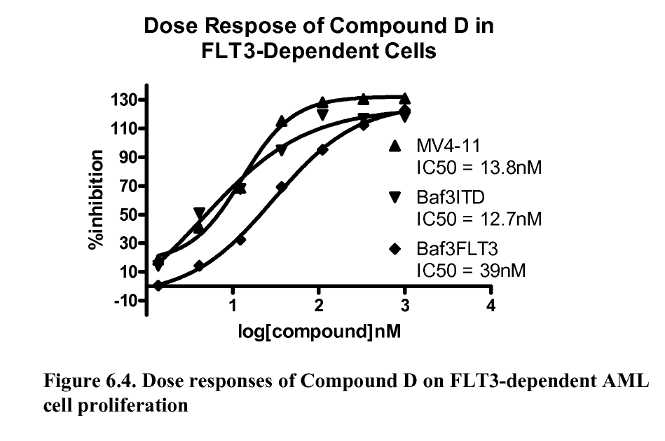

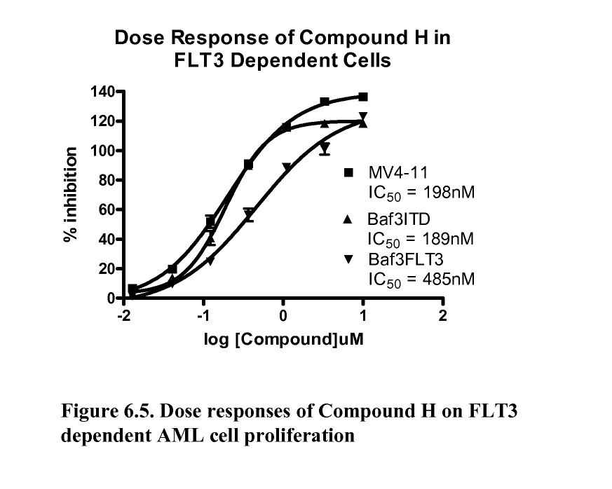

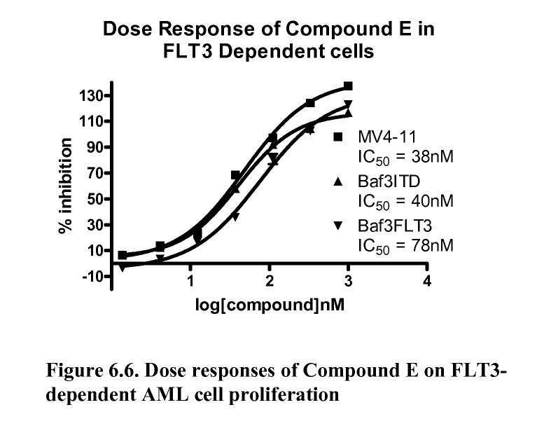

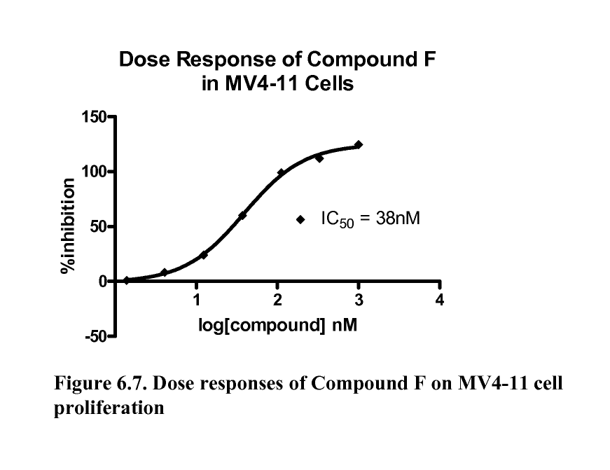

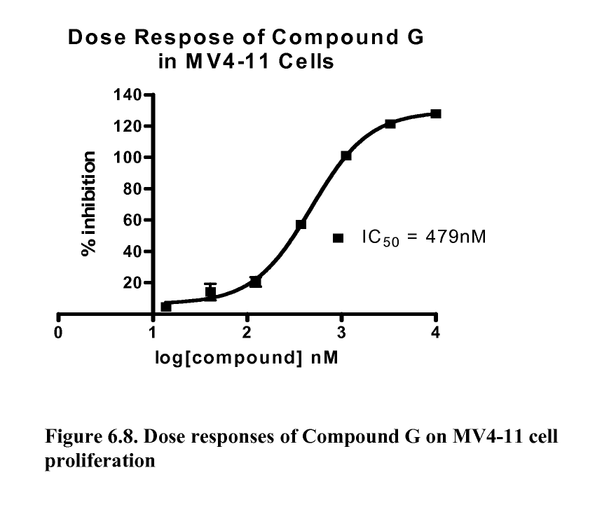

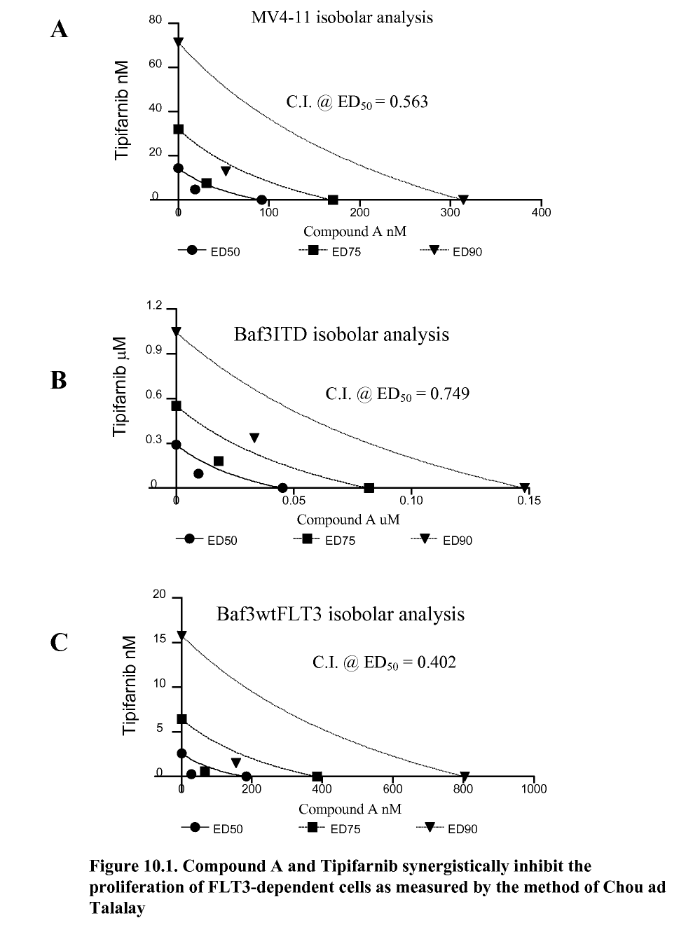

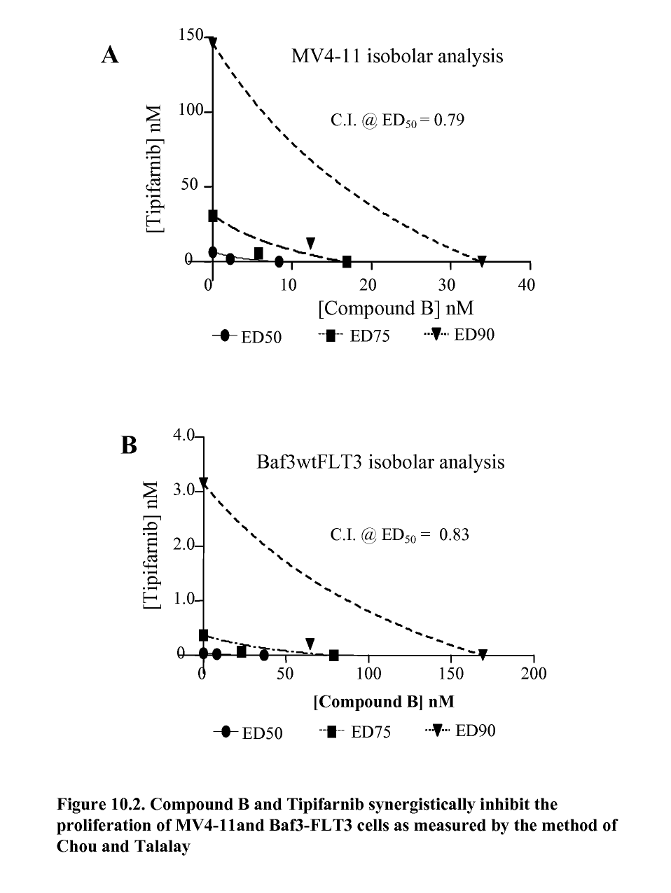

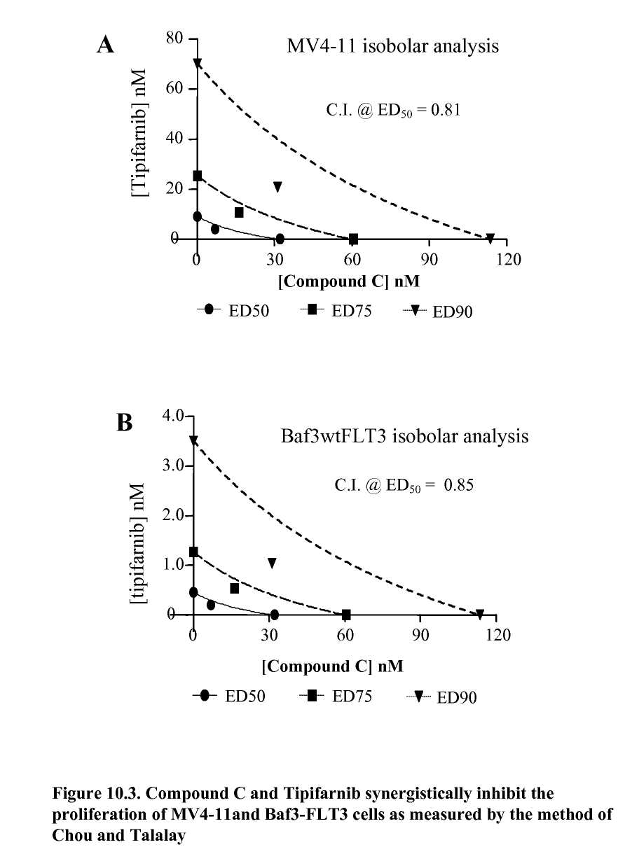

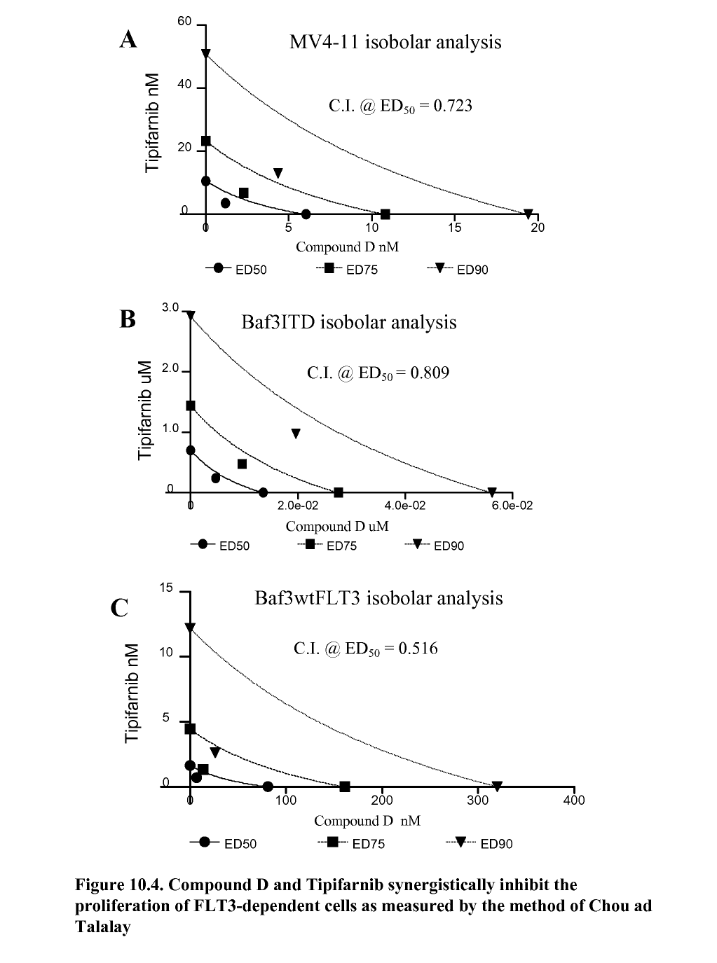

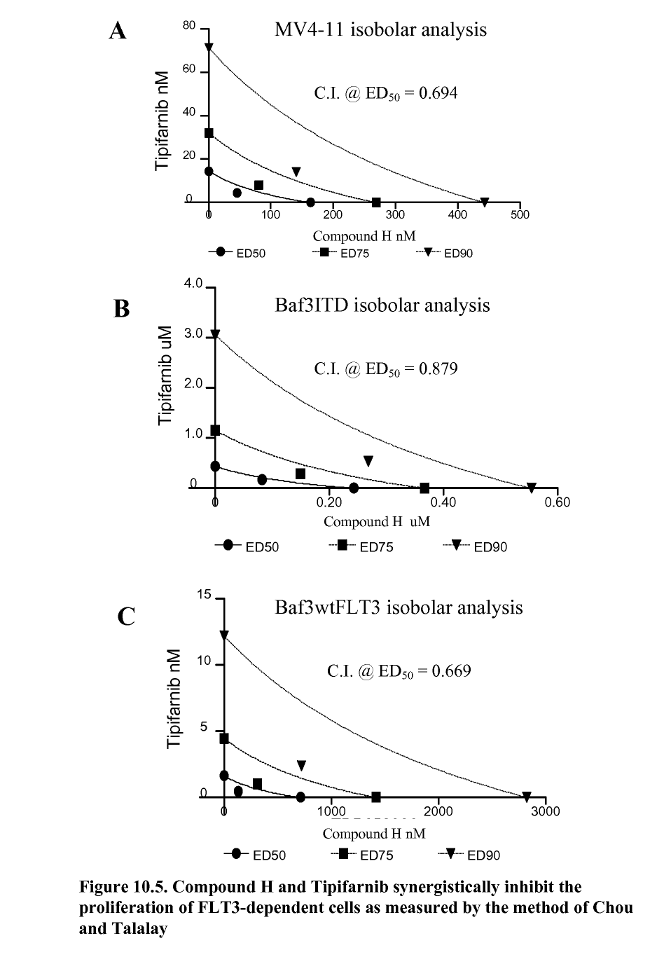

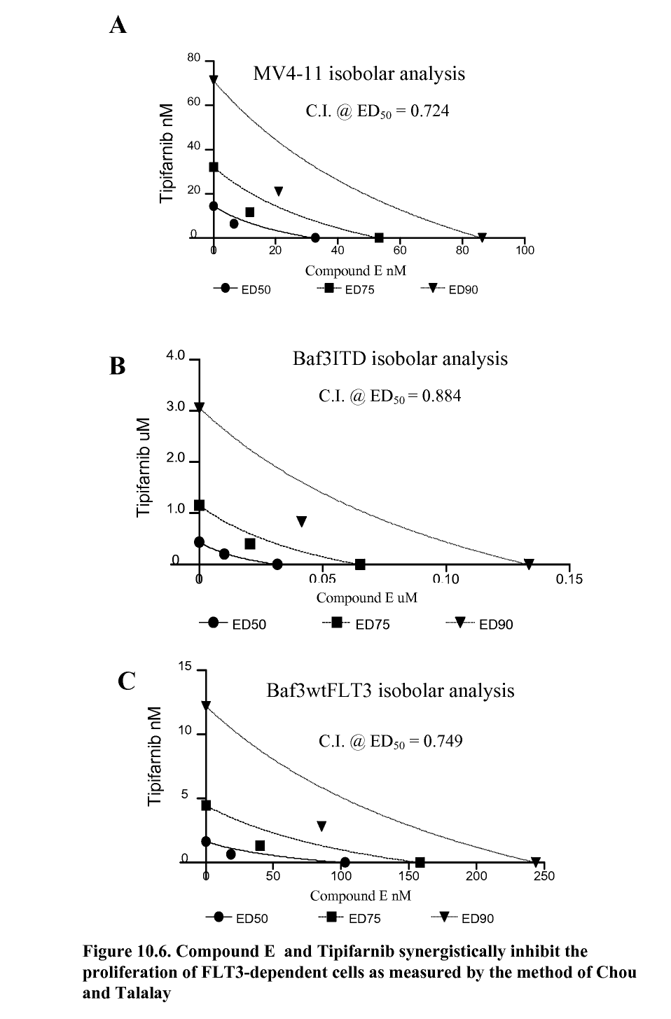

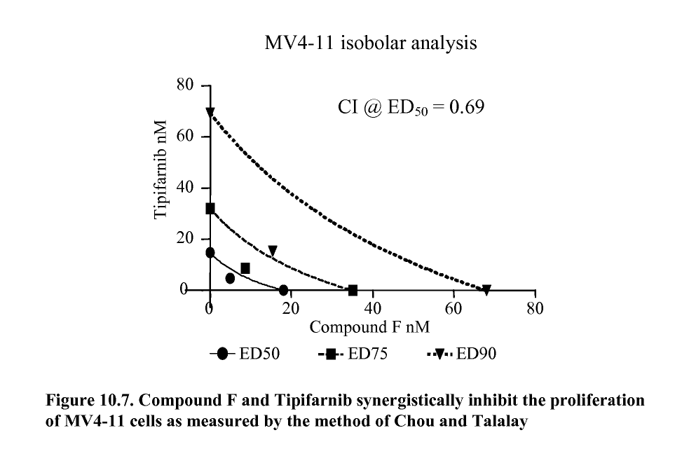

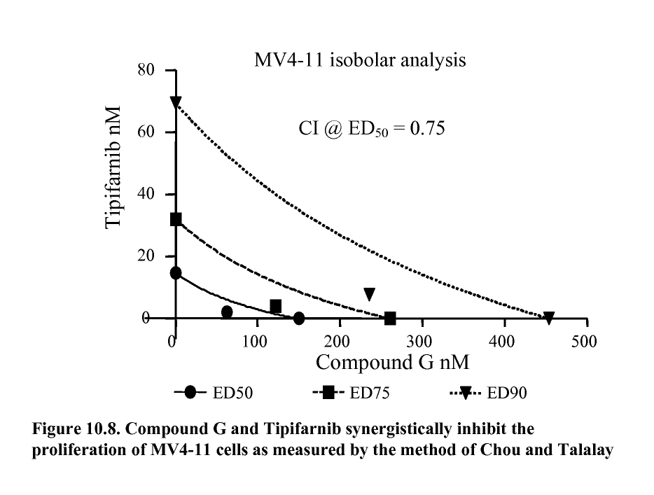

Dose responses for the individual compounds alone were determined for each cell line using a standard 72-hour cell proliferation assay (see FIGS. 6.1-6.8). The standard chemotherapeutic agent Cytarabine was used as a control cytotoxic agent in all experiments. The FTI Tipifarnib has a potency range of high nanomolar to high picomolar range depending on the cell type. The FLT3 inhibitors, Compounds A, B, C, D, E, F G and H, individually have good potency (sub-micromolar) for the inhibition of FLT3 driven proliferation (compared to the first line cytotoxic agent Cytarabine and Tipifamib) in cells that depend on FLT3 for growth. Each of these chemically distinct compounds alone has potential for the treatment of disorders related to FLT3, such as FLT3 positive AML. Cytarabine inhibition of proliferation is comparable (1-2 μM) to previous reports of its in vitro activity in MV4-11 cells (Levis, M., et al. (2004) “In vitro studies of a FLT3 inhibitor combined with chemotherapy: sequence of administration is important to achieve synergistic cytotoxic effects.” Blood. 104(4):1145-50). The FLT3 inhibitors tested had no effect on THP-1 proliferation. The IC₅₀ calculation for each compound in each cell line was used in subsequent combination experiments to calculate synergistic effects of compound combinations on cell proliferation. (See FIGS. 10.1-10.8 and Tables 1-3, hereafter.)

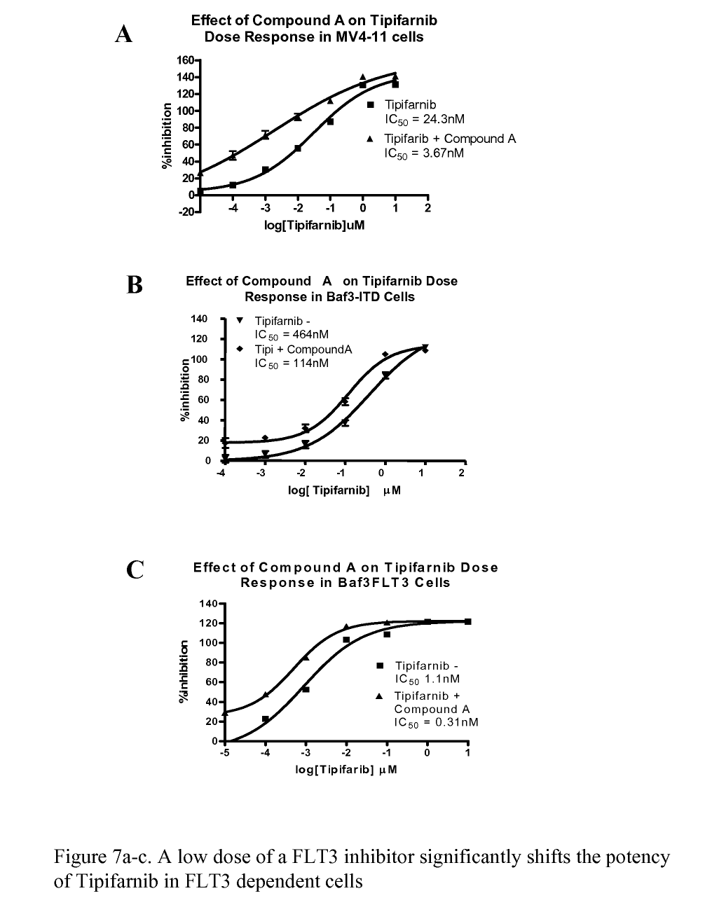

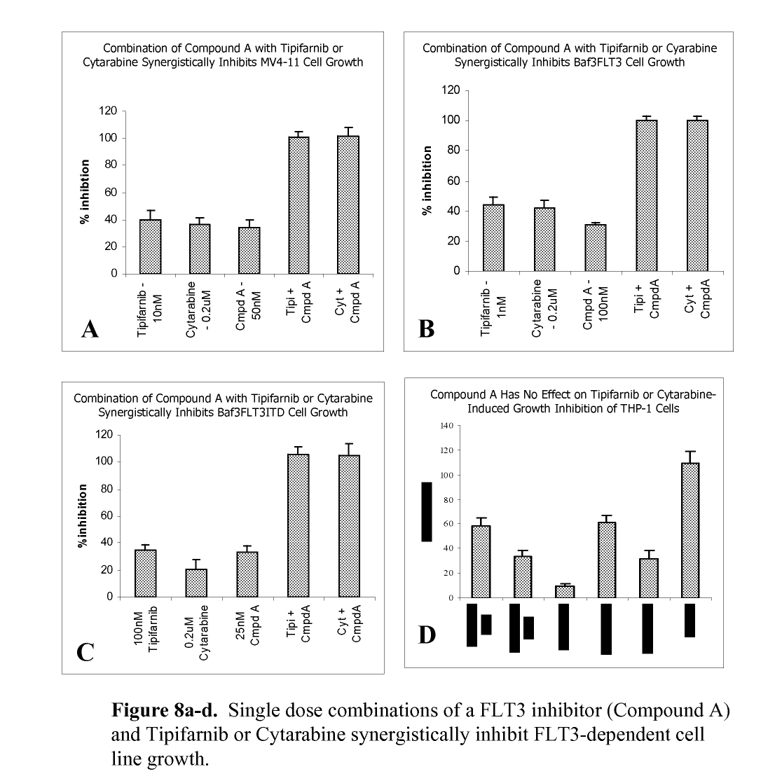

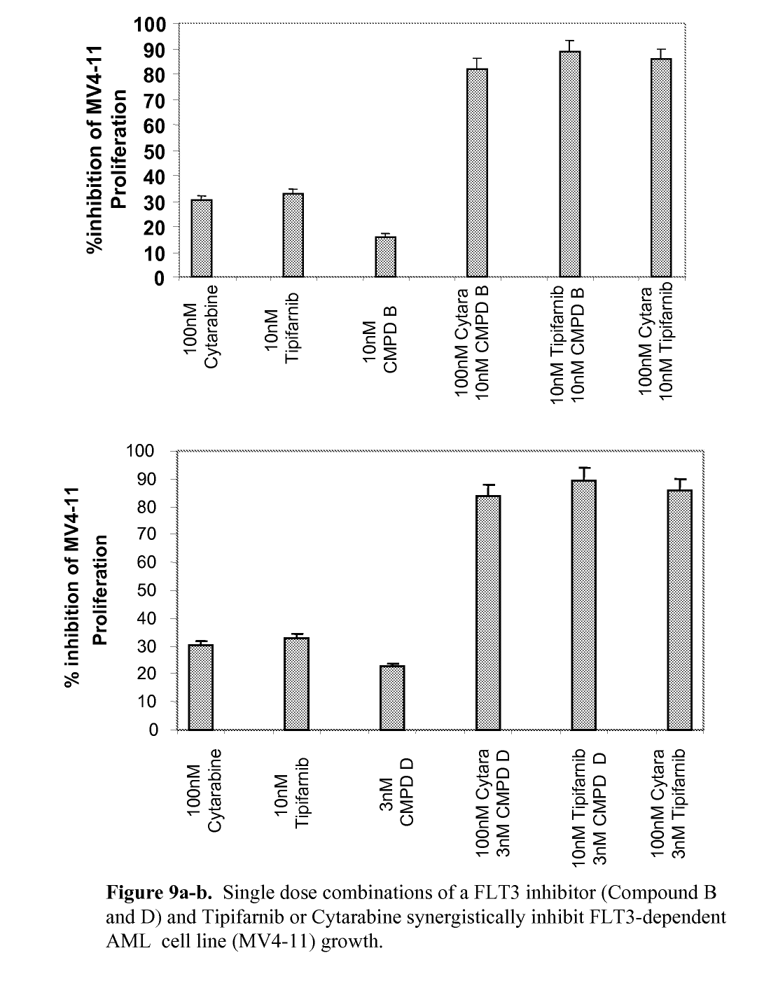

The effect of a single (sub-IC₅₀) dose of the FLT3 inhibitor Compound A on Tipifarnibpotency was then examined. Each cell line was simultaneously treated with one dose of the FLT3 inhibitor Compound A and varying doses of Tipifarnib and the proliferation of the cells was evaluated in the standard 72-hour cell proliferation protocol. The IC₅₀ for Tipifarnib was then calculated according to the procedure described in the Biological Activity section hereafter (see FIGS. 7a-c depicting results for FLT3 inhibitor Compound A and Tipifamib combination.) The cell lines that were tested included those that are dependent on FLT31TD mutant activity for growth (MV4-11 and Baf3-FLT31TD), FLT3 wt activity for growth (Baf3FLT3) and those that grow independent of FLT3 activity (THP-1).

The FLT3 inhibitor Compound A significantly increased the potency of the FTI Tipifarnib for the inhibition of AML (MV4-11) and FLT3 dependent (Baf3-ITD and Baf3-FLT3) cell proliferation. With a single sub-IC₅₀ dose of FLT3 inhibitor Compound A in (a) MV4-11 (50 nM); (b) BaB3-ITD (50 nM) and (c) BaB3-FLT3 (100 nM) cells, Tipifarnib increased in potency by more than 3-fold in each cell line tested. This is indicative of significant synergy.

Next, single dose combinations of the FTI Tipifarnib and the FLT3 inhbitor Compound A were evaluated in the MV4-11, BaB3-ITD and BaB3-FLT3 cell lines. This single dose combination scenario more closely represents dosing strategies for chemotherapeutic combinations that are used in the clinic. With this method cells are simultaneously treated with a single sub-IC₅₀ of dose of each compound or a combination of compounds and inhibition of proliferation was monitored. Using this method it is observed that combinations of a sub-IC₅₀ dose of the FTI Tipifamib and the FLT3 inhibitor Compound A are beyond additive in inhibiting the growth of the AML cell line MV4-11 and other FLT3-dependent cells (see FIGS. 8a-d). This synergistic effect with Tipifarnib is not observed in cells that do not depend on FLT3 for proliferation (THP-1). This synergistic effect was also observed for combinations of FLT3 inhibitor Compound A and Cytarabine.

Additionally, single dose combinations of a FLT3 inhibitor and a FTI were examined to determine if this activity was compound specific or mechanism based. A single sub-IC₅₀ of dose of either FLT3 inhibitor Compound B or D with Tipifarnib was tested for its inhibition of MV4-11 proliferation. It is observed, similar to combinations of Tipifarnib and FLT3 inhibitor Compound A, that the combinations of either FLT3 inhibitor Compound B or D with Tipifarnib inhibits the proliferation of FLT3-dependent MV4-11 cells with greater that additive efficacy. This suggests that the combination of any FLT3 inhibitor and FTI will synergistically inhibit the proliferation of FLT3-dependent AML cells. This observation is novel and non-obvious to those skilled in the art. Synergy was also observed with the combination of either FLT3 inhihbitor Compound B or D and cytarabine.

To statistically evaluate the synergy of a FLT3 inhibitor and an FTI in FLT3 dependent cell lines, dosing combinations were evaluated by the method of Chou and Talalay. See Chou T C, Talalay P. (1984) “Quantitative analysis of dose-effect relationships: the combined effects of multiple drugs or enzyme inhibitors.” Adv Enzyme Regul. 22:27-55. Using this method inhibitors are added simultaneously to cells in a ratio of the IC₅₀ dose of each compound alone. The data is collected and subject to isobolar analysis of fixed ratio dose combinations as described by Chou and Talalay. This analysis is used to generate a combination index or CI. The CI value of 1 corresponds to compounds that behave additively; CI values <0.9 are considered synergistic and CI values of >1.1 are considered antagonistic. Using this method, multiple FTI and FLT3 combinations were evaluated. For each experimental combination IC₅₀, were calculated for each individual compound (see FIGS. 6.1-6.8) in each of the FLT3 dependent cell lines and then fixed ratio dosing (at dose ranges including 9, 3, 1, 1/3, 1/9× the individual compound IC₅₀) was performed in the standard cell proliferation assay. FIGS. 10.1-10.8 summarizes the raw data from isobolar analysis fixed ratio dosing according to the method of Chou and Talalay, obtained using Calcusyn software (Biosoft). Using the isobologram, synergy can be graphically represented. Data points for combinations that are additive lie along the isobolar line at a given dose affect (CI=1). Data points for combinations that are synergistic fall to the left, or under, the isobolar line for a given dose effect (CI<0.9). Data points for combinations that are antagonistic fall to the right, or over, the isobolar line for a given dose effect (CI>1.1). FIG. 10.1a-c summarizes the isobolar analysis for the combination of FLT3 inhibitor Compound A and Tipifamib in MV4-11, Baf3-ITD and Baf3-wtFLT3. From the isobolar analysis, synergy was observed at all experimentally determined data points including the combination doses that resulted in a 50% inhibition of cell proliferation (ED50), a 75% inhibition of cell proliferation (ED75) and a 90% inhibition of cell proliferation (ED90). Each of these points falls significantly to the left of the isobolar (or additive) line, indicating significant synergy. The combination of FLT3 inhibitor Compound A and Tipifarnib resulted in significant synergy for proliferation inhibition in each FLT3 dependent cell lines tested. The combination indecies for the isobolograms depicted in FIGS. 10.1a-c are found in Tables 1-3 hereafter.

Additionally, FIGS. 10.2a-b summarizes the isobolar analysis with the combination of a chemically distinct FLT3 inhibitor, FLT3 inhibitor Compound B and Tipifarnib. Similar to the FLT3 inhibitor Compound A and Tipifamib combination, the FLT3 inhibitor Compound H and Tipifamib combination was synergistic for inhibiting cellular proliferation at all doses tested and in all FLT3-dependent cell lines tested. The combination indecies for the isobolargrams depicted in FIGS. 5.2a-c are found in Tables 1-3 hereafter. Futhermore, FIGS. 5.3a-c summarizes the isobolar analysis of a combination of Tipifamib and another chemically distinct FLT3 inhibitor (FLT3 inhibitor Compound E). As with the other combinations tested, the combination of FLT3 inhibitor compound E and Tipifarnib synergistically inhibited FLT3-dependent proliferation in three different cell lines at all doses tested. The combination indecies for the isobolargrams depicted in FIGS. 5.3a-c are found in Tables 1-3 hereafter.

To further expand the combination studies, each of the FLT3 inhibitors shown to demonstrate synergy with Tipifarnib were also tested in combination with another farnesyl transferase inhibitor, FTI-176. Tables 1-3 summarize the results of all the combinations tested in the three FLT3-dependent cell lines described above. The combination indecies for each combination are contained within Tables 1-3.

TABLE 1
Table 1: The combination of a FLT3 inhibitor and an FTI (all combinations tested)
synergistically inhibits the proliferation of MV4-11 AML cells as measured by the
Combination Index (CI). Combinations were performed at a fixed ratio of the
individual compound IC₅₀ₛ for proliferation as summarized in Biological Activity
Measurments section hereafter. IC₅₀ and CI values were calculated by the method of
Chou and Talalay using Calcusyn software (Biosoft). CI and IC₅₀ values are an
average of three independent experiments with three replicates per data point.

| | | | | FTI | |
|---|---|---|---|---|---|
| | | | | IC50 | FLT3 inhibitor IC50 |
| MV4-11 cells | CI-ED50 | CI-ED75 | CI-ED90 | (nM) | (nM) |
| Tipifarnib | | | | 15.41 | |
| FTI-176 | | | | 17.73 | |
| FLT3 inhibitor Compound A | | | | | 92.53 |
| FLT3 inhibitor Compound B | | | | | 31.3 |
| FLT3 inhibitor Compound C | | | | | 18.1 |
| FLT3 inhibitor Compound D | | | | | 13.8 |
| FLT3 inhibitor Compound H | | | | | 166.93 |
| FLT3 inhibitor Compound E | | | | | 32.81 |
| Tipifarnib + FLT3 | 0.58 | 0.52 | 0.46 | 3.96 | 28.12 |
| inhibitor Compound A | | | | | |
| Tipifarnib + FLT3 | 0.79 | 0.66 | 0.60 | 4.48 | 9.86 |
| inhibitor Compound B | | | | | |
| Tipifarnib + FLT3 | 0.78 | 0.62 | 0.55 | 3.65 | 3.86 |
| inhibitor Compound C | | | | | |
| Tipifarnib + FLT3 | 0.67 | 0.62 | 0.59 | 4.19 | 3.75 |
| inhibitor Compound D | | | | | |
| Tipifarnib + FLT3 | 0.56 | 0.51 | 0.48 | 4.39 | 64.81 |
| inhibitor Compound H | | | | | |
| Tipifarnib + FLT3 | 0.67 | 0.62 | 0.59 | 4.19 | 1.75 |
| inhibitor Compound E | | | | | |
| Tipifarnib + FLT3 | 0.69 | 0.59 | 0.55 | 4.23 | 11.67 |
| inhibitor Compound F | | | | | |
| Tipifarnib + FLT3 | 0.75 | 0.61 | 0.68 | 4.84 | 145.15 |
| inhibitor Compound G | | | | | |
| FTI 176 + FLT3 | 0.62 | 0.60 | 0.59 | 4.63 | 30.12 |
| inhibitor Compound A | | | | | |
| FTI 176 + FLT3 | 0.66 | 0.63 | 0.61 | 5.81 | 50.94 |
| inhibitor Compound H | | | | | |
| FTI 176 + FLT3 | 0.68 | 0.64 | 0.61 | 5.69 | 9.37 |
| inhibitor Compound E | | | | | |
| FTI 176 + FLT3 | 0.71 | 0.63 | 0.60 | 4.72 | 5.48 |
| inhibitor Compound D | | | | | |

TABLE 2
Table 2: The combination of a FLT3 inhibitor and an FTI (all combinations tested)
synergistically inhibits the proliferation of Baf3-FLT3 cells stimulated with 100 ng/ml
FLT ligand as measured by the Combination Index (CI). Combinations were
performed at a fixed ratio of the individual compound IC50s for proliferation as
summarized in Biological Activity Measurments section hereafter. IC50 and CI
values were calculated by the method of Chou and Talalay using Calcusyn software
(Biosoft). CI and IC₅₀ values are an average of three independent experiments with
three replicates per data point.

| | | | | FTI | FLT3 inhibitor |
|---|---|---|---|---|---|
| Baf3-FLT3 | CI-ED50 | CI-ED75 | CI-ED90 | IC50 (nM) | IC50 (nM) |
| Tipifarnib | | | | 1.85 | |
| FTI-176 | | | | 1.35 | |
| FLT3 inhibitor Compound A | | | | | 169.77 |
| FLT3 inhibitor Compound B | | | | | 173.1 |
| FLT3 inhibitor Compound C | | | | | 91.3 |
| FLT3 inhibitor Compound D | | | | | 39.90 |
| FLT3 inhibitor Compound H | | | | | 451.37 |
| FLT3 inhibitor Compound E | | | | | 29.40 |
| Tipifarnib + FLT3 | 0.45 | 0.40 | 0.37 | 0.333 | 48.24 |
| inhibitor Compound A | | | | | |
| Tipifarnib + FLT3 | 0.78 | 0.67 | 0.62 | 0.431 | 23.26 |
| inhibitor Compound B | | | | | |
| Tipifarnib + FLT3 | 0.81 | 0.71 | 0.65 | 0.442 | 63.41 |
| inhibitor Compound C | | | | | |
| Tipifarnib + FLT3 | 0.60 | 0.53 | 0.49 | 0.360 | 12.31 |
| inhibitor Compound D | | | | | |
| Tipifarnib + FLT3 | 0.38 | 0.36 | 0.35 | 0.277 | 125.28 |
| inhibitor Compound H | | | | | |
| Tipifarnib + FLT3 | 0.42 | 0.39 | 0.38 | 0.360 | 23.26 |
| inhibitor Compound E | | | | | |
| FTI 176 + FLT3 | 0.55 | 0.40 | 0.32 | 0.374 | 56.33 |
| inhibitor Compound A | | | | | |
| FTI 176 + FLT3 | 0.60 | 0.56 | 0.48 | 0.380 | 11.61 |
| inhibitor Compound D | | | | | |
| FTI 176 + FLT3 | 0.44 | 0.34 | 0.27 | 0.290 | 145.11 |
| inhibitor Compound H | | | | | |
| FTI 176 + FLT3 | 0.49 | 0.39 | 0.33 | 0.391 | 25.16 |
| inhibitor Compound E | | | | | |

TABLE 3
Table 3: The combination of a FLT3 inhibitor and an FTI (all combinations tested)
synergistically inhibits the proliferation of Baf3-ITD cells as measured by the
Combination Index (CI). Combinations were performed at a fixed ratio of the
individual compound IC50s for proliferation as summarized in Biological Activity
Measurments section hereafter. IC50 and CI values were calculated by the method of
Chou and Talalay using Calcusyn software (Biosoft). CI and IC₅₀ values are an
average of three independent experiments with three replicates per data point.

| | | | | | FLT3 inhibitor |
|---|---|---|---|---|---|
| Baf3-FLT3 cells | CI-ED50 | CI-ED75 | CI-ED90 | FTI IC50 (nM) | IC50 (nM) |
| Tipifarnib | | | | 547.87 | |
| FTI-176 | | | | 667.86 | |
| FLT3 inhibitor | | | | | 76.12 |
| Compound A | | | | | |
| FLT3 inhibitor | | | | | 14.56 |
| Compound D | | | | | |
| FLT3 inhibitor | | | | | 200.17 |
| Compound H | | | | | |
| FLT3 inhibitor | | | | | 29.40 |
| Compound E | | | | | |
| Tipifarnib + FLT3 | 0.72 | 0.63 | 0.62 | 146.83 | 27.19 |
| inhibitor | | | | | |
| Compound A | | | | | |
| Tipifarnib + FLT3 | 0.68 | 0.65 | 0.63 | 165.60 | 4.87 |
| inhibitor | | | | | |
| Compound D | | | | | |
| Tipifarnib + FLT3 | 0.92 | 0.87 | 0.84 | 172.80 | 71.49 |
| inhibitor | | | | | |
| Compound H | | | | | |
| Tipifarnib + FLT3 | 0.82 | 0.78 | 0.75 | 189.10 | 11.85 |
| inhibitor | | | | | |
| Compound E | | | | | |
| FTI 176 + FLT3 | 0.74 | 0.62 | 051 | 224.36 | 25.37 |
| inhibitor | | | | | |
| Compound A | | | | | |
| FTI 176 + FLT3 | 0.75 | 0.69 | 0.63 | 231.68 | 4.12 |
| inhibitor | | | | | |
| Compound D | | | | | |
| FTI 176 + FLT3 | 0.62 | 0.60 | 0.58 | 183.38 | 68.54 |
| inhibitor | | | | | |
| Compound H | | | | | |
| FTI 176 + FLT3 | 0.51 | 0.50 | 0.50 | 220.80 | 8.91 |
| inhibitor | | | | | |
| Compound E | | | | | |

Synergy of combination dosing is observed with all FTI and FLT3 combinations tested in all FLT3 dependent cell lines used. The combination of an FTI and FLT3 inhibitor reduces the individual compounds antiproliferative effect by an average of 3-4 fold. It can be concluded that the synergy observed for combinations of a FLT3 inhibitor and an FTI is a mechanism based phenomena and not related to the specific chemical structures of individual FTIs or FLT3 inhibitors. Accordingly, synergistic growth inhibition would be observed with any combination of a FLT3 inhibitor and Tipifarnib or any other FTI.

The ultimate goal of treatment for FLT3 related disorders is to kill the disease causative cells and to cause regression of disease. To examine if the FTI/FLT3 inhibitor combination is synergistic for cell death of FLT3 dependent disease causative cells, particularly AML, ALL and MDS cells, the combination of Tipifarnib and the FLT3 inhibitor Compound A was tested for its ability to induce an increase in fluorescent labeled Annexin V staining in MV4-11 cells. Annexin V binding to phosphotidyl serine that has translocated from the inner leaflet of the plasma membrane to the outer leaflet of the plasma membrane and is a well established way to measure apoptosis of cells. See van Engeland M., L. J. Nieland, et al. (1998) “Annexin V-affinity assay: a review on an apoptosis detection system based on phosphatidylserine exposure.” Cytometry. 31(1):1-9.

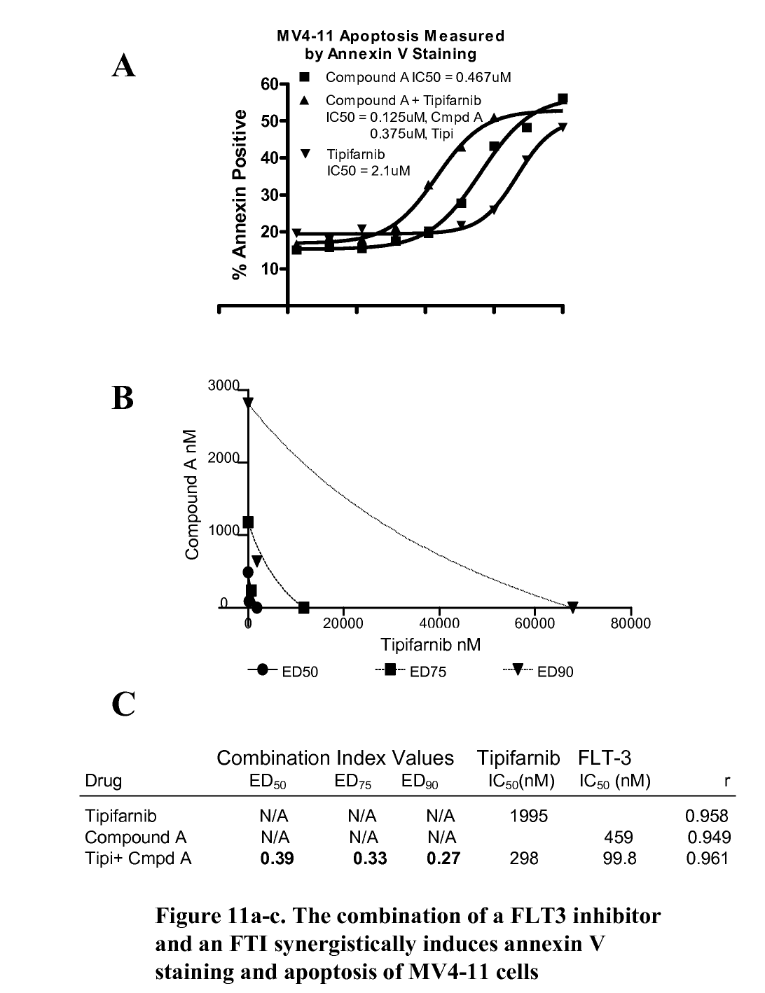

Tipifarnib and FLT3 inhibitor Compound A were incubated with MV4-11 cells alone or in a fixed ratio (4:1 based on the calculated EC₅₀ for each agent alone) for 48 hours in standard cell culture conditions. After the compound incubations, treated cells were harvested and stained with Annexin V-PE and 7-AAD using the Guava Nexin apoptosis kit according to the protocol in the Biological Activity Measurements section hereafter. Annexin V staining peaks at 60% because cells late in apoptosis begin to fall apart and are considered debris. However, EC₅₀, can be calculated from this data because of its consistent sigmoidal kinetics. From the data summarized in FIG. 11a, it is concluded that the combination of Tipifarnib and FLT3 inhibitor Compound A is significantly more potent than either agent alone for inducing apoptosis of MV4-11 cells. The EC₅₀ for the induction of annexin V staining shifted more than 4-fold for the FLT3 inhibitor FLT3 inhibitor Compound A. The EC₅₀ for induction of annexin V staining shifted by more than eight-fold for the FTI Tipifarnib. Statistical analysis using the above described method of Chou and Talalay was also performed to determine the synergy of the combination. FIG. 11b depictes the isobolar analysis of the Tipifarnib and FLT3 inhibitor Compound A combination in inducing annexin V staining. All data points lie significantly to the left of the isobolar line. The CI values for the combination are listed in the table in FIG. 11c. The synergy that was observed for annexin V staining (and induction of apoptosis) were more significant than the synergies that were observed for the FLT3 inhibitor and FTI combinations for proliferation. The magnitude of the synergistic induction of apoptosis of MV4-11 cells by the combination of an FTI and a FLT3 inhibitor could not be predicted by those skilled in the art. Thus, based on the data from proliferation, any combination of a FLT3 inhibitor and an FTI would also be synergistic for inducing apoptosis of FLT3 dependent cells (i.e. causative cells for FLT3 disorders, particularly AML, ALL and MDS).

To confirm that the combination of a FLT3 inhibitor and an FTI synergistically activates apoptosis of FLT3 dependent cells, the combination of several FLT3 inhibitors and the FTI Tipifarnib was tested for its ability to induce the activity of caspase 3/7 in MV4-11 cells. Caspase activation, a critical step in the final execution of the apoptotic cellular death process, can be induced by a variety of cellular stimuli including growth factor withdrawal or growth factor receptor inhibition See Hengartner, M O. (2000) “The biochemistry of apoptosis.” Nature 407:770-76 and Nunez G, Benedict M A, Hu Y, Inohara N. (1998) “Caspases: the proteases of the apoptotic pathway.” Oncogene 17:3237-45. Cellular caspase activation can be monitored using a synthetic caspase 3/7 substrate that is cleaved to release a substrate for the enzyme luciferase, that may convert the substrate to a luminescent product. See Lovborg H, Gullbo J, Larsson R. (2005) “Screening for apoptosis-classical and emerging techniques.” Anticancer Drugs 16:593-9. Caspase activation was monitored using the Caspase Glo technology from Promega (Madison, Wis.) according to the protocol in the Biological Activity Measurement section hereafter.

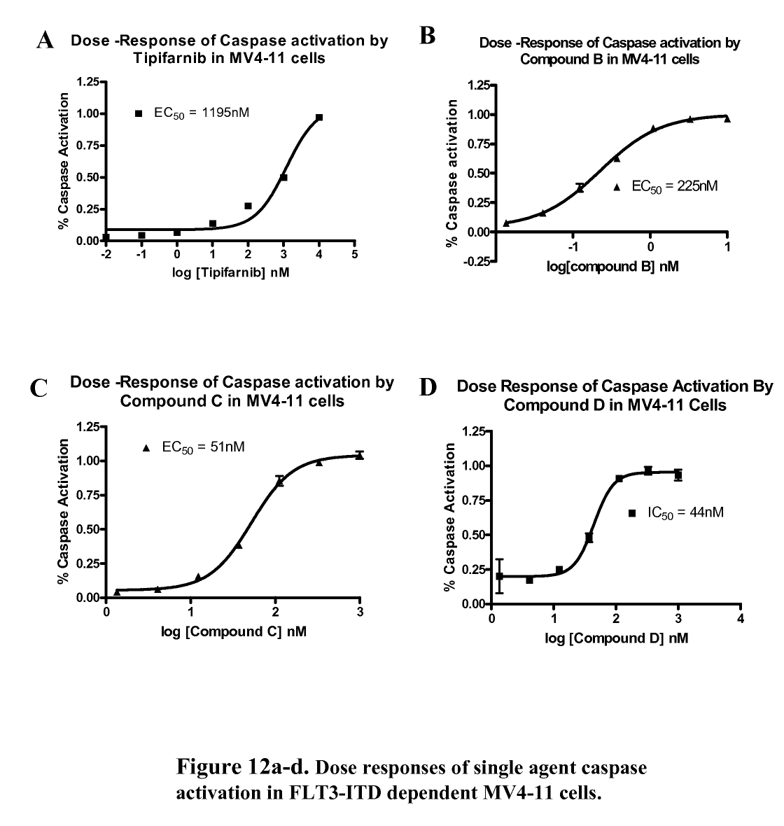

Individual EC₅₀ determinations were done to establish dose ratios for combination analysis of synergy. FIG. 12a-d summarizes the EC₅₀ determinations of each individual agent. For combination experiments, Tipifarnib and FLT3 inhibitor Compounds B, C and D were incubated with MV4-11 cells in a fixed ratio (based on the calculated EC₅₀ for each agent alone) at various doses (ranges including 9, 3, 1, 1/3, 1/9× the individual compound EC₅₀) for 24 hours in standard cell culture conditions. After 24 hours the caspase 3/7 activity was measured according to the manufacture's instructions and detailed in the Biological Activity Measurement section hereafter.

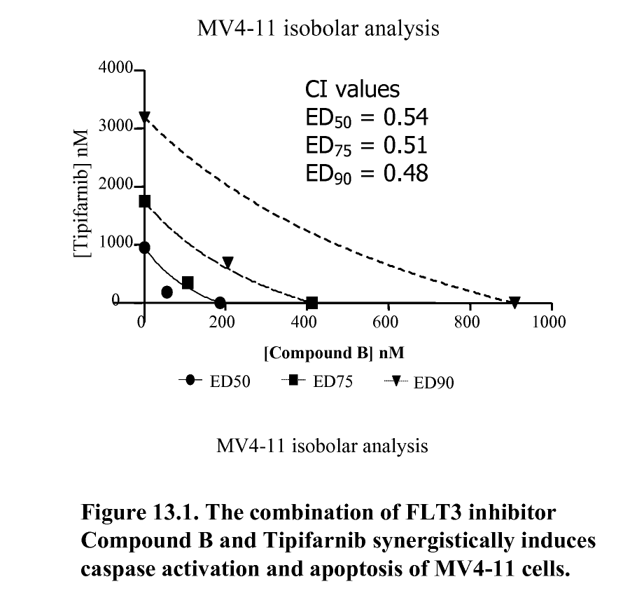

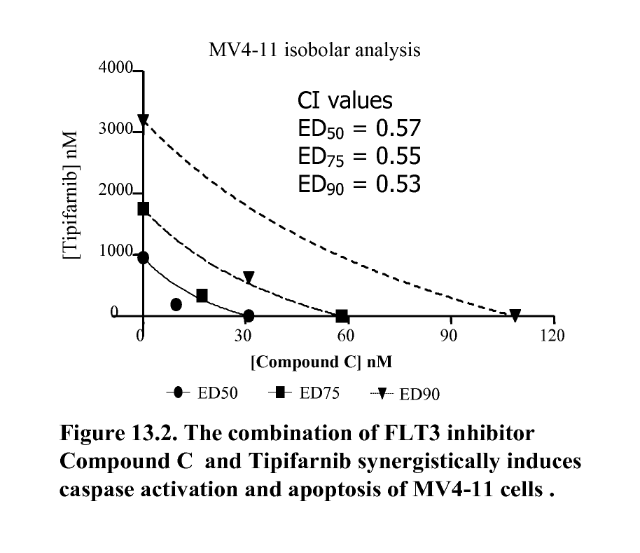

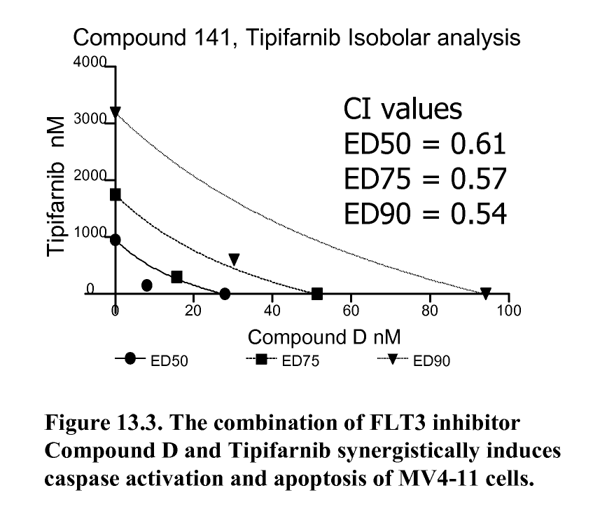

FIG. 13.1-13.3 summarizes the synergy of caspase activation (by the method previously described method of Chou and Talalay) that was observed with the Tipifarnib and FLT3 inhibitor Compounds B, C and D combinations in MV4-11 cells. Synergy was observed at all doses tested and in all combinations tested. The synergy that was observed for caspase activation (and induction of apoptosis) was even more significant than the synergies that were observed for the FLT3 inhibitor and FTI combinations for proliferation in MV4-11 cells. The magnitude of the synergistic induction of apoptosis of MV4-11 cells by the combination of an FTI and a FLT3 inhibitor could not be predicted by those skilled in the art. Thus, based on the data from proliferation, any combination of a FLT3 Inhibitor and an FTI would also be synergistic for inducing apoptosis of FLT3 dependent cells (i.e. causative cells for FLT3 disorders, particularly AML, ALL and MDS).

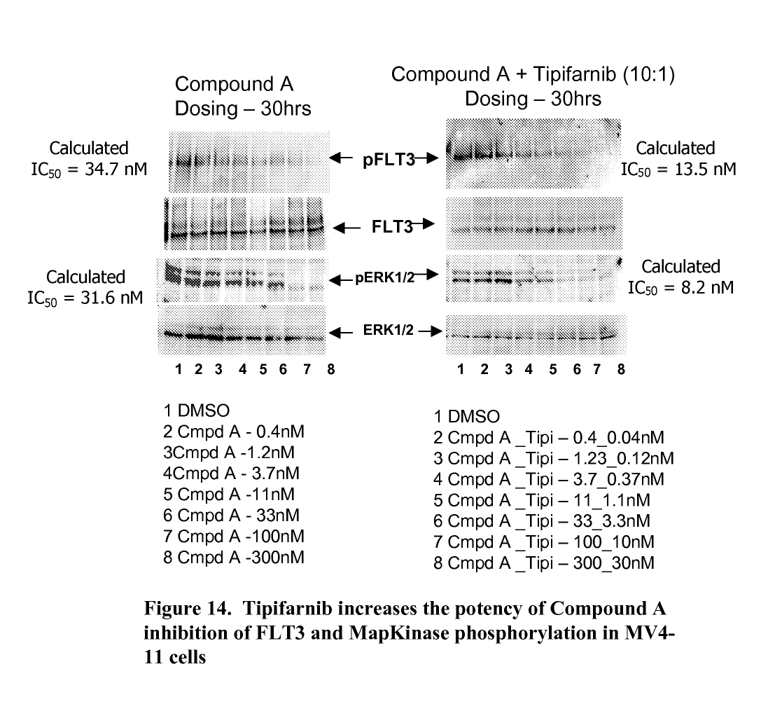

It is well established that phosphorylation of the FLT3 receptor and downstream kinases such as MAP kinase are required for proliferative effects of FLT3 receptor. See Scheijen, B. and J. D. Griffin (2002) “Tyrosine kinase oncogenes in normal hematopoiesis and hematological disease.” Oncogene 21(21): 3314-33. We postulate that the molecular mechanism of the synergy observed with a FLT3 inhibitor and an FTI is related to the compound induced decrease of FLT3 receptor signaling required for AML cell proliferation and survival. To test this we looked at phosphorylation state of both the FLT3-ITD receptor and a downstream target of FLT3 receptor activity, MAP kinase (erk1/2) phosphorylation in MV4-11 cells, using commercially available reagents according to the protocol detailed in the Biological Activity Measurements section hereafter. MV4-11 cells were treated with indicated concentrations of FLT3 inhibitor Compoud A alone or in combination with Tipifarnib for 48 hours under standard cell growth conditions. For analysis of FLT3 phosphorylation, cells were harvested and FLT3 was immunoprecipitated and separated by SDS-PAGE. For analysis of MAP kinase (erk1/2) phosphorylation, cells were harvested, subjected to lysis, separated by SDS-Page and transferred to nitrocellulose for immunoblot analysis. For quantitative analysis of FLT3 phosphorylation, immunoblots were probed with phosphotyrosine antibody and the phophoFLT3 signal was quantified using Molecular Dynamics Typhoon Image Analysis. The immunoblots were then stripped and reprobed to quantify the total FLT3 protein signal. This ratio of phosphorylation to total protein signal was used to calculate the approximate IC₅₀ of the compound dose responses. For quantitative analysis of MAP kinase (ERK1/2) phosphorylation, immunoblots were probed with a phosphospecific ERK1/2 antibody and the phophoERK1/2 signal was quantified using Molecular Dynamics Typhoon Image Analysis. The immunoblots were then stripped and reprobed to quantify the total ERK1/2 protein signal. This ratio of phosphorylation to total protein signal was used to calculate the approximate IC₅₀ of the compound dose responses. IC₅₀ values were calculated using GraphPad Prism software. The result of this work is summarized in FIG. 14.

It is observed that the combination of Tipifamib and FLT3 inhibitor Compound A increases the potency of FLT3 inhibitor Compound A two to three fold for both inhibition of FLT3 phosphorylation and MAP kinase phosphorylation. This is consistent with the increase in potency of the compounds anti-proliferative effects. The effect of FLT3 phosphorylation that was observed with the FTI/FLT3 inihbitor combination has not been reported previously. The mechanism for this effect on FLT3 phosphorylation is unknown but would be predicted to occur for any FTI/FLT3 inhibitor combination based on the experimental data collected for proliferation inhibition described above.

In Vitro Biological Activity Measurements

Reagents and Antibodies. Cell Titerglo proliferation reagent was obtained from Promega Corporation. Proteases inhibitor cocktails and phosphatase inhibitor cocktails II were purchased from Sigma (St. Louis, Mo.). The GuavaNexin apoptosis reagent was purchased from Guava technologies (Hayward, Calif.). Superblock buffer and SuperSignal Pico reagent were purchased from Pierce Biotechnology (Rockford, Ill.). Fluorescence polarization tyrosine kinase kit (Green) was obtained from Invitrogen. Mouse anti-phosphotyrosine (4G10) antibody was purchased from Upstate Biotechnology, Inc (Charlottesville, Va.). Anti-human FLT3 (rabbit IgG) was purchased from Santa Cruz biotechnology (Santa Cruz, Calif.). Anti-phospho Map kinase and total p42/44 Map kinase antibodies were purchased form Cell Signaling Technologies (Beverly, Mass.) Alkaline phosphatase-conjugated goat-anti-rabbit IgG, and goat-anti-mouse IgG antibody purchased from Novagen (San Diego, Calif.). DDAO phosphate was purchased from Molecular Probes (Eugene, Oreg.). All tissue culture reagents were purchase from BioWhitaker (Walkersville, Md.).

Cell lines. THP-1 (Ras mutated, FLT3 wild type) and human MV4-11 (expressing constitutively FLT3-Internal tandem duplication or ITD mutant isolated from an AML patient with a t15;17 translocation) AML cells)(see Drexler H G. The Leukemia-Lymphoma Cell Line Factsbook. Academic Pres: San Diego, Calif., 2000 and Quentmeier H, Reinhardt J, Zaborski M, Drexler H G. FLT3 mutations in acute myeloid leukemia cell lines. Leukemia. 2003 January; 17:120-124.) were obtained from ATCC (Rockville, Md.). The IL-3 dependent murine B-cell progenitor cell line Baf3 expressing human wild-type FLT3 (Baf3-FLT3) and ITD-mutated FLT3 (Baf3-ITD) were obtained from Dr. Michael Heinrich (Oregon Health Sciences University). Cells were maintained in RPMI media containing penn/strep, 10% FBS alone (THP-1, Baf3-ITD) and 2 ng/ml GM-CSF (MV4-11) or 10 ng/ml FLT ligand (Baf3-FLT3). MV4-11, Baf3-ITD and Baf3-FLT3 cells are all absolutely dependent on FLT3 activity for growth. GM-CSF enhances the activity of the FLT3-ITD receptor in the MV4-11 cells.

Cell proliferation assay for MV4-11, Baf3-ITD, Baf3-FLT3 and THP-1 cells. To measure proliferation inhibition by test compounds the luciferase based CellTiterGlo reagent (Promega) was used. Cells are plated at 10,000 cells per well in 100 ul of in RPMI media containing penn/strep, 10% FBS alone (THP-1, Baf3-ITD) and 0.2 ng/ml GM-CSF (MV4-11) or 10 ng/ml FLT ligand (Baf3-FLT3). Compound dilutions or 0.1% DMSO (vehicle control) are added to cells and the cells are allowed to grow for 72 hours at standard cell growth conditions (37° C., 5% CO₂). In combination experiments test agents were added simultaneously to the cells. Total cell growth is quantified as the difference in luminescent counts (relative light units, RLU) of cell number at Day 0 compared to total cell number at Day 3 (72 hours of growth and/or compound treatment). One hundred percent inhibition of growth is defined as an RLU equivalent to the Day 0 reading. Zero percent inhibition is defined as the RLU signal for the DMSO vehicle control at Day 3 of growth. All data points are an average of triplicate samples. The IC₅₀ for growth inhibition represents the dose of a compound that results in a 50% inhibition of total cell growth at Day 3 of the DMSO vehicle control. IC₅₀ data analysis was done with GraphPad Prism using a non-linear regression fit with a multiparameter, sigmoidal dose-response (variable slope) equation.

Immunoprecipitation and Quantitative Immunoblot Analysis. MV4-11 cells were grown in DMEM supplemented with 10% fetal bovine serum, 2 ng/ml GM-CSF and kept between 1×10⁵ and 1×10⁶ cells/ml. For western blot analysis of Map Kinase phosphorylation 1×10⁶ MV4-11 cells per condition were used. For immunoprecipitation experiments examining FLT3-ITD phosphorylation, 1×10⁷ cells were used for each experimental condition. After compound treatment, MV4-11 cells were washed once with cold 1×PBS and lysed with HNTG lysis buffer (50 mM Hepes, 150 mM NaCl, 10% Glycerol, 1% Triton-X-100, 10 mM NaF, 1 mM EDTA, 1.5 mM MgCl2, 10 mM NaPyrophosphate)+4 ul/ml Protease Inhibitor Cocktail (Sigma cat.#P8340)+4 ul/ml Phosphatase Inhibitor Cocktail (Sigma Cat#P2850). Nuclei and debris were removed from cell lysates by centrifugation (5000 rpm for 5 min. at 4° C.). Cell lysates for immunoprecipitation were cleared with agarose-Protein A/G for 30 minutes at 4° C. and immunoprecipitated using the 3 ug of FLT3 antibody for 1 hours at 4° C. Immune complexes were then incubated with agarose-Protein A/G for 1 hour at 4° C. Protein A/G immunoprecipitates were washed three times in 1.0 ml of HNTG lysis buffer. Immunoprecipitates and cell lysates (40 ug total protein) were resolved on a 10% SDS-PAGE gel, and the proteins were transferred to nitrocellulose membrane. For anti-phosphotyrosine immunoblot analysis, membranes were blocked with SuperBlock (Pierce) and blotted for 2 hours with anti-phosphotyrosine (clone 4G10, Upstate Biotechnologies) followed by alkaline phosphatase-conjugated goat anti-mouse antibody. For anti-phosphoMAP kinase western blotting, membranes were blocked Super block for 1 hour and blotted overnight in primary antibody, followed by an incubation with an AP conjugated goat-anti rabbit secondary antibody. Detection of protein was done by measuring the fluorescent product of the alkaline phosphatase reaction with the substrate 9H-(1,3-dichloro-9,9-dimethylacridin-2-one-7-yl)phosphate, diammonium salt (DDAO phosphate) (Molecular Probes) using a Molecular Dynamics Typhoon Imaging system (Molecular Dynamics, Sunyvale, Calif.). Blots were stripped and reprobed with anti-FLT3 antibody for normalization of phosphorylation signals. Quantitation of DDAO phosphate signal and IC₅₀ determinations were done with Molecular Dynamics ImageQuant and GraphPad Prism software.

Annexin V Staining. To examine the apoptosis of the leukemic MV4-11 cell line, cells were treated with Tipifarnib and/or FLT3 inhibitor Compound A, and Annexin V binding to phosphotidylserine on the outer leaflet of the plasma membrane of apoptotic cells was monitored using the GuavaNexin assay reagent and the Guava personal flow cytometry system (Guava Technologies; Hayward, Calif.). MV4-11 cells were plated at 200,000 cells per ml in tissue culture media containing varying concentrations of Tipifarnib and/or FLT3 inhibitor Compound A and incubated for 48 hours at 37° C., 5% CO₂. Cells were harvested by centrifugation at 400×g for 10 minutes at 4° C. Cells were then washed with 1×PBS and resuspended in 1× Nexin buffer at 1×10⁶ cells/ml. 5 μl of Annexin V-PE ad 5 μl of 7-AAD was added to 40 μl of cell suspension and incubated on ice for 20 minutes protected from light. 450 ml of cold 1× Nexin buffer was added to each sample and the cells were then acquired on the Guava cytometer according to the manufacturer's instructions. All annexin positive cells were considered apoptotic and percent Annexin positive cells was calculated.

Caspase 3/7 Activation Assay. MV4-11 cells were grown in RPMI media containing pen/strep, 10% FBS and 1 ng/mL GM-CSF. Cells were maintained between 2×10⁵ cells/mL and 8×10⁵ cells/mL feeding/splitting every 2-3 days. Cells were centrifuged and resuspend at 2×10⁵ cells/mL RPMI media containing Penn/Strep, 10% FBS and 0.1 ng/mL GM-CSF. MV4-11 cells were plated at 20,000 cells per well in 100 μL of in RPMI media containing penn/strep, 10% FBS alone and 0.1 ng/mL GM-CSF (Corning Costar Cat # 3610) in the presence of various concentrations of test compounds or DMSO. In combination experiments test agents were added simultaneously to the cells. Cells were incubated for 24 hours at 37° C., 5% CO₂. After 24-hour incubation, caspase activity was measured with the Promega CaspaseGlo reagent (Cat# G8090) according to the manufacture's instructions. Briefly, CaspaseGlo substrate is diluted with 10 mL Caspase Glo buffer. One volume of diluted Caspase Glo reagent was added to one volume of tissue culture media and mixed for two minutes on rotating orbital shaker. Following incubation at room temperature for 60 minutes, light emission was measured on a Berthold luminometer with the 1 second program. Baseline caspase activity was defined as an RLU equivalent to DMSO vehicle (0.1% DMSO) treated cells. EC₅₀ data analysis was completed with GraphPad Prism using a non-linear regression fit with a multiparameter, sigmoidal dose-response (variable slope) equation.

Combination Index Analysis. To determine growth inhibition synergy of a FTI and FLT3 inhibitor combination based on the method of Chou and Talalay (Chou and Talalay. See Chou T C, Talalay P. (1984) “Quantitative analysis of dose-effect relationships: the combined effects of multiple drugs or enzyme inhibitors.” Adv Enzyme Regul. 22:27-55.), fixed ratio combination dosing with isobolar statistical analysis was performed. Test agents were combined at a fixed ratio of the individual IC₅₀ for proliferation for each cell line and dosed at varying concentrations including 9, 3, 1, 1/3, 1/9 times the determined IC₅₀ dose. To measure proliferation inhibition by test combinations the luciferase based CellTiterGlo reagent (Promega) was used. Cells are plated at 10,000 cells per well in 100 ul of in RPMI media containing penn/strep, 10% FBS alone (THP-1, Baf3-ITD) and 0.1 ng/ml GM-CSF (MV4-11) or 100 ng/ml FLT ligand (Baf3-FLT3). Total cell growth is quantified as the difference in luminescent counts (relative light units, RLU) of cell number at Day 0 compared to total cell number at Day 3 (72 hours of growth and/or compound treatment). All data points are an average of triplicate samples. One hundred percent inhibition of growth is defined as an RLU equivalent to the Day 0 reading. Zero percent inhibition is defined as the RLU signal for the DMSO vehicle control at Day 3 of growth. Inhibition data was analyzed using Calcsyn (BioSoft, Ferguson, Mo.) and the combination index (C.I.) calculated. C.I. values <0.9 are considered synergistic.

In Vivo Combination Studies

The effect of combination treatment of the FLT3 Inhibitor FLT3 inhibitor compounds and Tipifarnib (Zarnestra™) on the growth of MV-4-11 human AML tumor xenografts in nude mice was tested using FLT3 inhibitor Compounds B and D. The in vivo study was designed to extend the in vitro observations to evaluate the potential for a synergistic anti-tumor effect of FLT3 inhibitor Compounds B and D each administered orally together with Tipifarnib to nude mice bearing established MV-4-11 tumor xenografts.

Anti-Tumor Effect of FLT3 Inhibitor Compound B Alone

Female athymic nude mice (CD-1, nu/nu, 9-10 weeks old) were obtained from Charles River Laboratories (Wilmington, Mass.) and were maintained according to NIH standards. All mice were group housed (5 mice/cage) under clean-room conditions in sterile micro-isolator cages on a 12-hour light/dark cycle in a room maintained at 21-22° C. and 40-50% humidity. Mice were fed irradiated standard rodent diet and water ad libitum. All animals were housed in a Laboratory Animal Medicine facility that is fully accredited by the American Association for Assessment and Accreditation of Laboratory Animal Care (AAALAC). All procedures involving animals were conducted in compliance with the NIH Guide for the Care and Use of Laboratory Animals and all protocols were approved by an Internal Animal Care and Use Committee (IACUC).

The human leukemic MV4-11 cell line was obtained from the American Type Culture Collection (ATCC Number: CRL-9591) and propagated in RPMI medium containing 10% FBS (fetal bovine serum) and 5 ng/mL GM-CSF (R&D Systems). MV4-11 cells are derived from a patient with childhood acute myelomonocytic leukemia with an 11q23 translocation resulting in a MLL gene rearrangement and containing an FLT3-ITD mutation (AML subtype M4)(1,2). MV4-11 cells express constitutively active phosphorylated FLT3 receptor as a result of a naturally occurring FLT3/ITD mutation. Strong anti-tumor activity against MV4-11 tumor growth in the nude mouse tumor xenograft model is anticipated to be a desirable quality of the invention.

In pilot growth studies, the following conditions were identified as permitting MV4-11 cell growth in nude mice as subcutaneous solid tumor xenografts: Immediately prior to injection, cells were washed in PBS and counted, suspended 1:1 in a mixture of PBS:Matrigel (BD Biosciences) and then loaded into pre-chilled 1 cc syringes equipped with 25 gauge needles. Female athymic nude mice weighing no less than 20-21 grams were inoculated subcutaneously in the left inguinal region of the thigh with 5×10⁶ tumor cells in a delivery volume of 0.2 mL. For regression studies, the tumors were allowed to grow to a pre-determined size prior to initiation of dosing. Approximately 3 weeks after tumor cell inoculation, mice bearing subcutaneous tumors ranging in size from 106 to 439 mm³ (60 mice in this range) were randomly assigned to treatment groups such that all treatment groups had similar starting mean tumor volumes of ˜200 mm³. Mice were dosed orally by gavage with vehicle (control group) or compound at various doses twice-daily (b.i.d.) during the week and once-daily (q.d.) on weekends. Dosing was continued for 11 consecutive days, depending on the kinetics of tumor growth and size of tumors in vehicle-treated control mice. If tumors in the control mice reached ˜10% of body weight (˜2.0 grams), the study was to be terminated. FLT3 inhibitor compounds were prepared fresh daily as a clear solution (@ 1, 3 and 10 mg/mL) in 20% HPβCD/2% NMP/10 mM Na Phosphate, pH 3-4 (NMP=Pharmasolve, ISP Technologies, Inc.) or other suitable vehicle and administered orally as described above. During the study, tumor growth was measured three times-a-week (M, W, F) using electronic Vernier calipers. Tumor volume (mm³) was calculated using the formula (L×W)²/2, where L=length (mm) and W=width (shortest distance in mm) of the tumor. Body weight was measured three times-a-week and a loss of body weight >10% was used as an indication of lack of compound tolerability. Unacceptable toxicity was defined as body weight loss >20% during the study. Mice were closely examined daily at each dose for overt clinical signs of adverse, drug-related side effects.

On the day of study termination, a final tumor volume and final body weight were obtained on each animal. Mice were euthanized using 100% CO₂ and tumors were immediately excised intact and weighed, with final tumor wet weight (grams) serving as a primary efficacy endpoint.

The time course of the inhibitory effects of FLT3 inhibitor compounds on the growth of MV4-11 tumors is illustrated in FIG. 1. Values represent the mean (±sem) of 15 mice per treatment group. Percent inhibition (% I) of tumor growth was calculated versus tumor growth in the vehicle-treated Control group on the last study day.

Statistical significance versus Control was determined by Analysis of Variance (ANOVA) followed by Dunnett's t-test: * p<0.05; ** p<0.01.

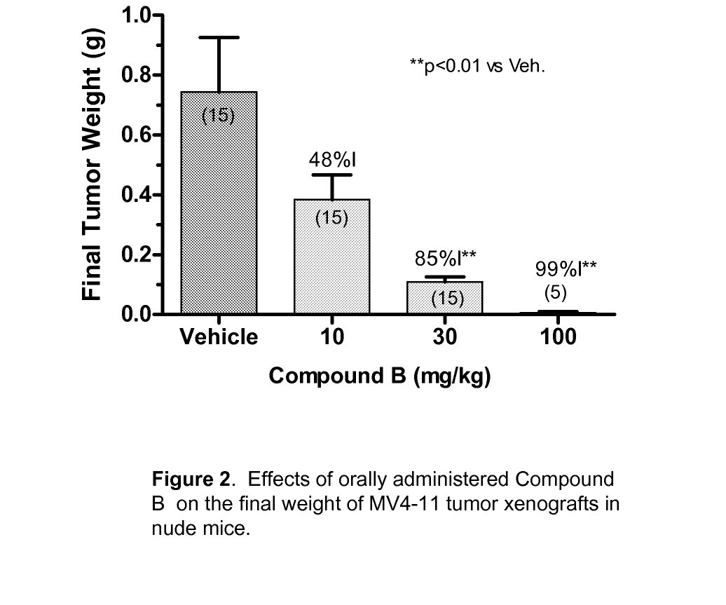

A similar reduction of final tumor weight was noted at study termination. (See FIG. 2). Values represent the mean (±sem) of 15 mice per treatment group, except for the high dose group where only 5 of 15 mice were sacrificed on the day of study termination. Percent Inhibition was calculated versus the mean tumor weight in the vehicle-treated control group. Statistical significance versus Control was determined by ANOVA followed by Dunnett's t-test: ** p<0.01.

FIG. 1: FLT3 inhibitor Compound B administered orally by gavage at doses of 10, 30 and 100 mg/kg b.i.d. for 11 consecutive days, produced statistically significant, dose-dependent inhibition of growth of MV4-11 tumors grown subcutaneously in nude mice. On the last day of treatment (Day 11), mean tumor volume was dose-dependently decreased by 44%, 84% (p<0.01) and 94% (p<0.01) at doses of 10, 30 and 100 mg/kg, respectively, compared to the mean tumor volume of the vehicle-treated group. Tumor regression was observed at doses of 30 mg/kg and 100 mg/kg, with statistically significant decreases of 42% and 77%, respectively, versus the starting mean tumor volumes on Day 1. At the lowest dose tested of 10 mg/kg, modest growth delay was observed (44% I vs Control), however this effect did not achieve statistical significance.

FIG. 2: Following eleven consecutive days of oral dosing, FLT3 inhibitor Compound B produced statistically significant, dose-dependent reductions of final tumor weight compared to the mean tumor weight of the vehicle-treated group, with 48%, 85% (p<0.01) and 99% (p<0.01) decreases at 10, 30 and 100 mg/kg doses, respectively. In some mice, at the high dose of FLT3 inhibitor Compound B, final tumors had regressed to non-palpable, non-detectable tumors.

Mice were weighed three times each week (M, W, F) during the study and were examined daily at the time of dosing for overt clinical signs of any adverse, drug-related side effects. No overt toxicity was noted for FLT3 inhibitor Compound B and no significant adverse effects on body weight were observed during the 11-day treatment period at doses up to 200 mg/kg/day. Overall, across all dose groups for FLT3 inhibitor Compound B the mean loss of body weight was <3% of initial body weight, indicating that the FLT3 inhibitor compounds were well-tolerated.

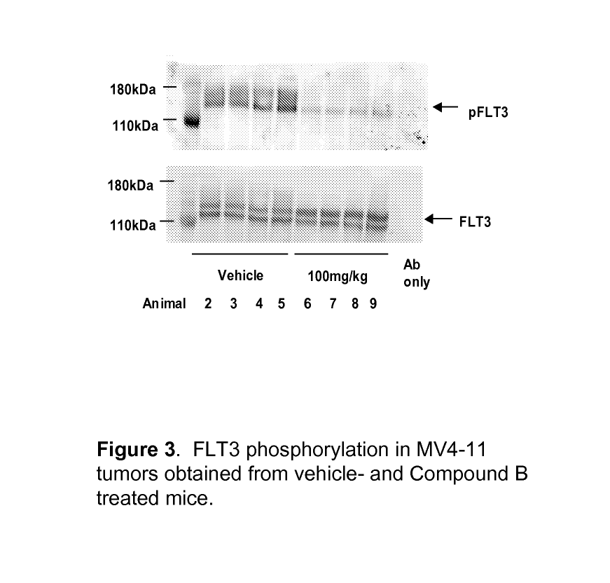

To establish further that FLT3 inhibitor compounds reached the expected target in tumor tissue, the level of FLT3 phosphorylation in tumor tissue obtained from vehicle- and compound-treated mice was measured. Results for FLT3 inhibitor Compound B is shown in FIG. 3. For this pharmacodynamic study, a sub-set of 10 mice from the vehicle-treated control group were randomized into two groups of 5 mice each and then treated with another dose of vehicle or compound (100 mg/kg, po). Tumors were harvested 2 hours later and snap frozen for assessment of FLT3 phosphorylation by immunobloting.

Harvested tumors were processed for immunoblot analysis of FLT3 phosphorylation in the following manner: 100 mg of tumor tissue was dounce homogenized in lysis buffer (50 mM Hepes, 150 mM NaCl, 10% Glycerol, 1% Triton-X-100, 10 mM NaF, 1 mM EDTA, 1.5 mM MgCl₂, 10 mM NaPyrophosphate) supplemented with phosphatase (Sigma Cat# P2850) and protease inhibitors (Sigma Cat #P8340). Insoluble debris was removed by centrifugation at 1000×g for 5 minutes at 4° C. Cleared lysates (15 mg of total potein at 10 mg/ml in lysis buffer) were incubated with 10 μg of agarose conjugated anti-FLT3 antibody, clone C-20 (Santa Cruz cat # sc-479ac), for 2 hours at 4° C. with gentle agitation. Immunoprecipitated FLT3 from tumor lysates were then washed four times with lysis buffer and separated by SDS-PAGE. The SDS-PAGE gel was transfered to nitrocellulose and immunoblotted with anti-phosphotyrosine antibody (clone-4G10, UBI cat. #05-777), followed by alkaline phosphatase-conjugated goat anti-mouse secondary antibody (Novagen cat. # 401212). Detection of protein was done by measuring the fluorescent product of the alkaline phosphatase reaction with the substrate 9H-(1,3-dichloro-9,9-dimethylacridin-2-one-7-yl)phosphate, diammonium salt (DDAO phosphate) (Molecular Probes cat. # D 6487) using a Molecular Dynamics Typhoon Imaging system (Molecular Dynamics, Sunyvale, Calif.). Blots were then stripped and reprobed with anti-FLT3 antibody for normalization of phosphorylation signals.

As illustrated in FIG. 3, a single dose of FLT3 inhibitor Compound B at 100 mg/kg produced a biologically significant reduction in the level of FLT3 phosphorylation in MV4-11 tumors compared to tumors from vehicle-treated mice. (Total FLT3 is shown in the bottom plot.) These results further demonstrate that the comounds of the present invention are in fact interacting with the expected FLT3 target in the tumor.

MV-4-11 tumor-bearing nude mice were prepared as described above, in the aforementioned in vivo evaluation of the oral anti-tumor efficacy of FLT3 inhibitor Compound B.

Anti-Tumor Effect of FLT3 Inhibitor Compound B Administered with Tipifarnib

MV-4-11 tumor-bearing nude mice were prepared as described above, in the aforementioned in vivo evaluation of the oral anti-tumor efficacy of FLT3 inhibitor Compound B alone.

Nude mice with MV-4-11 tumors were randomized to five treatment groups of 15 mice each with mean tumor size was equivalent in each treatment group. Tumor volume (mm3) was calculated using the formula (L×W)2/2, where L=length (mm) and W=width (shortest distance in mm) of the tumor. The starting mean tumor volume for each treatment group was approximately 250 mm3.

Mice were dosed orally twice-daily (bid) during the week and once-daily (qd) on weekends with either Vehicle (20% HPβCD/2% NMP/10 mM Na Phosphate, pH 3-4 (NMP=Pharmasolve, ISP Technologies, Inc.), a sub-efficacious dose of FLT3 inhibitor Compound B (10 mg/kg), an-efficacious dose of FLT3 inhibitor Compound B (20 mg/kg) and Tipifarnib (50 mg/kg) alone or in combination with each dose of FLT3 inhibitor Compound B. Dosing was continued for nine consecutive days. Tumor growth was measured three times during the study using electronic Vernier calipers. Body weight was measured three times during the study and a loss of body weight>10% was used as an indication of lack of compound tolerability.

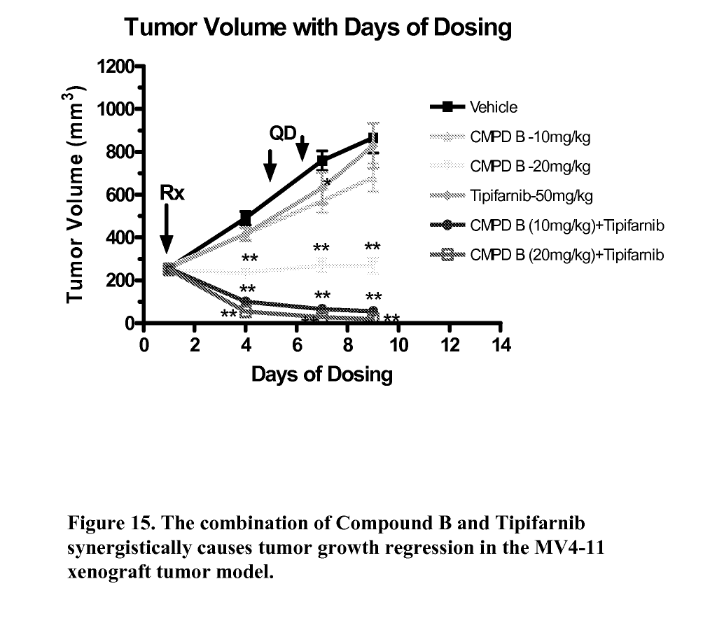

The time course of the effect of treatment with FLT3 inhibitor Compound B and Tipifarnib alone and in combination on the growth of MV-4-11 tumors is illustrated in FIG. 15. As shown, FLT3 inhibitor Compound B administered at a dose of 10 mg/kg bid produced marginal significant inhibition of tumor growth compared to the Vehicle-treated group that reached tumors volumes of approximately 800 mm³. FLT3 inhibitor ‘Compound B administered at a dose of 20 mg/kg bid provided significant inhibition of tumor growth compared to the Vehicle-treated group and completely controlled tumor growth compared to the control. This dose was observed to produce tumor growth stasis but induced no tumor regression (defined as a tumor size less than the tumor size at study initiation). As illustrated in FIG. 15, on the final day of treatment (Day 9), tumor volume was not significantly reduced by Tipifarnib (50 mg/kg) alone when compared to control. Values represent the mean (±sem) of 15 mice per treatment group. Percent inhibition of tumor growth was calculated versus tumor growth in the Vehicle-treated Control group on the last study day. Statistical significance versus Control was determined by ANOVA followed by Dunnett's t-test: * p<0.01.

Again as shown in FIG. 15, Tipifarnib administered as a single agent at a dose of 50 mg/kg was ineffective. However, when both agents were administered orally in combination, there was a statistically significant regression of tumor volume from the mean starting tumor volume on Day 1 when FLT3 inhibitor Compound B was administered at either 10 or 20 mg/kg. On day 9, the mean tumor volume of the group was inhibited by 95% compared to the Vehicle-treated control group. Thus, combination treatment produced an inhibitory effect (ie. tumor regression) that was much greater than either agent administered alone. In point of fact, Tipifarnib (50 mg/kg) and FLT3 inhibitor Compound B alone at 10 mg/kg were essentially inactive while the combination, remarkably provided essentially complete tumor regression.

FIG. 15 illustrates the effects on tumor volume of orally administered FLT3 inhibitor Compound Compound B and Tipifarnib alone or in combination on the growth of MV-4-11 tumor xenografts in nude mice.

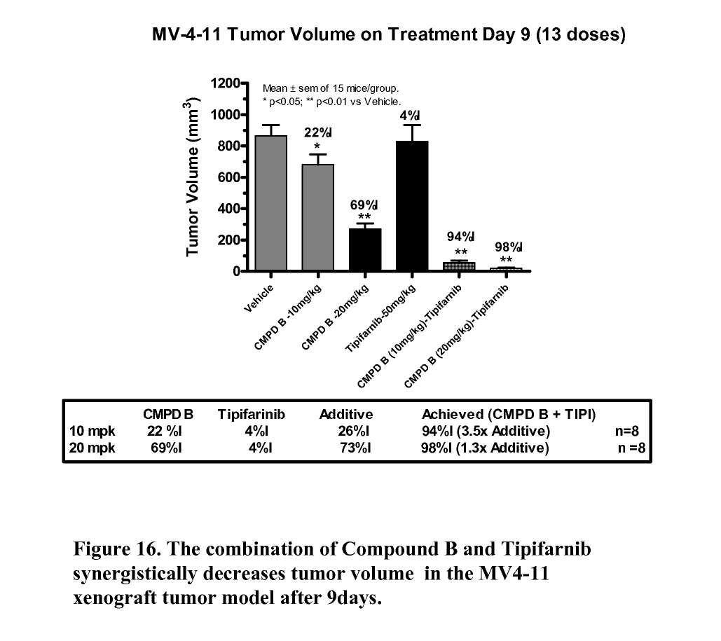

FIG. 16 illustrates the effects of orally administered FLT3 inhibitor Compound B and Tipifarnib alone or in combination on the final volume of MV-4-11 tumor xenografts in nude mice on the final study day. As shown in FIG. 16, at study termination, synergy was noted with combination treatment when the final tumor volumes of each treatment group were compared with the exception that the final tumor weight reached statistical significance.

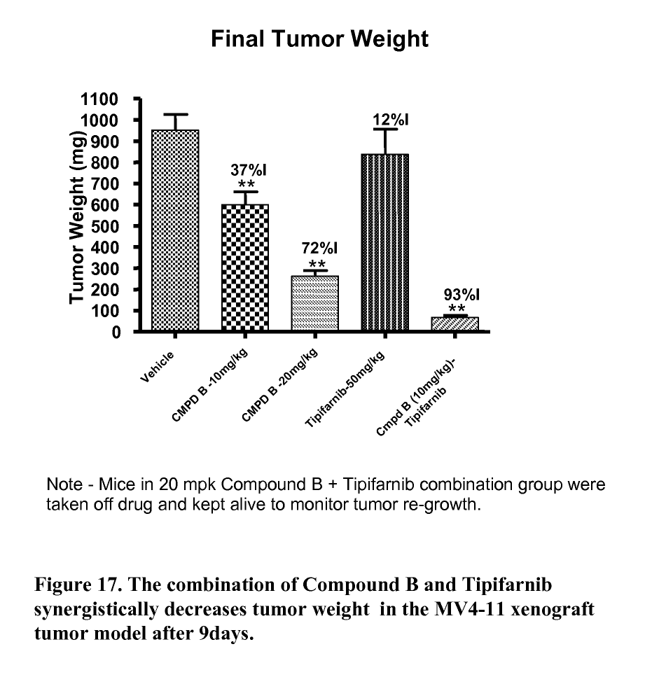

FIG. 17 illustrates the effects of orally administered FLT3 inhibitor Compound B and Tipifarnib alone or in combination on the final tumor weight of MV-4-11 tumor xenografts in nude mice on the terminal study day. As shown in FIG. 17, at study termination, synergy was confirmed by tumor weight measurement in the 10 mg/kg FLT3 inhibitor Compound B/50 mg/kg Tipifarnib combination treatment group when compared to the final tumor weight of the appropriate treatment group when the agents were administered alone.

No overt toxicity was noted and no significant adverse effects on body weight were observed during the 9-day treatment period with either agent alone or in combination. In summary, combination treatment with FLT3 inhibitor Compound B and Tipifarnib produced significantly greater inhibition of tumor growth compared to either FLT3 inhibitor Compound B or Tipifarnib administered alone.

Anti-Tumor Effect of FLT3 Inhibitor Compound D Alone

The oral anti-tumor efficacy of FLT3 inhibitor Compound D of the present invention was evaluated in vivo using a nude mouse MV4-11 human tumor xenograft regression model in athymic nude mice using the method as described above, in the aforementioned in vivo evaluation of the oral anti-tumor efficacy of FLT3 inhibitor Compound B.

MV-4-11 tumor-bearing nude mice were prepared as described above, in the aforementioned in vivo evaluation of the oral anti-tumor efficacy of FLT3 inhibitor Compound B alone.

Female athymic nude mice weighing no less than 20-21 grams were inoculated subcutaneously in the left inguinal region of the thigh with 5×10⁶ tumor cells in a delivery volume of 0.2 mL. For regression studies, the tumors were allowed to grow to a pre-determined size prior to initiation of dosing. Approximately 3 weeks after tumor cell inoculation, mice bearing subcutaneous tumors ranging in size from 100 to 586 mm³ (60 mice in this range; mean of 288±133 mm³ (SD) were randomly assigned to treatment groups such that all treatment groups had statistically similar starting mean tumor volumes (mm³). Mice were dosed orally by gavage with vehicle (control group) or compound at various doses twice-daily (b.i.d.) during the week and once-daily (qd) on weekends. Dosing was continued for 11 consecutive days, depending on the kinetics of tumor growth and size of tumors in vehicle-treated control mice. If tumors in the control mice reached ˜10% of body weight (˜2.0 grams), the study was to be terminated. FLT3 inhibitor Compound D was prepared fresh daily as a clear solution (@ 1, 5 and 10 mg/mL) in 20% HPβCD/D5W, pH 3-4 or other suitable vehicle and administered orally as described above. During the study, tumor growth was measured three times-a-week (M, W, F) using electronic Vernier calipers. Tumor volume (mm³) was calculated using the formula (L×W)²/2, where L=length (mm) and W=width (shortest distance in mm) of the tumor. Body weight was measured three times-a-week and a loss of body weight >10% was used as an indication of lack of compound tolerability. Unacceptable toxicity was defined as body weight loss >20% during the study. Mice were closely examined daily at each dose for overt clinical signs of adverse, drug-related side effects.

On the day of study termination (Day 12), a final tumor volume and final body weight were obtained on each animal. Mice were euthanized using 100% CO₂ and tumors were immediately excised intact and weighed, with final tumor wet weight (grams) serving as a primary efficacy endpoint.

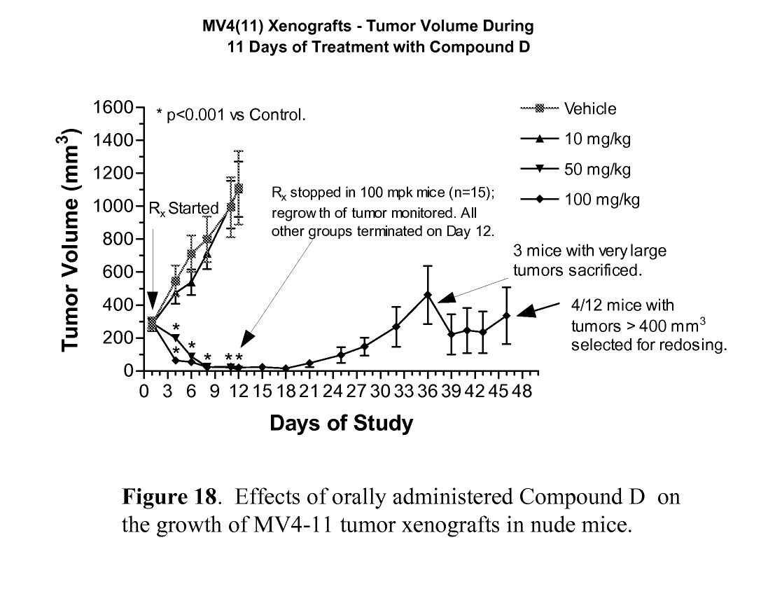

The time course of the inhibitory effects of FLT3 inhibitor Compound D of the present invention on the growth of MV4-11 tumors is illustrated in FIG. 18. Values represent the mean (±sem) of 15 mice per treatment group. Percent inhibition (% I) of tumor growth was calculated versus tumor growth in the vehicle-treated Control group on the last study day. Statistical significance versus Control was determined by Analysis of Variance (ANOVA) followed by Dunnett's t-test: * p<0.05; ** p<0.01.

As seen in FIG. 18, FLT3 inhibitor Compound D of the present invention, administered orally by gavage at doses of 10, 50 and 100 mg/kg b.i.d. for 11 consecutive days, produced statistically significant, dose-dependent inhibition of growth of MV4-11 tumors grown subcutaneously in nude mice. On the last day of treatment (Day 11), mean tumor volume was dose-dependently decreased with nearly 100% inhibition (p<0.001) at doses of 50 and 100 mg/kg, compared to the mean tumor volume of the vehicle-treated group. FLT3 inhibitor Compound D of the present invention produced tumor regression at doses of 50 mg/kg and 100 mg/kg, with statistically significant decreases of 98% and 93%, respectively, versus the starting mean tumor volumes on Day 1. At the lowest dose tested of 10 mg/kg, no significant growth delay was observed compared to the vehicle-treated control group. When dosing was stopped on Day 12 in the 100 mg/kg treated dose group and the tumor was allowed to re-grow, only 6/12 mice showed papable, measureable tumor on study day 34.

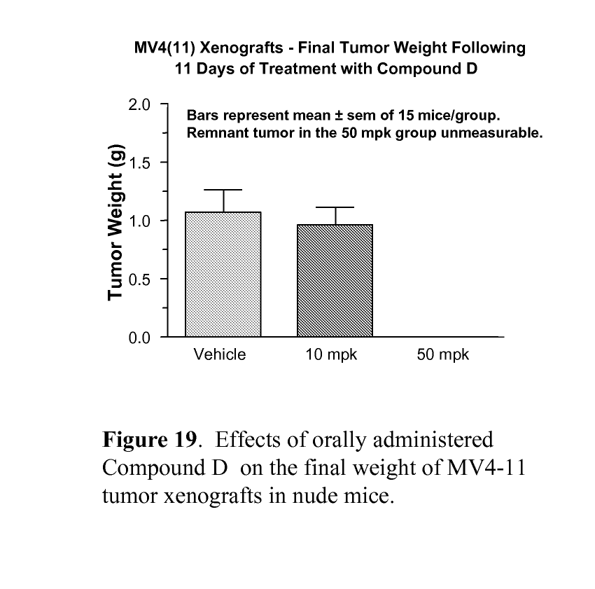

FLT3 inhibitor Compound D of the present invention produced virtually complete regression of tumor mass as indicated by no measurable remant tumor at study termination. (See FIG. 19). Bars on the graph of FIG. 19 represent the mean sem) of 15 mice per treatment group. As shown, there was no significant decrease in final tumor weight at the 10 mg/kg dose, consistent with the tumor volume data in FIG. 18. At the dose of 50 mg/kg, there is no bar represented on the graph since there was no measurable tumor mass detectable in these mice at termination, consistent with the complete regression of tumor volume noted in FIG. 18. The 100 mg/kg dose group is not represented on this graph since these mice were taken off drug and remnant tumor was allowed to regrow as stated above.

Following eleven consecutive days of oral dosing, FLT3 inhibitor Compound D of the present invention produced dose-dependent reductions of final tumor weight compared to the mean tumor weight of the vehicle-treated group, with complete regression of tumor mass noted at the 50 mg/kg dose. (See FIG. 19).

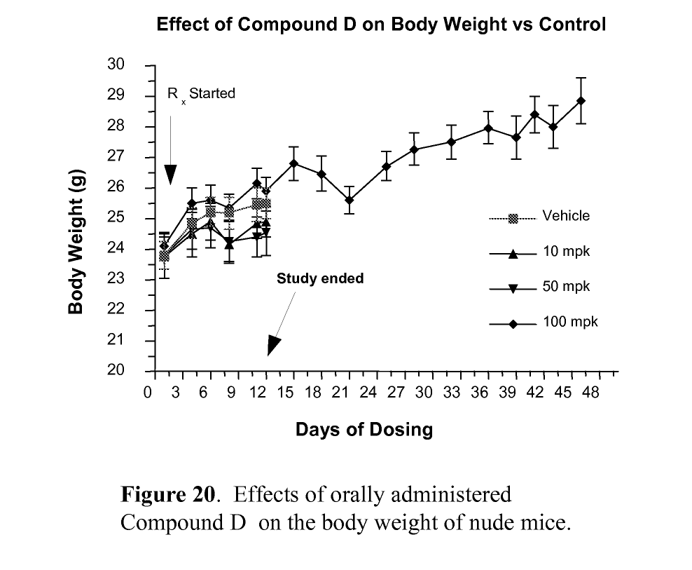

Mice were weighed three times each week (M, W, F) during the study and were examined daily at the time of dosing for overt clinical signs of any adverse, drug-related side effects. No overt toxicity was noted for FLT3 inhibitor Compound D of the present invention and no significant adverse effects on body weight were observed during the 11-day treatment period at doses up to 200 mg/kg/day (See FIG. 20). Overall, across all dose groups, there was no significant loss of body weight compared to the starting body weight, indicating that FLT3 inhibitor Compound D of the present invention was well-tolerated.

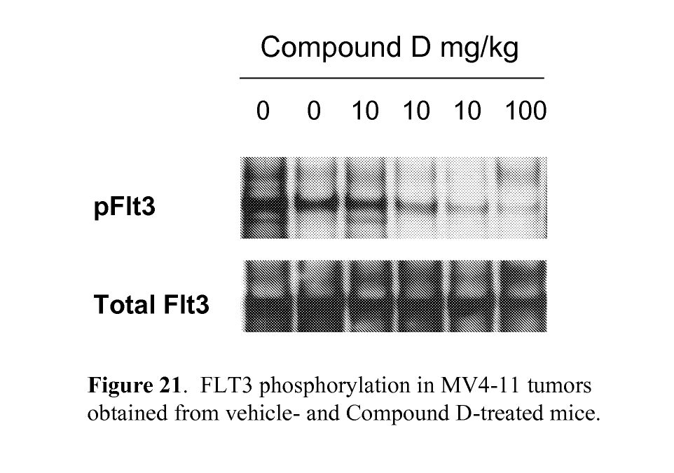

To establish further that FLT3 inhibitor Compound D of the present invention reached the expected target in tumor tissue, the level of FLT3 phosphorylation in tumor tissue obtained from vehicle- and compound-treated mice was measured. Results for FLT3 inhibitor Compound D of the present invention are shown in FIG. 21. For this pharmacodynamic study, a sub-set of 6 mice from the vehicle-treated control group were randomized into three groups of 2 mice each and then treated with another dose of vehicle or compound (10 and 100 mg/kg, po). Tumors were harvested 6 hours later and snap frozen for assessment of FLT3 phosphorylation by western blots.

Harvested tumors were frozen and processed for immunoblot analysis of FLT3 phosphorylation in the following manner: 200 mg of tumor tissue was dounce homogenized in lysis buffer (50 mM Hepes, 150 mM NaCl, 10% Glycerol, 1% Triton-X-100, 10 mM NaF, 1 mM EDTA, 1.5 mM MgCl₂, 10 mM NaPyrophosphate) supplemented with phosphatase (Sigma Cat# P2850) and protease inhibitors (Sigma Cat #P8340). Insoluble debris was removed by centrifugation at 1000×g for 5 minutes at 4° C. Cleared lysates (15 mg of total potein at 10 mg/ml in lysis buffer) were incubated with 10 μg of agarose conjugated anti-FLT3 antibody, clone C-20 (Santa Cruz cat # sc-479ac), for 2 hours at 4° C. with gentle agitation.

Immunoprecipitated FLT3 from tumor lysates were then washed four times with lysis buffer and separated by SDS-PAGE. The SDS-PAGE gel was transfered to nitrocellulose and immunoblotted with anti-phosphotyrosine antibody (clone-4G10, UBI cat. #05-777), followed by alkaline phosphatase-conjugated goat anti-mouse secondary antibody (Novagen cat. # 401212). Detection of protein was done by measuring the fluorescent product of the alkaline phosphatase reaction with the substrate 9H-(1,3-dichloro-9,9-dimethylacridin-2-one-7-yl)phosphate, diammonium salt (DDAO phosphate) (Molecular Probes cat. # D 6487) using a Molecular Dynamics Typhoon Imaging system (Molecular Dynamics, Sunyvale, Calif.). Blots were then stripped and reprobed with anti-FLT3 antibody for normalization of phosphorylation signals.

As illustrated in FIG. 21, a single dose of FLT3 inhibitor Compound D of the present invention at 100 mg/kg produced a biologically significant reduction in the level of FLT3 phosphorylation (top panel, tumor 5 and 6) in MV4-11 tumors compared to tumors from vehicle-treated mice (tumor 1 and 2). (Total FLT3 is shown in the bottom plot.) There was also a partial reduction of phosphorylation in animals treated with 10 mg/kg of the compound (tumor 3-4). These results further demonstrate that the compound of the present invention is in fact interacting with the expected FLT3 target in the tumor.

Anti-Tumor Effect of FLT3 Inhibitor Compound D Administered with Tipifarnib

To demonstrate in vivo synergy of the combination of FLT3 inhibitor Compound D and Tipifarnib in MV-4-11 xenograft model, tumor-bearing nude mice were prepared as described above, in the aforementioned in vivo evaluation of the oral anti-tumor efficacy of FLT3 inhibitor Compound B alone.

Nude mice with MV-4-11 tumors were randomized to four treatment groups of 10 mice each with mean tumor size was equivalent in each treatment group. Tumor volume (mm3) was calculated using the formula (L×W)2/2, where L=length (mm) and W=width (shortest distance in mm) of the tumor. The starting mean tumor volume for each treatment group was approximately 250 mm3.

Mice were dosed orally twice-daily (bid) during the week and once-daily (qd) on weekends with either Vehicle (20% HPβ-CD, pH 3-4) or sub-efficacious doses of FLT3 inhibitor Compound D (25 mg/kg) or Tipifarnib (50 mg/kg) alone or in combination. Dosing was continued for sixteen consecutive days. Tumor growth was measured three times-a-week (Monday, Wednesday, Friday) using electronic Vernier calipers. Body weight was measured three times-a-week and a loss of body weight >10% was used as an indication of lack of compound tolerability.

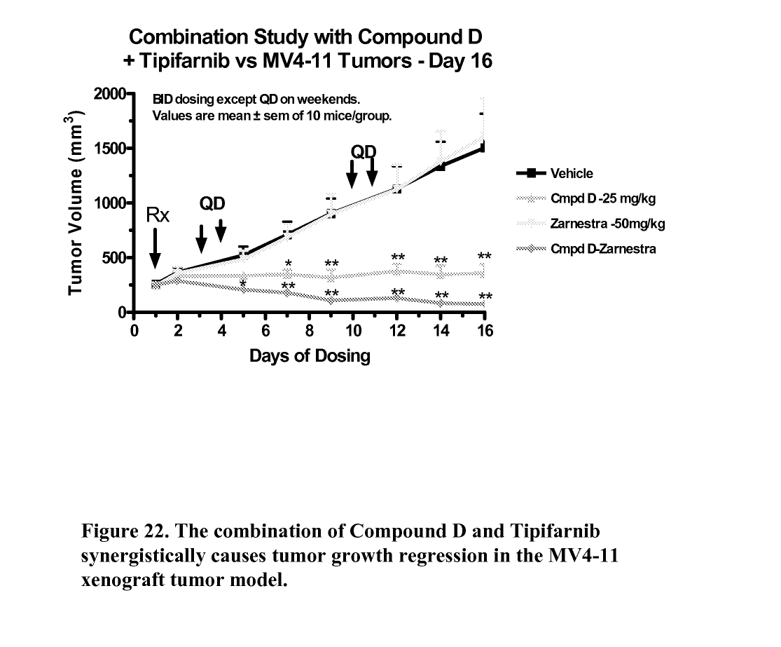

The time course of the effect of treatment with FLT3 inhibitor Compound D and Tipifarnib alone and in combination on the growth of MV-4-11 tumors is illustrated in FIG. 22. As shown, FLT3 inhibitor Compound D administered at a dose of 25 mg/kg bid produced stasis of tumor growth compared to the Vehicle-treated group which reached tumors volumes of approximately 1500 mm³. As illustrated in FIG. 22, on the final day of treatment (Day 16), tumor volume was significantly inhibited by 76% compared to the vehicle-treated control group. Values represent the mean (±sem) of 10 mice per treatment group. Percent inhibition of tumor growth was calculated versus tumor growth in the Vehicle-treated Control group on the last study day. Statistical significance versus Control was determined by ANOVA followed by Dunnett's t-test: * p<0.01.

As shown in FIG. 22, Tipifarnib administered as a single agent at a dose of 50 mg/kg was ineffective. However, when both agents were administered orally in combination, there was a statistically significant regression of tumor volume from the mean starting tumor volume on Day 1. On day 16, the mean tumor volume of the group was inhibited by 95% compared to the Vehicle-treated control group. Thus, combination treatment produced an inhibitory effect (ie. tumor regression) that was approximately 1.3 times the additive effect of each agent given alone, indicating synergy (see FIG. 22).

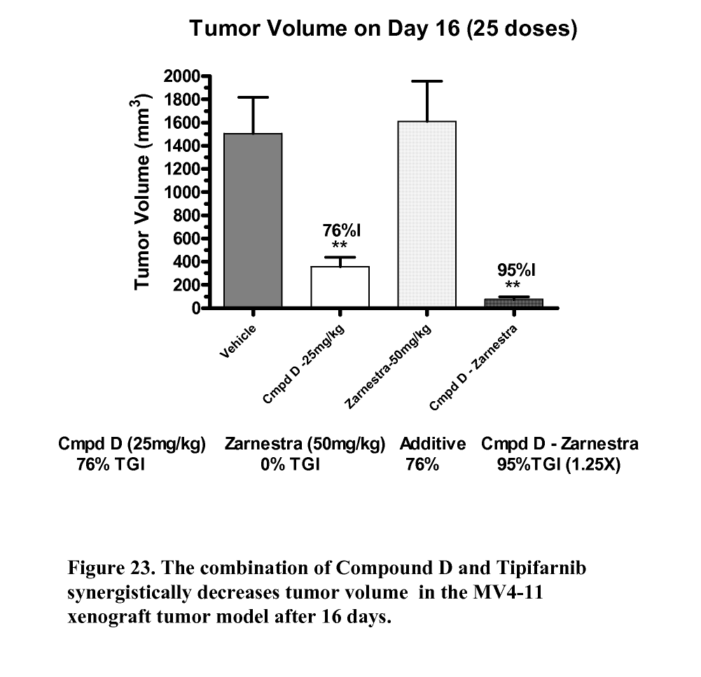

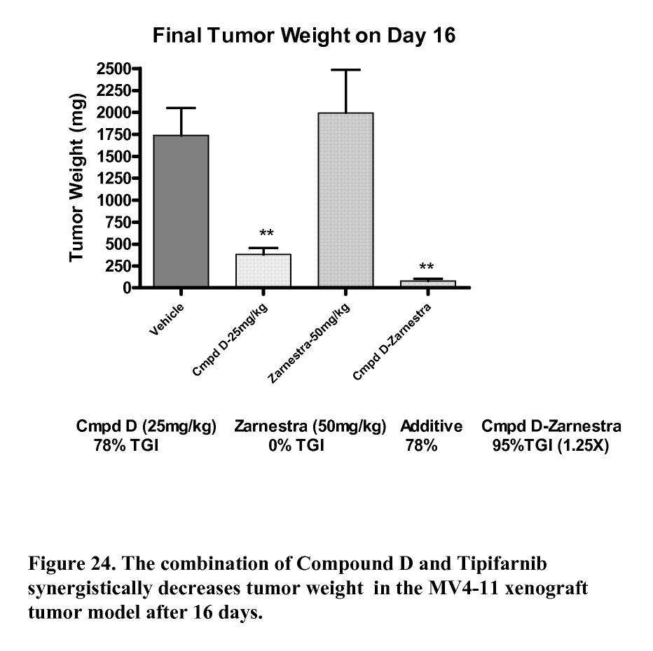

FIG. 23 illustrates the effects on tumor volume of orally administered FLT3 inhibitor Compound D and Tipifarnib alone or in combination on the growth of MV-4-11 tumor xenografts in nude mice. FIG. 24 illustrates the effects of orally administered FLT3 inhibitor Compound D and Tipifarnib alone or in combination on the final weight of MV-4-11 tumor xenografts in nude mice. As shown in FIG. 24, at study termination, similar synergy was noted with combination treatment when the final tumor weights of each treatment group were compared.

No overt toxicity was noted and no significant adverse effects on body weight were observed during the 16-day treatment period with either agent alone or in combination. Plasma and tumor samples were collected two hours after the last dose of compounds for determination of drug levels. In summary, combination treatment with FLT3 inhibitor Compound D and Tipifarnib produced significantly greater inhibition of tumor growth compared to either FLT3 inhibitor Compound D or Tipifarnib administered alone.

### CONCLUSIONS

Herein we provide significant evidence that the combination of an FTI and a FLT3 inhibitor synergistically inhibits the growth of and induces the death of FLT3-dependent cells in vitro and in vivo (such as AML cells derived from patients with FLT3-ITD mutations). In vitro studies, in multiple FLT3-dependent cell lines, demonstrated synergistic inhibition of AML cell proliferation with the FTI/FLT3 inhibitor combination by both the combination index method of Chou and Talalay and the median effect method using a combination of single sub-optimal doses of each compound. Additionally, the combination of an FTI and a FLT3 inhibitor induced dramatic cell death in FLT3-dependent AML cells. This effect on apoptotsis induction was significantly greater than either agent alone. This synergistic effect of an FTI/FLT3 inhibitor combination was observed for multiple, structurally distinct FLT3 inhibitors and two different FTIs. Accordingly, this synergistic inhibition of proliferation and induction of apoptosis would occur for any FLT3 inhibitor/FTI combination. Interestingly, the combination of the FTI Tipifarnib with a FLT3 inhibitor significantly increases the potency of FLT3 inhibitor mediated decrease in FLT3 receptor signaling. Furthermore, the synergy observed using in vitro methods was recapitulated in an in vivo tumor model using FLT3-dependent AML cells (MV4-11) with the combination of the FTI Tipifarnib and two chemically distinct FLT3 inhibitors (FLT3 inhibitor Compounds B and D). Accordingly, this effect would be seen for any FLT3 inhibitor/FTI combination. To our knowledge, this is the first time that synergistic AML cell killing has been observed with the combination of an FTI and a FLT3 inhibitor. Additionally, the synergies observed in the combination were not obvious to those skilled in the art based on previous data. The observed synergy is likely related to FTIs known inhibition small GTPase (Ras and Rho) and NfkB driven proliferation and survival and the FLT3 inhibitors' ability to decrease proliferation and survival signaling by the FLT3 receptor. Additionally, the FTI/FLT3 inhibitor combination had significant effects on the activity of the FLT3 receptor itself. Although the mechanism for this is currently unknown, it is likely to have a significant role in both the inhibition of cell proliferation and activation of cell death observed with the FLT3 inhibitor/FTI combination. In sum, these studies represent a novel treatment paradigm for FLT3 disorders, particularly hematological malignancies expressing wild-type or mutant FLT3 and the basis for the design of clinical trials to test FTI and FLT3 inhibitor combinations for the treatment of FLT3 disorders, particularly AML, ALL and MDS.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of reducing or inhibiting FLT3 tyrosine kinase expression or activity in a subject comprising the administration of a FLT3 kinase inhibitor and a farnesyl transferase inhibitor to the subject, wherein the FLT3 kinase inhibitor comprises a compound selected from the group consisting of Formula I′ and Formula II′: 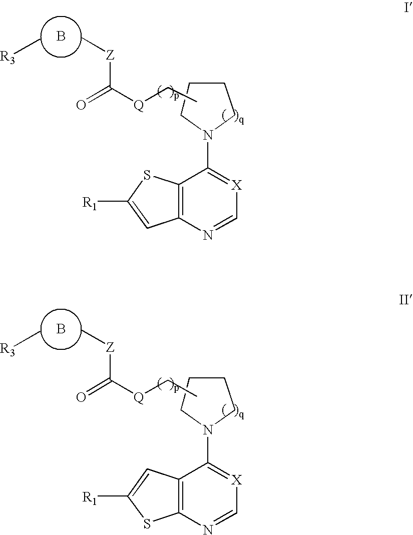
and N-oxides, pharmaceutically acceptable salts, and stereochemical isomers thereof, wherein: 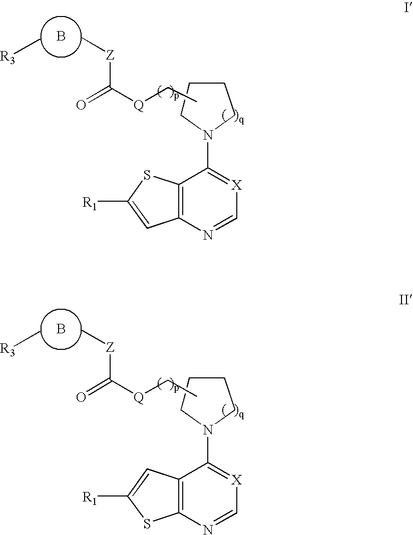
q is 0, 1 or 2;
p is 0 or 1;
Q is NH, N(alkyl), O, or a direct bond;
X is N or CH;
Z is NH, N(alkyl), or CH₂;
B is aryl, cycloalkyl, heteroaryl, or a nine to ten membered benzo-fused heteroaryl;
R₁ is: 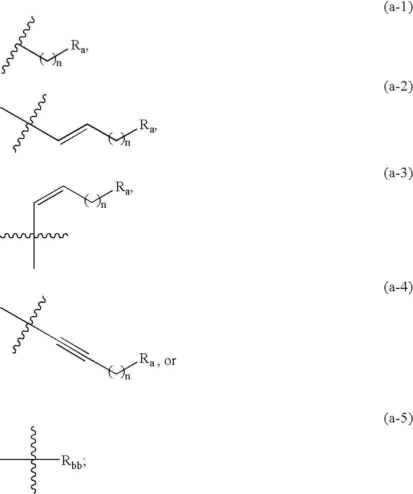
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

2. A method of treating disorders related to FLT3 tyrosine kinase expression or activity in a subject comprising the administration of a FLT3 kinase inhibitor and a farnesyl transferase inhibitor to the subject, wherein the FLT3 kinase inhibitor comprises a compound selected from the group consisting of Formula I′ and Formula II′: 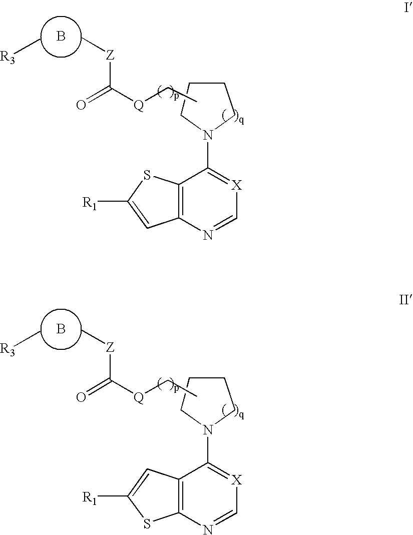
q is 0, 1 or 2;
p is 0 or 1;
Q is NH, N(alkyl), O, or a direct bond;
X is N or CH;
Z is NH, N(alkyl), or CH₂;
B is aryl, cycloalkyl, heteroaryl, or a nine to ten membered benzo-fused heteroaryl;
R₁ is: 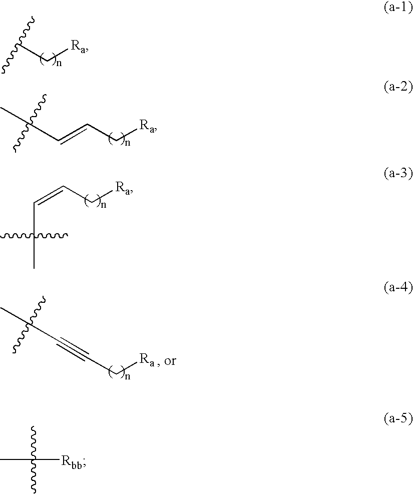
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

3. (canceled)

4. The method of claim 2 further comprising administering to the subject a prophylactically effective amount of chemotherapy.

5. The method of claim 2 further comprising administering to the subject a prophylactically effective amount of radiation therapy.

6. The method of claim 2 further comprising administering to the subject a prophylactically effective amount of gene therapy.

7. The method of claim 2 further comprising administering to the subject a prophylactically effective amount of immunotherapy.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method of treating in a subject a disorder related to FLT3, comprising administering to the subject a therapeutically effective amount of (1) a first pharmaceutical composition comprising a FLT3 kinase inhibitor and a pharmaceutically acceptable carrier, and (2) a second pharmaceutical composition comprising a farnesyl transferase inhibitor and a pharmaceutically acceptable carrier, wherein the FLT3 kinase inhibitor comprises a compound selected from the group consisting of Formula I′ and Formula II′: 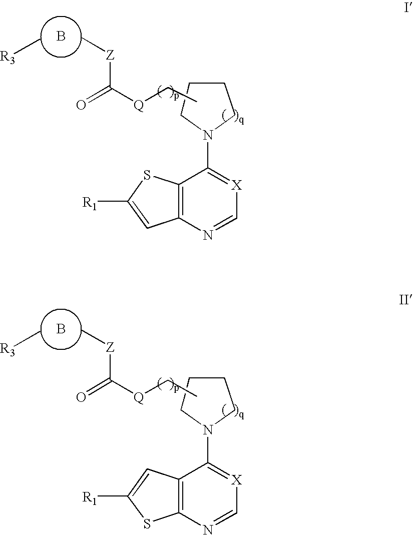
q is 0, 1 or 2;
p is 0 or 1;
Q is NH, N(alkyl), O, or a direct bond;
X is N or CH;
Z is NH, N(alkyl), or CH₂;
B is aryl, cycloalkyl, heteroaryl, or a nine to ten membered benzo-fused heteroaryl;
R₁ is: 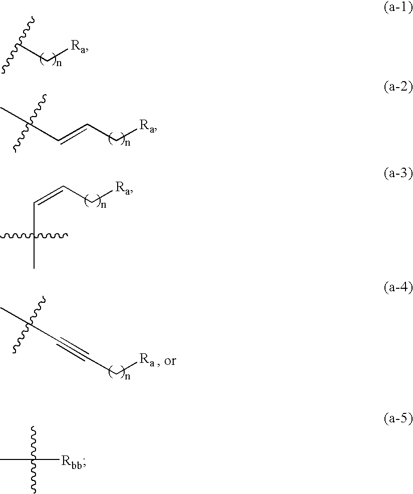
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

34. The method of claim 33 further comprising administering to the subject a therapeutically effective amount of chemotherapy.

35. The method of claim 33 further comprising administering to the subject a therapeutically effective amount of radiation therapy.

36. The method of claim 33 further comprising administering to the subject a therapeutically effective amount of gene therapy.

37. The method of claim 33 further comprising administering to the subject a therapeutically effective amount of immunotherapy.

38. A method of treating in a subject a disorder related to FLT3, comprising administering to the subject a therapeutically effective amount of a pharmaceutical composition comprising a FLT3 kinase inhibitor, a farnesyl transferase inhibitor and a pharmaceutically acceptable carrier, wherein the FLT3 kinase inhibitor comprises a compound selected from the group consisting of Formula I′ and Formula II′: 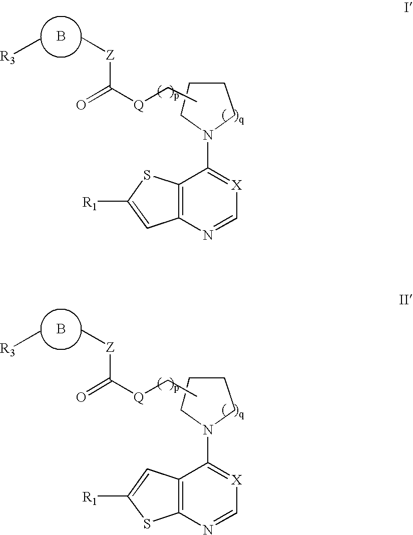
q is 0, 1 or 2;
p is 0 or 1;
Q is NH, N(alkyl), O, or a direct bond;
X is N or CH;
Z is NH, N(alkyl), or CH₂;
B is aryl, cycloalkyl, heteroaryl, or a nine to ten membered benzo-fused heteroaryl;
R₁ is: 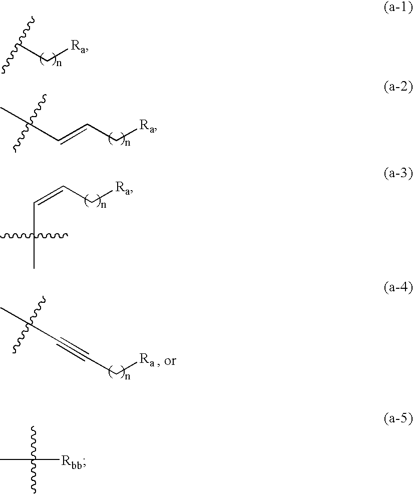
wherein
n is 1, 2, 3 or 4;
Rₐ is hydrogen, heteroaryl optionally substituted with R₅, hydroxyl, alkylamino, dialkylamino, oxazolidinonyl optionally substituted with R₅, pyrrolidinonyl optionally substituted with R₅, piperidinonyl optionally substituted with R₅, cyclic heterodionyl optionally substituted with R₅, heterocyclyl optionally substituted with R₅, —COOR_{y}, —CONR_{w}Rₓ, —N(R_{y})CON(R_{w})(Rₓ), —N(R_{w})C(O)ORₓ, —N(R_{w})COR_{y}, —SR_{y}, —SOR_{y}, —SO₂R_{y}, —NR_{w}SO₂R_{y}, —NR_{w}SO₂Rₓ, —SO₃R_{y}, or —OSO₂NR_{w}Rₓ;
R_{bb} is hydrogen, halogen, aryl, heteroaryl, or heterocyclyl;
R₅ is one, two, or three substituents independently selected from: halogen, cyano, trifluoromethyl, amino, hydroxyl, alkoxy, —C(O)alkyl, —SO₂alkyl, —C(O)N(alkyl)₂, alkyl, —C₍₁₋₄₎alkyl-OH, or alkylamino;
R_{w} and Rₓ are independently selected from: hydrogen, alkyl, alkenyl, aralkyl, or heteroaralkyl, or R_{w} and Rₓ may optionally be taken together to form a 5 to 7 membered ring, optionally containing a heteromoiety selected from O, NH, N(alkyl), SO₂, SO, or S;
R_{y} is selected from: hydrogen, alkyl, alkenyl, cycloalkyl, aryl, aralkyl, heteroaralkyl, or heteroaryl; and
R₃ is one or more substituents, optionally present, and independently selected from: alkyl, alkoxy, halogen, alkoxyether, hydroxyl, thio, nitro, cycloalkyl optionally substituted with R₄, heteroaryl optionally substituted with R₄, alkylamino, heterocyclyl optionally substituted with R₄, partially unsaturated heterocyclyl optionally substituted with R₄, —O(cycloalkyl), pyrrolidinone optionally substituted with R₄, phenoxy optionally substituted with R₄, —CN, —OCHF₂, —OCF₃, —CF₃, halogenated alkyl, heteroaryloxy optionally substituted with R₄, dialkylamino, —NHSO₂alkyl, thioalkyl, or —SO₂alkyl; wherein R₄ is independently selected from: halogen, cyano, trifluoromethyl, amino, hydroxyl, alkoxy, —C(O)alkyl, —CO₂alkyl, —SO₂alkyl, —C(O)N(alkyl)₂, alkyl, or alkylamino.

39. The method of claim 38 further comprising administering to the subject a therapeutically effective amount of chemotherapy.

40. The method of claim 38 further comprising administering to the subject a therapeutically effective amount of radiation therapy.

41. The method of claim 38 further comprising administering to the subject a therapeutically effective amount of gene therapy.

42. The method of claim 38 further comprising administering to the subject a therapeutically effective amount of immunotherapy.

43. The method of claim 38 further comprising administering to the subject a therapeutically effective amount of chemotherapy.

44. A method as defined claim 33, wherein the farnesyl transferase inhibitor comprises a compound of formula (I): 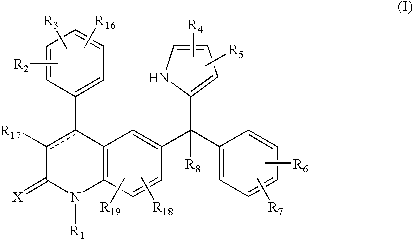
a stereoisomeric form thereof, a pharmaceutically acceptable acid or base addition salt thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, wherein Alk¹ is C₁₋₆alkanediyl, R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);
R⁴ and R⁵ each independently are hydrogen, halo, Ar¹, C₁₋₆alkyl, hydroxy-C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula
—O—CH₂—O— (c-1), or
—CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula
—O—R¹⁰ (b-1),
—S—R¹⁰ (b-2),
—N—R¹¹R¹² (b-3),
wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, a radical or formula -Alk²-OR¹³ or -Alk²-NR¹⁴R¹⁵;
R¹¹ is hydrogen, C₁₋₁₂alkyl, Ar¹ or Ar²C₁₋₆alkyl;
R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆ alkylcarbonyl, amino, C₁₋₆alkylamino, C₁₋₆alkylcarbonylamino, or a radical of formula -Alk²-OR¹³ or -Alk²-NR¹⁴R¹⁵; wherein Alk² is C₁₋₆alkanediyl; R¹³ is hydrogen, C₁₋₆alkyl, C₁₋₆alkylcarbonyl, hydroxyC₁₋₆alkyl, Ar¹ or Ar²C₁₋₆alkyl; R¹⁴ is hydrogen, C₁₋₆alkyl, Ar¹ or Ar²C₁₋₆alkyl; R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

45. The method of claim 44 wherein said farnesyl transferase inhibitor comprises a compound of formula (I) wherein X is oxygen and the dotted line represents a bond.

46. The method of claim 44 wherein said farnesyl transferase inhibitor comprises a compound of formula (I) wherein R¹ is hydrogen, C₁₋₆alkyl, C₁₋₆alkyloxyC₁₋₆alkyl or, mono- or di(C₁₋₆alkyl)aminoC₁₋₆alkyl; R² is halo, C₁₋₆alkyl, C₂₋₆alkenyl, C₁₋₆alkyloxy, trihalomethoxy, or hydroxyC₁₋₆alkyloxy; and R³ is hydrogen.

47. The method of claim 44 wherein said farnesyl transferase inhibitor comprises a compound of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₂alkyl and R¹² is hydrogen, C₁₋₆alkyl, C₁₋₆alkyloxy, C₁₋₆alkyloxyC₁₋₆alkylcarbonyl, hydroxy, or a radical of formula -Alk²-OR¹³ wherein R¹³ is hydrogen or C₁₋₆alkyl.

48. The method of claim 44 wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

49. The method as defined in claim 33, wherein said FLT3 kinase inhibitor comprises a compound of Formula I′ and Formula II′ wherein R_{w} and Rₓ are independently selected from hydrogen, alkyl, alkenyl, aralkyl, or heteroaralkyl, or may optionally be taken together to form a 5 to 7 membered ring, selected from the group consisting of:

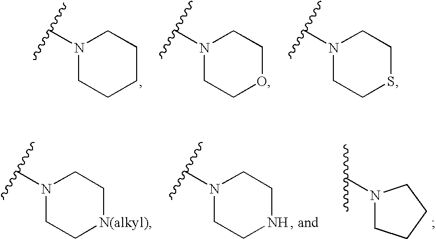

50. The method as defined in claim 33, wherein said FLT3 kinase inhibitor comprises a compound of Formula I′ and Formula II′ wherein q is 1 or 2; X is N; and B is aryl or heteroaryl.

51. The method as defined in claim 33, wherein said FLT3 kinase inhibitor comprises a compound of Formula I′ and Formula II′ wherein
Q is NH, O, or a direct bond;
Z is NH or CH₂; and
R₃ is one or more substituents, optionally present, and independently selected from: alkyl, alkoxy, halogen, alkoxyether, cycloalkyl optionally substituted with R₄, alkylamino, heterocyclyl optionally substituted with R₄, —O(cycloalkyl), phenoxy optionally substituted with R₄, dialkylamino, or —SO₂alkyl.

52. The method as defined in claim 33, wherein said FLT3 kinase inhibitor comprises a compound of Formula I′ and Formula II′ wherein
R₁ is: 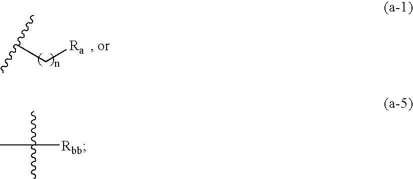
Rₐ is hydrogen, hydroxyl, alkylamino, dialkylamino, heterocyclyl optionally substituted with R₅, —CONR_{w}Rₓ, —N(R_{y})CON(R_{w})(Rₓ), —N(R_{w})C(O)ORₓ, —N(R_{w})COR_{y}, —SO₂R_{y}, —NR_{w}SO₂R_{y}, or —NR_{w}SO₂Rₓ; and
R₃ is one substituent selected from: alkyl, alkoxy, halogen, alkoxyether, cycloalkyl optionally substituted with R₄, alkylamino, heterocyclyl optionally substituted with R₄, —O(cycloalkyl), phenoxy optionally substituted with R₄, dialkylamino, or —SO₂alkyl.

53. The method as defined in claim 33, wherein said FLT3 kinase inhibitor comprises a compound of Formula I′ and Formula II′ wherein
q is 1 or 2;
p is 0 or 1;
Q is NH, O, or a direct bond;
Z is NH or CH₂;
B is phenyl or pyridyl;
X is N;
R₁ is: 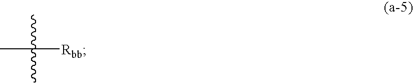
wherein
R_{bb} is hydrogen, halogen, aryl, or heteroaryl; and
R₃ is one substituent selected from: alkyl, alkoxy, heterocyclyl, —O(cycloalkyl), phenoxy, or dialkylamino.

54. The method as defined in claim 33, wherein said FLT3 kinase inhibitor comprises a compound of Formula I′ and Formula II′ wherein
p is 0;
Q is NH or O;
Z is NH;
R_{bb} is hydrogen; and
R₃ is one substituent selected from: alkyl, —O(cycloalkyl), phenoxy, or dialkylamino.

55. The method as defined in claim 33, wherein said FLT3 kinase inhibitor comprises a compound of Formula I′ and Formula II′ selected from the group consisting of:

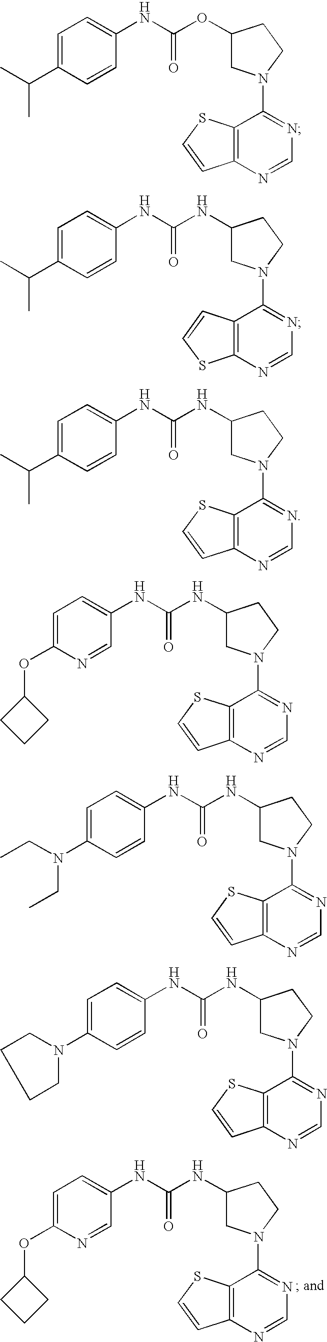

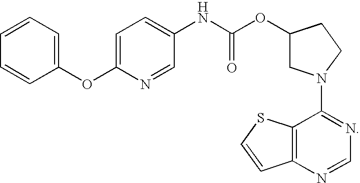

56. The method as defined in claim 33, wherein said FLT3 kinase inhibitor comprises a compound of Formula I′ and Formula II′ selected from the group consisting of:

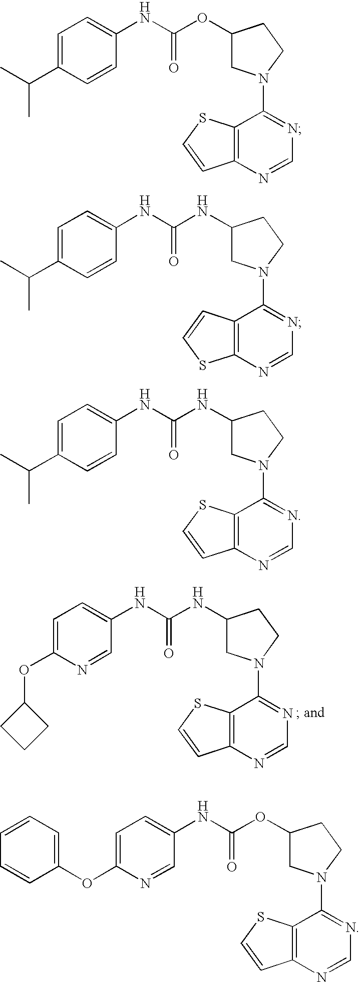

57. (canceled)

58. The method of claim 49, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

59. The method of claim 50, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

60. The method of claim 51, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

61. The method of claim 52, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

62. The method of claim 53, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

63. The method of claim 54, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

64. The method of claim 55, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

65. The method of claim 56, wherein the farnesyl transferase inhibitor is (+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

66. (canceled)
